(19) United States
(12) Patent Application Publication
(10) Pub. No.: US 2025/0213572 A1
(43) Pub. Date: Jul. 3, 2025

(54) TYK2 INHIBITORS AND USES THEREOF

(71) Applicant: Takeda Pharmaceutical Company Limited, Osaka (JP)

(72) Inventors: Alan John COLLIS, Lexington, MA (US); Vinayak HOSAGRAHARA, Arlington, MA (US); Bhaskar SRIVASTAVA, Newton, MA (US)

(21) Appl. No.: 18/850,466

(22) PCT Filed: Mar. 24, 2023
(86) PCT No.: PCT/US2023/064902

§ 371 Date: Sep. 24, 2024

Related U.S. Application Data
(60) Provisional application No. 63/490,664, filed on Mar. 16, 2023.
(60) Provisional application No. 63/371,033, filed on Aug. 10, 2022.
(60) Provisional application No. 63/269,943, filed on Mar. 25, 2022.

(51) Int. Cl. A61K 31/519; A61P 17/06; A61P 19/02

(52) U.S. Cl. CPC A61K 31/519; A61P 17/06; A61P 19/02

(57) ABSTRACT

Compounds, compositions thereof, and methods of using the same in dosing protocols for the inhibition of TYK2, and the treatment of inflammatory disorders including psoriasis and psoriatic arthritis are described.

### CLAIM OF PRIORITY

This application claims priority to U.S. Provisional Patent Application No. 63/269,943 filed Mar. 25, 2022, U.S. Provisional Patent Application No. 63/371,033 filed Aug. 10, 2022, and U.S. Provisional Patent Application No. 63/490,664 filed Mar. 16, 2023, each of which is incorporated by reference in its entirety.

### TECHNICAL FIELD

The present invention relates to methods of administering a non-receptor tyrosine-protein kinase 2 (TYK2) inhibitor, such as N-((1R,2R)-2-methoxycyclobutyl)-7-(methylamino)-5-((2-oxo-2H-[1,2′-bipyridin]-3-yl)amino)pyrazolo[1,5-a]pyrimidine-3-carboxamide (Compound 1), and uses thereof for treating inflammatory disorders (e.g., psoriasis and psoriatic arthritis).

### BACKGROUND

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxins, and H₂O₂), cytokines (e.g., interleukin-1 (IL-1), interleukin-8 (IL-8), interleukin-12 (IL-12), interleukin-23 (IL-23), and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, some cancers, cardiovascular diseases, allergies and asthma, Alzheimer's disease, and hormone-related diseases.

TYK2 catalyzes the phosphorylation of STAT proteins downstream of a number of cytokine receptors, including the Type I interferon receptor and the IL-12 and IL-23 receptors. The activation of TYK2-dependent receptors by their cytokine ligands results in the activation of STAT-dependent transcription and cellular functional responses specific for the receptors and cell types on which they are expressed. The cytokine signaling pathways regulated by TYK2 play key roles in several immune-mediated disorders. The cytokine IL-12 is essential for the development of Type 1 T-helper cells (Th1) which produce interferon-gamma, a major effector molecule in systemic autoimmune disorders such as systemic lupus erythematosus. The cytokine IL-23 is central for the expansion and survival of Th17 cells and innate lymphoid cells, both of which have been shown to play key pathogenic roles in autoimmunity. IL-23 stimulation drives the production of key proinflammatory cytokines by Th17 cells, including IL-17A, IL-17F, and IL-22, all of which are effector molecules important for pathogenesis of conditions such as psoriasis, psoriatic arthritis, and ankylosing spondylitis. Inhibition of TYK2 would be expected to impact multiple immune-mediated disorders through its effects on the IL-23/Th17/Th22 axis, IL-12-mediated Th1 functions, and Type I interferon-driven modulation of diverse immune pathways and cell types.

Accordingly, there remains a need to find TYK2 inhibitors useful as therapeutic agents. The present disclosure satisfies this need and provides other related advantages.

### SUMMARY

It has been found that certain TYK2 inhibitors are suitable for oral administration to a patient for treating an inflammatory disorder (e.g., psoriasis, psoriatic arthritis, or an inflammatory bowel disease (IBD) such as ulcerative colitis or Crohn's disease). Accordingly, in one aspect, a method of treating psoriasis, psoriatic arthritis, ulcerative colitis, Crohn's disease, or inflammatory bowel disease to a patient in need thereof, can include administering to a patient a therapeutically effective amount of a TYK2 inhibitor (e.g., Compound 1), or a pharmaceutically acceptable salt thereof, or a pharmaceutical composition thereof. Compound 1 has the following structure:

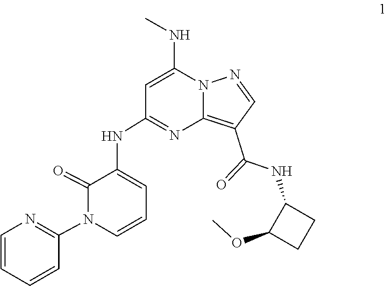 [SMILES: CNc1cc(Nc2cccn(-c3ccccn3)c2=O)nc2c(C(=O)N[C@@H]3CC[C@H]3OC)cnn12]

Compound 1 has the IUPAC name N-((1R,2R)-2-methoxycyclobutyl)-7-(methylamino)-5-((2-oxo-2H-[1,2′-bipyridin]-3-yl)amino)pyrazolo[1,5-a]pyrimidine-3-carboxamide.

In some embodiments, a mean decrease in psoriasis area severity index (PASI) of up to about 50% (e.g., about 30%, about 47%, or about 48%) can be achieved; or a mean decrease in psoriasis area severity index (PASI) of about 25% to about 50% ecan be achieved. A mean decrease in psoriasis area severity index (PASI) of about 50% to 75%, or of about 75% to 100%, can be achieved.

In some embodiments, a Physician Global Assessment (PGA) of 0 or 1 can be achieved; or a Physician Global Assessment (PGA) of 0 can be achieved.

In some embodiments, a Dermatology Life Quality Index (DLQI) score can be reduced by an amount between about 1 to about 3, about 3 to about 6, about 6 to about 9, about 9 to about 12, about 12 to about 15, about 15 to about 18, about 18 to about 21, about 21 to about 24, about 24 to about 27, or about 27 to about 30.

In some embodiments, a Dermatology Life Quality Index (DLQI) score can be reduced by an amount greater than about 5, or by an amount between about 6 to about 9.

In some embodiments, the patient's Body Surface Area (BSA) can be reduced by a number of percentage points between about 1 to about 10, about 10 to about 20, about 20 to about 30, about 30 to about 40, about 40 to about 50, about 50 to about 60, about 60 to about 70, about 70 to about 80, about 80 to about 90, or about 90 to about 100.

In some embodiments, a reduction in pain numeric rating scale (NRS) of about 1, 2, 3, 4, 5, 6, 7, 8, 9, or 10 can be achieved.

In some embodiments, a reduction in pruritus numeric rating scale (NRS) of about 1, 2, 3, 4, 5, 6, 7, 8, 9, or 10 can be achieved.

In some embodiments, an inhibition of IL-12/18-induced IFNγ production between about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, can be achieved.

In some embodiments, the patient can have plaque psoriasis; the plaque psoriasis can be moderate-to-severe-plaque psoriasis.

In some embodiments, the patient can have psoriasis and psoriatic arthritis. The patient can have psoriatic arthritis.

In some embodiments, the method can achieve at least an American College of Rheumatology (ACR) 20 response.

In some embodiments, the method can achieve an improvement in tender joint count, swollen joint count, Patient Global Assessment of Psoriatic Arthritis, Patient Global Assessment of Psoriatic Arthritis Pain, Physician Global Assessment of Psoriatic Arthritis, Health Assessment Questionnaire-Disability Index (HAQ-DI) score, dactylitis count, Leed's Enthesitis Index, Minimal Disease Activity, Disease Activity Index for Psoriatic Arthritis (DAPSA), or a Psoriasis Area Severity Index (PASI)-75 at Week 12 among subjects with ≥3% body surface area (BSA) psoriatic involvement at Day 1, or a static Physician Global Assessment of 0 or 1 and at least a 2-point improvement from baseline at Week 12.

In some embodiments, the method can achieve an improvement in circulating cytokines and/or an inflammatory biomarker, a 36-Item Short Form Health Survey, Functional Assessment of Chronic Illness—Fatigue, Disease Activity Score 28 with high-sensitivity C-Reactive Protein (hsCRP), Psoriatic Disease Activity Score at week 12 of treatment, Psoriatic Arthritis Response Criteria at week 12 of treatment, a mean decrease in psoriasis area severity index (PASI) of 25% or greater, or an improvement in BSA with psoriatic involvement at week 12, or an improvement in Physician Global Assessment of Psoriasis at week 12 of treatment.

In another aspect, a method of inhibiting of interferon gamma (IFNγ) production, can include administering to a patient in need thereof a therapeutically effective amount of Compound 1, or a pharmaceutically acceptable salt thereof, or a composition thereof. In some embodiments, the method is associated with improvements in PASI, such as those described above and elsewhere herein.

In some instances, the disclosed methods and uses of administering Compound 1 achieving certain pharmacokinetic parameters of the disclosure has certain advantages in treating psoriasis and psoriatic arthritis. For example, in some embodiments, a Tₘₐₓ of Compound 1 in plasma is achieved in about 3 hours to about 6 hours or a t_{1/2} of Compound 1 in plasma is achieved in about 17 hours to about 37 hours.

In some embodiments, Compound 1 or a pharmaceutically acceptable salt thereof is administered at a dose of up to about 200 mg (e.g., from about 20 mg to about 100 mg) to the patient. In other embodiments, Compound 1 or a pharmaceutically acceptable salt thereof is administered at a dose of about 2 mg, about 5 mg, about 10 mg, about 15 mg, or about 30 mg to the patient.

In some embodiments, Compound 1 or a pharmaceutically acceptable salt thereof is administered to the patient as a single daily dose or in multiple daily doses. In other embodiments, Compound 1 or a pharmaceutically acceptable salt thereof is administered to the patient daily for between 2 weeks and 4 weeks. In some embodiments, Compound 1 or a pharmaceutically acceptable salt thereof is administered for at least 2 weeks or at least 4 weeks, for example until the patient's psoriasis or psoriatic arthritis improves. In some embodiments, Compound 1 or a pharmaceutically acceptable salt thereof is administered chronically to treat the patient. In some embodiments, Compound 1 or a pharmaceutically acceptable salt thereof is administered orally to the patient (e.g., a human).

In some embodiments, the patient can have a PASI of at least about 12 prior to the administration.

In some embodiments, the patient can have a PGA of at least about 3 prior to the administration.

In some embodiments, the patient can have a BSA of at least about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, prior to the administration.

In some embodiments, the psoriasis does not comprise erythrodermic, pustular, predominantly guttate psoriasis, or drug-induced psoriasis.

In some embodiments, a serum Cₘₐₓ between about 25 ng/ml to about 50 ng/ml, about 50 ng/ml to about 100 ng/ml, about 100 ng/ml to about 125 ng/ml, about 125 ng/ml to about 150 ng/ml, about 150 ng/ml to about 175 ng/ml, about 175 ng/ml to about 200 ng/ml, about 200 ng/ml to about 225 ng/ml, about 225 ng/ml to about 250 ng/ml, about 250 ng/ml to about 275 ng/ml, or about 275 ng/ml to about 300 ng/ml, can be achieved.

In another aspect, a method of treating moderate-to-severe plaque psoriasis in a patient in need thereof, which includes administering a therapeutically effective amount of Compound 1 at a daily dose of from about 5 mg to about 30 mg is provided.

In another aspect, the use of Compound 1, or a pharmaceutically acceptable salt thereof in the treatment of psoriasis or psoriatic arthritis is provided.

These and other aspects of this disclosure will be apparent upon reference to the following detailed description.

### BRIEF DESCRIPTION OF THE FIGURES

FIG. 1 is a schematic depicting a study diagram for Example 4.

FIG. 2 is a schematic depicting a study diagram for Example 5.

FIG. 3 shows graphs depicting comparative selectivity of maximum drug concentration (C_{MAX}) to JAK1/3 IC₅₀ based on PK modeling for deucravacitinib and Compound 1. Note: Compound 1 data is PBMC pSTAT IC₅₀ for TYK2; values for JAKs are >50,000 ng/mL.

FIG. 4 shows graphs depicting comparative levels of TYK2 inhibition—modeled based on Phase 1a data, not evaluated in head-to-head study. Note: Compound 1 data for hWB IC₅₀ of 25 ng/mL, Hill slope 1.

FIG. 5 is a graph depicting Compound 1 Phase 1a pharmacodynamic data based on IFN gamma inhibition.

FIG. 6 is an image and data depicting Compound 1 reduced epidermal thickness and resolved expression of the K16 biomarker in patients across the three dosing cohorts at four weeks (17 patients evaluated).

FIG. 7 shows graphs depicting Compound 1 treatment for 28 days reduced the RNA expression levels of key cytokines and psoriasis gene signatures in lesional skin. Quantification of mRNA from skin biopsies showed decreases in the expression of several psoriasis-related genes by RT-PCR (such as KRT16, IL17A, IL17F, and IL22) and up to 50% improvement in the expression of skin transcriptomes previously associated with psoriasis in lesional skin through microarray analysis (p<0.001 compared to placebo treatment).

FIG. 8 are schematics depicting X-ray crystallographic results of Compound 1 bound to TYK2 JH2 domain (top) and molecular docking studies of Compound 1 bound to JAK1 JH2 domain (bottom).

FIG. 9 shows graphs depicting Compound 1 suppressed disease activity in a dose-related manner in a rat adjuvant-induced arthritis (AIA) efficacy model. On day 0, the adjuvant was injected. On day 9, baseline ankle diameters were measured. On days 12-20, daily ankle diameter measurements were taken. On day 20, a final necroscopy and paw weight measure were taken. Compound 1 was administered daily for all 20 days. Compound 1 exposure was assessed on Day 19. IC_{50/90} reference lines (top panel) represent inhibitory concentrations in a rat whole-blood TYK2-dependent IFNα-induced IP10 assay. Concentration-dependent suppression of TYK2-dependent IL12-induced INFγ production also observed in rat (not shown).

FIG. 10 shows graphs depicting the steady state exposure (plasma concentration) of Compound 1 administered to mice administered anti-CD40 (top panel) and the colon weights and colon histologies of the mice (bottom panel).

FIG. 11 shows graphs depicting pharmacokinetics of Compound 1 measured in healthy volunteers (PO; QD) Study 101 and Study 104. Peak mean plasma concentrations at median Tₘₐₓ were 4-6 hrs on day 1 and day 14. Cₘₐₓ and AUC_{0-2 h} increased in an approximately dose-proportional manner from 5-75 mg. Oral Tim was consistent across doses: 16.5-30.7 hrs. The accumulation ratios for Cₘₐₓ and AUC were 2.2-2.9× and 2.5-3.2×, respectively.

FIG. 12 shows a graph depicting steady-state plasma concentrations of Compound 1 based on steady-state exposures obtained from the 20 mg and 35 mg cohorts of Study 101 (Example 1), 5 mg cohort of Study 102 (Example 2) and the 50 mg cohort of Study 104. IC_{50/90} reference lines represent inhibitory concentrations in a human whole-blood TYK2-dependent IFNα-induced IP10 assay. The 35 and 50 mg doses cover the IC₉₀ for 24 h.

FIG. 13 is a graph depicting the fraction of patients achieving PASI 75 at week 12 in the mITT analysis set of the study of Example 4.

FIG. 14 is a graph depicting the fraction of patients achieving PASI 75, PASI 90, and PASI 100 at week 12 in the study of Example 4.

FIG. 15 is a graph depicting the fraction of patients achieving PGA 0/1 at week 12 in the study of Example 4.

FIG. 16A is a graph depicting mean DLQI score for patients in the study of Example 4 at baseline and at week 12. FIG. 16B: change from baseline in DLQI at Week 12.

FIGS. 17A-17E are graphs depicting hematological parameters and CPK at weeks 0, 1, 2, 4, 8, 12 and 16. FIG. 17A, neutrophil count (ANC). FIG. 17B, lymphocyte count (ALC). FIG. 17C, hemoglobin. FIG. 17D, platelets. FIG. 17E, creatine kinase (CPK).

FIGS. 18A-18E are graphs depicting hepatic and renal parameters. FIG. 18A, alanine aminotransferase (ALT). FIG. 18B, aspartate aminotransferase (AST). FIG. 18C, bilirubin (total). FIG. 18D, creatine. FIG. 18E, estimated glomerular filtration rate (eGFR).

FIGS. 19A-19E are graphs depicting lipid parameters. FIG. 19A, cholesterol (total). FIG. 19B, triglycerides. FIG. 19C, HDL cholesterol. FIG. 19D, LDL cholesterol. FIG. 19E, LDL/HDL ratio.

### DETAILED DESCRIPTION OF CERTAIN EMBODIMENTS

TYK2 is an obligate mediator of signaling via IL-23, IL-12 and the type I interferon receptors and is a validated therapeutic target in psoriasis and psoriatic arthritis. Compound 1 is an allosteric TYK2 inhibitor with exquisite selectivity, and has been studied in 3 placebo-controlled Phase I studies: Studies 1 and 2: Healthy volunteers (HV) treated with single doses (5-200 mg, N=5 or 6 in each of 6 groups; placebo, N=6); and multiple doses (20 mg-100 mg daily, N=6 in each of 4 groups; placebo, N=4 in each of two groups) for 2 weeks; Study 3: Patients with moderate to severe psoriasis treated daily for 4 weeks (5, 10 or 30 mg, N=21 total; placebo, N=5).

Compound 1 has also been studied in a randomized, double-blind, placebo-controlled phase 2b trial in moderate-to-severe psoriasis (Example 4: 2 mg QD, N=50; 5 mg QD, N=52; 15 mg QD, N=53, 30 mg QD, N=52; placebo, N=52). Compound 1 has also been studied in a phase 2b, randomized, multi-center, double-blind, placebo-controlled, multiple-dose study in subjects with active psoriatic arthritis (Example 5: 5 mg QD, 15 mg QD, and 30 mg QD).

Compound 1 disclosed herein is an oral, allosteric selective TYK2 inhibitor that is used for the treatment of psoriasis, psoriatic arthritis and other inflammatory and autoimmune diseases. There are currently no TYK2 inhibitors approved, and Compound 1 is the most selective TYK2 inhibitor currently in clinical development. Furthermore, Compound 1's selectivity and potential to provide higher levels of TYK2 inhibition for a longer period with once-daily (QD) dosing may confer clinical and ultimately commercial advantages over other TYK2 inhibitors in development. TYK2 is a member of the Janus kinase (JAK) family of kinases, a class of intracellular signaling proteins that regulate chronic inflammation in inflammatory and autoimmune diseases. Although JAK inhibition can be effective in treating inflammatory and autoimmune diseases, it also can produce on-target safety issues by modulating a broad variety of cytokine pathways. As a result, while JAK inhibitors have become established oral treatments for numerous inflammatory and autoimmune diseases, their clinical utility is constrained by elevated risk of infections and other side effects that have resulted in U.S. Food and Drug Administration (FDA)-mandated boxed warnings and dosing limitations as part of their labeling. Designing selective JAK inhibitors and dosing protocols that directly and specifically inhibit the intended kinase function is challenging due to the structural similarity between the catalytic (orthosteric or JH1) sites for drug targeting on the JAK catalytic domains. Based on human genetic data and growing clinical evidence for the selectivity of allosteric TYK2 inhibitors, the present approach of selective allosteric inhibition of TYK2 provides an optimal balance of achieving strong efficacy while potentially avoiding safety concerns associated with broader JAK inhibition for the treatment of multiple inflammatory and autoimmune diseases.

Across the 3 Phase I studies, the absorption of Compound 1 was rapid (median Tₘₐₓ 3-6 hours), increases in exposure were approximately dose proportional and half-life (t_{1/2}) was 17-37 hours. Pharmacodynamics was tested in a cytokine-induced ex vivo assay using whole blood samples. Treatment with Compound 1 led to rapid inhibition of interferon gamma (IFNγ) production; increasing exposure correlated with increased inhibition of IFNγ. No serious adverse events (SAEs) or deaths were observed. Adverse events (AEs) observed included acneiform dermatitis, papular rash, aphthous ulcer, headache and diarrhea. Laboratory abnormalities that occurred in more than one subject with a Common Terminology Criteria for Adverse Events (CTCAE) grade of 2 or greater included neutropenia, lymphopenia, elevated levels of creatine phosphokinase, and non-fasting triglyceride elevation. Exploratory efficacy in psoriasis patients demonstrated improvement for all doses tested (mean decreases in psoriasis area severity index (PASI) of 30%, 47%, and 48% for 5 mg, 10 mg, and 30 mg respectively; versus 26% for placebo). The TYK2 inhibitors described herein are provided by oral administration at the doses and schedules described herein.

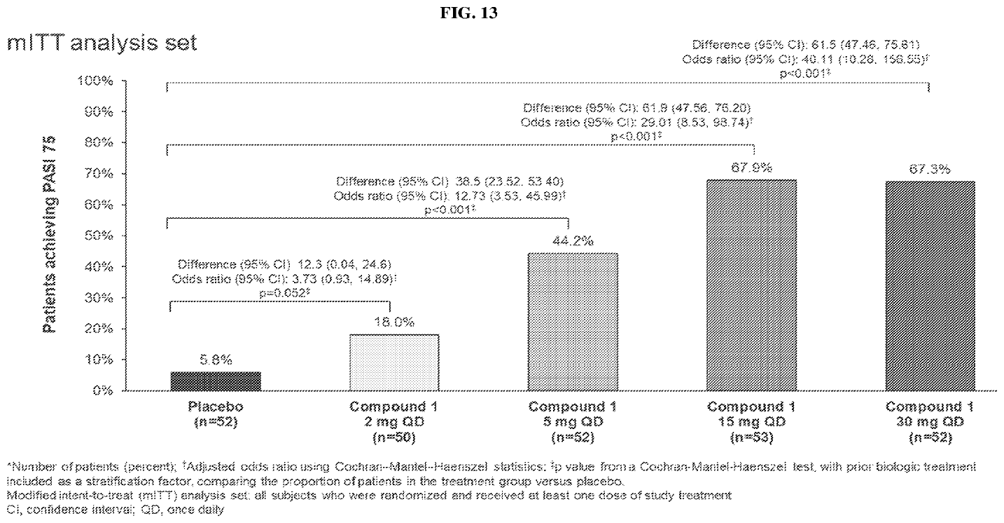

In the Phase 2b study of plaque psoriasis, the fraction of patients achieving PASI 75 were 5.8% for placebo; 18.0% at 2 mg dose; 44.2% for 5 mg dose; 67.9% for 15 mg dose; and 67.3% for 30 mg dose. See FIG. 13; further results and discussion are below in Example 4.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment,” “some embodiments,” or “an embodiment” means that a particular feature, structure, or characteristic described in connection with the embodiment is included in at least one embodiment. Thus, the appearances of the phrases “in one embodiment,” “in some embodiments,” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In one aspect, a method of treating psoriasis, psoriatic arthritis, ulcerative colitis, Crohn's disease, or inflammatory bowel disease (IBD) in a patient in need thereof, can include administering to a patient a therapeutically effective amount of Compound 1:

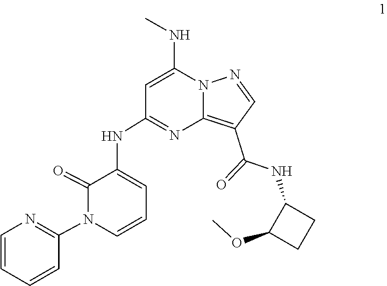 [SMILES: CNc1cc(Nc2cccn(-c3ccccn3)c2=O)nc2c(C(=O)N[C@@H]3CC[C@H]3OC)cnn12]

or a pharmaceutically acceptable salt thereof or a pharmaceutical composition thereof.

In some embodiments, the administration is daily for a period between about 1 day to about 7 days, about 1 week to about 3 weeks, about 3 weeks to about 6 weeks, about 6 weeks to about 9 weeks, about 9 weeks to 12 weeks, about 12 weeks to about 15 weeks, or about 15 weeks to about 18 weeks.

In some embodiments, the method is directed to treatment of psoriasis and/or psoriatic arthritis.

In some embodiments, the method is directed to treatment of plaque psoriasis, e.g., moderate-to-severe plaque psoriasis.

In some embodiments, a mean decrease in psoriasis area severity index (PASI) of 25% or greater is achieved.

In some embodiments, a mean decrease in psoriasis area severity index (PASI) of about 35% or greater is achieved.

In some embodiments, a mean decrease in psoriasis area severity index (PASI) of about 45% or greater is achieved.

In some embodiments, a mean decrease in psoriasis area severity index (PAST) of about 25% to 50% is achieved.

In some embodiments, a mean decrease in psoriasis area severity index (PAST) of about 50% to 75% is achieved.

In some embodiments, a mean decrease in psoriasis area severity index (PAST) of about 75% to 100% is achieved.

In some embodiments, a Physician Global Assessment (PGA) of 0, 1, or 2 is achieved.

In some embodiments, a Physician Global Assessment (PGA) of 0 or 1 is achieved.

In some embodiments, a Physician Global Assessment (PGA) of 0 is achieved.

In some embodiments, a Dermatology Life Quality Index (DLQI) score is reduced by an amount between about 1 to about 3, about 3 to about 6, about 6 to about 9, about 9 to about 12, about 12 to about 15, about 15 to about 18, about 18 to about 21, about 21 to about 24, about 24 to about 27, or about 27 to about 30.

In some embodiments, Dermatology Life Quality Index (DLQI) score can be reduced by an amount greater than about 5,

In some embodiments, Dermatology Life Quality Index (DLQI) score can be reduced by an amount between about 6 to about 9.

In some embodiments, the patient's Body Surface Area (BSA) is reduced by a number of percentage points between about 1 to about 10, about 10 to about 20, about 20 to about 30, about 30 to about 40, about 40 to about 50, about 50 to about 60, about 60 to about 70, about 70 to about 80, about 80 to about 90, or about 90 to about 100.

In some embodiments, a reduction in pain numeric rating scale (NRS) of about 1, 2, 3, 4, 5, 6, 7, 8, 9, or 10 is achieved.

In some embodiments, a reduction in pruritus numeric rating scale (NRS) of about 1, 2, 3, 4, 5, 6, 7, 8, 9, or 10 is achieved.

In some embodiments, an inhibition of IL-12/18-induced IFNγ production between about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, is achieved.

In some embodiments, the patient has both psoriasis and psoriatic arthritis.

In some embodiments, the patient has psoriasis but not psoriatic arthritis.

In some embodiments, the psoriasis is moderate to severe.

In some embodiments, the psoriatic arthritis is moderate to severe.

In some embodiments, the patient has psoriatic arthritis.

In some embodiments, the method achieves at least an American College of Rheumatology (ACR) 20 response.

In some embodiments, the method results in an ACR 20 response after about 12 weeks of treatment. In some embodiments, the method results in an ACR 50 response. In some embodiments, the method results in an ACR 70 response. In some embodiments, the method results in an ACR 50 or ACR 70 response after about 12 weeks of treatment. In some embodiments, the method results in an ACR 20 response after 18 weeks. In some embodiments, the method results in an ACR 50 response after 18 weeks. In some embodiments, the method results in an ACR 70 response after 18 weeks. In some embodiments, the ACR response is measured after 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. ACR20 is a composite measure defined as both improvement of 20% in the number of tender (68) and number of swollen (66) joints, and a 20% improvement in three of the following five criteria: patient global assessment of psoriatic arthritis, physician global assessment of psoriatic arthritis, patient pain scale, disability history questionnaire (i.e., HAQ-DI) and an acute phase reactant (i.e., erythrocyte sedimentation rate [ESR] or hsCRP). In some embodiments, the improvement in acute phase reactant is measured by hsCRP. In some embodiments, such improvements are measured against the patient's baseline score before beginning treatment.

In some embodiments, the method achieves an improvement in tender joint count, swollen joint count, Patient Global Assessment of Psoriatic Arthritis, Patient Global Assessment of Psoriatic Arthritis Pain, Physician Global Assessment of Psoriatic Arthritis, Health Assessment Questionnaire-Disability Index (HAQ-DI) score, dactylitis count, Leed's Enthesitis Index, Minimal Disease Activity, Disease Activity Index for Psoriatic Arthritis (DAPSA), or a Psoriasis Area Severity Index (PASI)-75 at Week 12 among subjects with ≥3% body surface area (BSA) psoriatic involvement at Day 1, or a static Physician Global Assessment of 0 or 1 and at least a 2-point improvement from baseline at Week 12.

In some embodiments, the method achieves an improvement in tender joint count. In some embodiments, the method achieves an improvement in swollen joint count. In some embodiments, the method achieves an improvement in Patient Global Assessment of Psoriatic Arthritis. In some embodiments, the method provides an improvement of about 1, 2, 3, 4, 5, 10, 15, 20, 25, 30, 35, 40, 45, 50, or greater than 50; or about 1-5, 2-8, 2-15, 2-25, 3-9, 5-10, 5-20, 5-30, 7-12, 8-15, 10-20, 15-25, 20-30, 25-45, or 30-50 in Patient Global Assessment of Psoriatic Arthritis. In some embodiments, the method achieves an improvement in Patient Global Assessment of Psoriatic Arthritis Pain. In some embodiments, the method provides an improvement of about 1, 2, 3, 4, 5, 10, 15, 20, 25, 30, 35, 40, 45, 50, or greater than 50; or about 1-5, 2-8, 2-15, 2-25, 3-9, 5-10, 5-20, 5-30, 7-12, 8-15, 10-20, 15-25, 20-30, 25-45, or 30-50 in Patient Global Assessment of Psoriatic Arthritis Pain. In some embodiments, the method achieves an improvement in Physician Global Assessment of Psoriatic Arthritis. In some embodiments, the method provides an improvement of about 1, 2, 3, 4, 5, 10, 15, 20, 25, 30, 35, 40, 45, 50, or greater than 50; or about 1-5, 2-8, 2-15, 2-25, 3-9, 5-10, 5-20, 5-30, 7-12, 8-15, 10-20, 15-25, 20-30, 25-45, or 30-50 in Physician Global Assessment of Psoriatic Arthritis. In some embodiments, the method achieves an improvement in Health Assessment Questionnaire-Disability Index (HAQ-DI) score. In some embodiments, the method achieves an improvement in dactylitis count. In some embodiments, the method achieves an improvement in Leed's Enthesitis Index. In some embodiments, the method achieves an improvement in Minimal Disease Activity. In some embodiments, the method achieves an improvement in Disease Activity Index for Psoriatic Arthritis (DAPSA). A DAPSA score of 5-14 represents a state of low disease activity. A score of <4 represents remission. Accordingly, in some embodiments, the method achieves a DAPSA score of 5-14, of 4, or of less than 4. In some embodiments, the method achieves an improvement in Psoriasis Area Severity Index (PASI)-75. In some embodiments, the method achieves an improvement in Psoriasis Area Severity Index (PASI)-75 after about 12 weeks of treatment, wherein the patient has ≥3% body surface area (BSA) psoriatic involvement at day 1 of treatment. In some embodiments, the method achieves a static Physician Global Assessment of 0 or 1 and at least a 2-point improvement from baseline, for example after about 12 weeks of treatment. In some embodiments, the improvement is achieved after 6 weeks of treatment. In some embodiments, the improvement is achieved after 18 weeks of treatment. In some embodiments, the improvement is achieved after 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks.

In some embodiments, the method achieves an improvement in circulating cytokines and/or an inflammatory biomarker, a 36-Item Short Form Health Survey, Functional Assessment of Chronic Illness—Fatigue, Disease Activity Score 28 with high-sensitivity C-Reactive Protein (hsCRP), Psoriatic Disease Activity Score at week 12 of treatment, Psoriatic Arthritis Response Criteria at week 12 of treatment, a mean decrease in psoriasis area severity index (PASI) of 25% or greater, or an improvement in BSA with psoriatic involvement at week 12, or an improvement in Physician Global Assessment of Psoriasis at week 12 of treatment.

In some embodiments, the method achieves an improvement in circulating cytokines and/or an inflammatory biomarker. In some embodiments, the biomarker comprises K16. In some embodiments, the biomarker comprises KRT16, IL17A, IL17F, or IL22 expression. In some embodiments, the method achieves an improvement in a 36-Item Short Form Health Survey as described herein. In some embodiments, the method achieves an improvement in Functional Assessment of Chronic Illness—Fatigue. In some embodiments, the method achieves an improvement in Disease Activity Score 28 with high-sensitivity C-Reactive Protein (hsCRP). In some embodiments, the method achieves an improvement in Psoriatic Disease Activity Score at week 12 of treatment. In some embodiments, the method achieves an improvement in Psoriatic Arthritis Response Criteria at week 12 of treatment. In some embodiments, the method achieves an improvement in PASI, e.g., at week 12 of treatment, among subjects with ≥3% BSA psoriatic involvement at day 1. In some embodiments, the method achieves an improvement in BSA with psoriatic involvement at Week 12 among subjects with ≥3% BSA psoriatic involvement at day 1. In some embodiments, the method achieves an improvement in Physician Global Assessment of Psoriasis, e.g., at week 12. In some embodiments, the improvement is achieved after 6 weeks of treatment. In some embodiments, the improvement is achieved after 18 weeks of treatment. In some embodiments, the improvement is achieved after 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks.

In one aspect, a method of inhibiting of interferon gamma (IFNγ) production in a patient, can include administering to a patient a therapeutically effective amount of Compound 1:

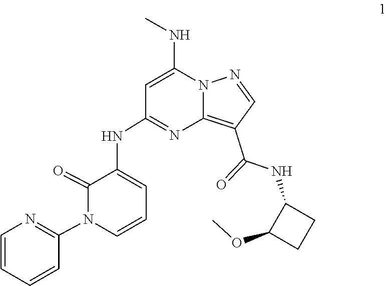 [SMILES: CNc1cc(Nc2cccn(-c3ccccn3)c2=O)nc2c(C(=O)N[C@@H]3CC[C@H]3OC)cnn12]

or a pharmaceutically acceptable salt thereof or a pharmaceutical composition thereof.

In some embodiments, the patient has psoriasis.

In some embodiments, the patient has psoriatic arthritis.

In some embodiments, the psoriasis is moderate to severe.

In some embodiments, the psoriatic arthritis is moderate to severe.

In some embodiments, a Tₘₐₓ of Compound 1 in plasma is achieved in about 3 hours to about 6 hours.

In some embodiments, a t_{1/2} of Compound 1 in plasma is achieved in about 17 hours to about 37 hours.

In some embodiments, Compound 1, or a pharmaceutically acceptable salt thereof, is administered at a dose of up to about 200 mg to the patient.

In some embodiments, Compound 1, or a pharmaceutically acceptable salt thereof, is administered at a dose of from about 20 mg to about 100 mg to the patient.

In some embodiments, Compound 1, or a pharmaceutically acceptable salt thereof, is administered at a dose of about 2 mg, about 5 mg, about 10 mg, about 15 mg, or about 30 mg to the patient.

In some embodiments, Compound 1, or a pharmaceutically acceptable salt thereof, is administered to the patient as a single dose.

In some embodiments, Compound 1, or a pharmaceutically acceptable salt thereof, is administered to the patient in multiple doses.

In some embodiments, Compound 1, or a pharmaceutically acceptable salt thereof, is administered to the patient daily for 2 weeks.

In some embodiments, Compound 1, or a pharmaceutically acceptable salt thereof, is administered to the patient daily for 4 weeks.

In some embodiments, Compound 1 is administered orally to the patient.

In some embodiments, the patient is a human.

In some embodiments, the patient has a PASI of at least about 12 prior to the administration.

In some embodiments, the patient has a PGA of at least about 3 prior to the administration.

In some embodiments, the patient has a BSA of at least about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, prior to the administration.

In some embodiments, the psoriasis does not comprise erythrodermic, pustular, predominantly guttate psoriasis, or drug-induced psoriasis.

In some embodiments, a serum Cₘₐₓ between about 25 ng/ml to about 50 ng/ml, about 50 ng/ml to about 100 ng/ml, about 100 ng/ml to about 125 ng/ml, about 125 ng/ml to about 150 ng/ml, about 150 ng/ml to about 175 ng/ml, about 175 ng/ml to about 200 ng/ml, about 200 ng/ml to about 225 ng/ml, about 225 ng/ml to about 250 ng/ml, about 250 ng/ml to about 275 ng/ml, or about 275 ng/ml to about 300 ng/ml, is achieved.

In one aspect, a method of treating moderate-to-severe plaque psoriasis in a patient in need thereof, can include administering a therapeutically effective amount of Compound 1:

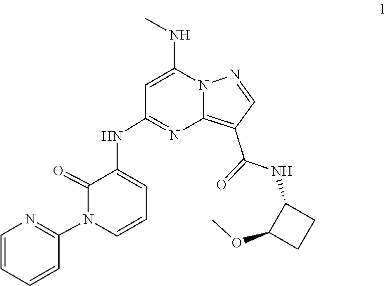 [SMILES: CNc1cc(Nc2cccn(-c3ccccn3)c2=O)nc2c(C(=O)N[C@@H]3CC[C@H]3OC)cnn12]

at a daily dose of from about 5 mg to about 30 mg.

In one aspect, Compound 1:

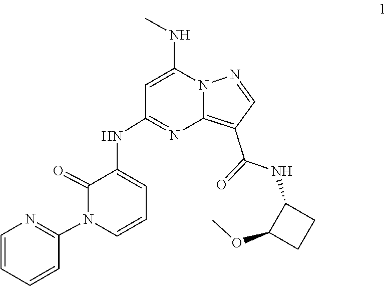 [SMILES: CNc1cc(Nc2cccn(-c3ccccn3)c2=O)nc2c(C(=O)N[C@@H]3CC[C@H]3OC)cnn12]

or a pharmaceutically acceptable salt thereof can be used in the treatment of psoriasis or psoriatic arthritis.

Compounds described herein include those described generally herein, and are further illustrated by the classes, subclasses, and species disclosed herein. As used herein, the following definitions shall apply unless otherwise indicated. For purposes described herein, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the term “Compound 1” refers to N-((1R,2R)-2-methoxycyclobutyl)-7-(methylamino)-5-((2-oxo-2H-[1,2′-bipyridin]-3-yl)amino)pyrazolo[1,5-a]pyrimidine-3-carboxamide having the formula:

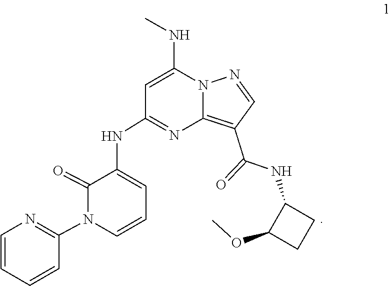 [SMILES: CNc1cc(Nc2cccn(-c3ccccn3)c2=O)nc2c(C(=O)N[C@@H]3CC[C@H]3OC)cnn12]

In some embodiments, Compound 1 or a pharmaceutically acceptable salt thereof, is in amorphous form. In some embodiments, Compound 1 or a pharmaceutically acceptable salt thereof, is in crystalline form.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms indcpcndcntly sclcctcd from nitrogcn, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

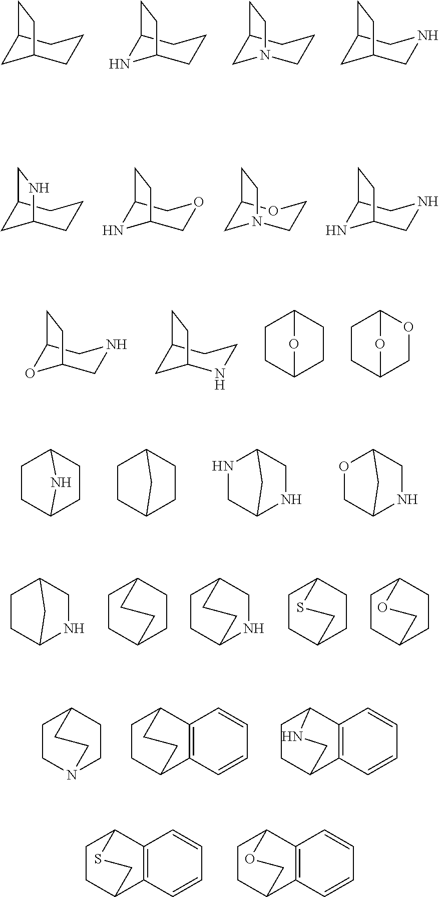

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned and described herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}. —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, =NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms indepcndcntly selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C_{1-_}aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†t}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms. In certain embodiments, Ring B of a provided compound may be substituted with one or more deuterium atoms.

As used herein, the term “inhibitor” is defined as a compound that binds to and/or inhibits TYK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

The terms “measurable affinity” and “measurably inhibit,” as used herein, means a measurable change in a TYK2 protein kinase activity between a sample comprising a compound described herein, or composition thereof, and a TYK2 protein kinase, and an equivalent sample comprising an TYK2 protein kinase, in the absence of said compound, or composition thereof.

“Dermatology Life Quality Index,” as used herein, or DLQI, is a simple 10-question validated questionnaire that has been used in more than 40 different skin conditions. See Finlay A Y, Khan G K. “Dermatology Life Quality Index (DLQI)—a simple practical measure for routine clinical use,” Clin Exp Dermatol. 1994; 19(3):210-216, the entire disclosure of which is hereby incorporated by reference in its entirety.

“Body surface area,” as used herein, refers to the % of surface area that is covered with psoriasis. A reduce in “percentage points” refers to an amount by which said % surface area is reduced after treatment. For example, a 7-percentage point reduction would describe a reduction from a BSA of 57% to a BSA of 50%.

A “Numeric Rating Scale,” (NRS), as used herein, refers to a scale of 1 to 10, wherein the symptom (e.g., pain or pruritis) is scored by a patient wherein 10 is the worst affliction imaginable and 0 is no affliction at all.

“Cₘₐₓ” as used herein, is the maximum (or peak) serum concentration that a drug achieves in a specified compartment or test area of the body after the drug has been administered and before the administration of a second dose.

“SDD,” as used herein, refers to a pharmaceutical formulation (e.g., of Compound 1 or a pharmaceutically acceptable salt thereof) which is a spray dried formulation. The formulation may comprise a compound of the disclosure and hypromellose acetate succinate (HPMCAS). In one embodiment, the HMPCAS is HPMCAS-M, wherein the “M” indicates (acetyl content 7.0% to 11.0%, succinoyl content 10% to 14%). The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See European Patent Application Nos. 0 901 786, 1 027 886, 1 027 887, 1 027 888, and PCT Application Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

“TPGS” or “Vitamin E TPGS” as a descriptor for a pharmaceutical formulation for a compound of the disclosure, as used herein, refers to a pharmaceutical formulation (e.g., of Compound 1 or a pharmaceutically acceptable salt thereof) which includes the components of (a) the active compound; (b) one or more diluents (e.g., microcrystalline cellulose); (c) one or more solubilizers (e.g., D-α-tocopherol polyethylene glycol succinate [Vitamin E TPGS]); and (d) one or more binders (e.g., povidone). The formulation may be prepared using granulation processes (e.g., wet granulation). “Granulation,” as used herein, refers to a process to produce larger or smaller granules or particles of a substance or mixture of substances. The process also may remove fine granules and improve flowability within the formulation. Both wet granulation and/or dry granulation may be employed. Dry granulation is achieved using only a combination of granules without the need for any liquid thereon. Slugging uses a tablet press to form large tablets that vary in weight due to the poor flowability of the formulation. The slugs created are then put through a granulator to be broken down into granules and then compressed once again for a final granulated product.

### 3. Exemplary Compounds:

According to one aspect, a method for treating inflammatory disorders (e.g., psoriasis or psoriatic arthritis) in a patient in need thereof, can include administering a therapeutically effective amount of a TYK2 inhibitor (e.g., Compound 1), or a pharmaceutically acceptable salt thereof. In some embodiments, the method comprises administering up to 100 mg of a TYK2 inhibitor (e.g., Compound 1), or a pharmaceutically acceptable salt thereof, in single or multiple (e.g., divided) doses.

U.S. Pat. No. 11,046,698, the entirety of which is hereby incorporated herein by reference, describes certain therapeutically beneficial compounds. Such compounds include Compound 1. Compound 1 is designated as I-908 in U.S. Pat. No. 11,046,698 and its synthesis, properties, and use in treating various diseases and disorders is described in detail therein. Numerous other TYK2 inhibitors are described in U.S. Pat. No. 11,046,698 which may be used in a method of the present disclosure. Accordingly, in some embodiments, the TYK2 inhibitor is one of those described in U.S. Pat. No. 11,046,698.

In certain embodiments, a TYK2 inhibitor for use in a disclosed method is of formula I:

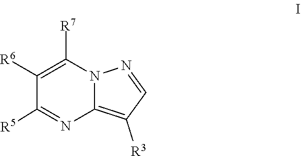

or a pharmaceutically acceptable salt thereof, wherein:
R³ is —C(O)NH₂; —C(O)NHR^{3A}; —C(O)N(R^{3A})₂; or a 5-6 membered monocyclic heteroaryl ring having 1-4 heteroatoms independently selected from nitrogen, oxygen, and sulfur, wherein said ring is substituted with m instances of R^{5B};
R⁵ is hydrogen, or -L¹-R^{5A};
R⁶ is hydrogen, R^{A}, or R^{B};
or R⁵ and R⁶ are taken together with their intervening atoms to form a 4-7 membered partially unsaturated, or heteroaryl ring having 0-3 heteroatoms independently selected from nitrogen, oxygen, and sulfur, wherein said ring is substituted by R^{5A} and n instances of R^{C};
R⁷ is hydrogen, halogen, —NH₂, —NHR^{7A}, or —NHC(O)R^{7A};
or R⁶ and R⁷ are taken together with their intervening atoms to form a 4-7 membered partially unsaturated, or heteroaryl ring having 0-3 heteroatoms independently selected from nitrogen, oxygen, and sulfur; wherein said ring is substituted by p instances of R^{C};
L¹ is a covalent bond or a C₁₋₄ bivalent saturated or unsaturated, straight or branched hydrocarbon chain wherein one or two methylene units of the chain are optionally and independently replaced by —C(R^{5B})₂—, —CH(R^{5B})—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
R^{3A} and R^{7A} are each independently R^{B}, and are each substituted by q instances of R^{C};
R^{5A} and each instance of R^{5B} are each independently R^{A} or R^{B}, and are each substituted by r instances of R^{C};
each instance of R^{A} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R;
each instance of R^{B} is independently C₁₋₆ aliphatic; phenyl; a 5-6 membered monocyclic heteroaryl ring having 1-4 heteroatoms independently selected from nitrogen, oxygen, and sulfur, an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{C} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
wherein each hydrogen bound to carbon can be optionally and independently replaced by deuterium; and
each instance of m, n, p, q, and r is independently 0, 1, 2, 3, or 4.

In some embodiments, the TYK2 inhibitor for use in a disclosed method is Compound 1a:

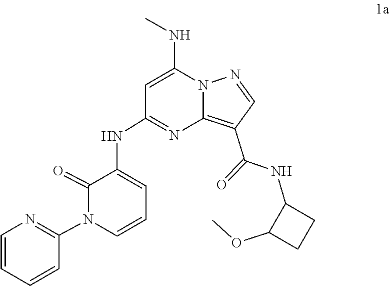 [SMILES: CNc1cc(Nc2cccn(-c3ccccn3)c2=O)nc2c(C(=O)NC3CCC3OC)cnn12]

or a pharmaceutically acceptable salt thereof or a pharmaceutical composition thereof.

In some embodiments, the TYK2 inhibitor for use in a disclosed method is Compound 1:

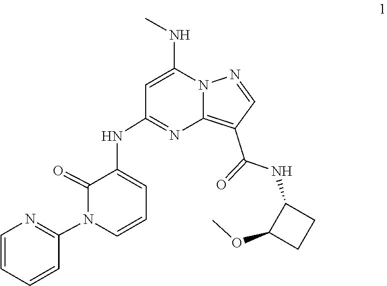 [SMILES: CNc1cc(Nc2cccn(-c3ccccn3)c2=O)nc2c(C(=O)N[C@@H]3CC[C@H]3OC)cnn12]

or a pharmaceutically acceptable salt thereof or a pharmaceutical composition thereof.

Compounds and compositions described herein are generally useful for the inhibition of kinase activity of one or more enzymes. In some embodiments the kinase inhibited by the compounds and methods described herein is TYK2.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g. IFN-α, IFN-β, IFN-κ, IFN-δ, IFN—ε, IFN-τ, IFN-ω, and IFN-ζ (also known as limitin), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signaling pathway,” Cell (1992) 70:313; Stahl et al., “Association and activation of Jak-Tyk kinases by CNTF-LIF-OSM-IL-6β receptor components,” Science (1994) 263:92; Finbloom et al., “IL-10 induces the tyrosine phosphorylation of Tyk2 and Jak1 and the differential assembly of Stat1 and Stat3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoietic cells: similarities and differences in signal transduction with interleukin-4 and insulin,” J. Biol. Chem. (1995) 270:12286; Parham et al., “A receptor for the heterodimeric cytokine IL-23 is composed of IL-12Rβ1 and a novel cytokine receptor subunit, IL-23R,” J. Immunol. (2002) 168:5699. The activated TYK2 then goes on to phosphorylate further signaling proteins such as members of the STAT family, including STAT1, STAT2, STAT4, and STAT6.

TYK2 activation by IL-23 has been linked to inflammatory bowel disease (IBD), Crohn's disease, and ulcerative colitis. Duerr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behçet's disease. Cho et al., “Genomics and the multifactorial nature of human auto-immune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MHC class I, IL10, and IL23R-IL12RB2 regions associated with Behçet's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAPI,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11.

Decreased TYK2 activity leads to protection of joints from collagen antibody-induced arthritis, a model of human rheumatoid arthritis. Mechanistically, decreased TYK2 activity reduced the production of Tₕ1/Tₕ17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupus Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association of NCF2, IKZF1, IRF8, IFiH1, and TYK2 with Systemic Lupus Erythematosus,” PLoS Genetics (2011) 7(10):e1002341.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects. Simma et al. “Identification of an Indispensable Role for Tyrosine Kinase 2 in CTL-Mediated Tumor Surveillance,” Cancer Res. (2009) 69:203-211.

However, paradoxically to the decreased tumor surveillance observed by Simma et al., studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective inhibition of TYK2 has been suggested as a suitable target for patients with IL-10 and/or BCL2-addicted tumors, such as 70% of adult T-cell leukemia cases. Fontan et al. “Discovering What Makes STAT Signaling TYK in T-ALL,” Cancer Disc. (2013) 3:494-496.

TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in postmortem brains of Alzheimer's patients. Wan et al. “Tyk/STAT3 Signaling Mediates β-Amyloid-Induced Neuronal Cell Death: Implications in Alzheimer's Disease,” J. Neurosci. (2010) 30(20):6873-6881.

Inhibition of JAK-STAT signaling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signaling promotes hair growth,” Sci. Adv. (2015) 1(9):e1500973.

Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, inhibitors of TYK2 can show selectivity over JAK2.

The activity of a compound described herein as an inhibitor of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 inhibitor include those described and disclosed in, e.g., each of which is herein incorporated by reference in its entirety. Detailed conditions for assaying a compound described herein as an inhibitor of TYK2, or a mutant thereof, are set forth in the Examples below and in U.S. Pat. No. 11,046,698, which is hereby incorporated by reference.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of TYK2 and are therefore useful for treating one or more disorders associated with activity of TYK2 or mutants thereof. Thus, in certain embodiments, a method for treating a TYK2-mediated disorder can include the step of administering to a patient in need thereof a compound described herein, or pharmaceutically acceptable composition thereof.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

In some embodiments, a method for treating one or more disorders, wherein the disorders are selected from autoimmune disorders, inflammatory disorders, proliferative disorders, endocrine disorders, neurological disorders, and disorders associated with transplantation, said method can include administering to a patient in need thereof, a pharmaceutical composition comprising an effective amount of a compound described herein, or a pharmaceutically acceptable salt thereof.

In some embodiments, the disorder is an autoimmune disorder. In some embodiments the disorder is selected from type 1 diabetes, cutaneous lupus erythematosus, systemic lupus erythematosus, multiple sclerosis, psoriasis (e.g., plaque psoriasis), Behçet's disease, POEMS syndrome, Crohn's disease, ulcerative colitis, and inflammatory bowel disease.

In some embodiments, the disorder is an inflammatory disorder. In some embodiments, the inflammatory disorder is rheumatoid arthritis, asthma, chronic obstructive pulmonary disease, psoriasis (e.g., plaque psoriasis), psoriatic arthritis, hepatomegaly, Crohn's disease, ulcerative colitis, or inflammatory bowel disease.

In some embodiments, a method of treating psoriasis (e.g., plaque psoriasis) or psoriatic arthritis in a patient in need thereof, can include administering a TYK2 inhibitor (e.g., Compound 1) described herein, or a pharmaceutically acceptable salt thereof.

In some embodiments, a method of treating psoriasis (e.g., plaque psoriasis) in a patient in need thereof, can include administering a TYK2 inhibitor (e.g., Compound 1) described herein, or a pharmaceutically acceptable salt thereof.

In some embodiments, a method of treating psoriatic arthritis in a patient in need thereof, can include administering a TYK2 inhibitor (e.g., Compound 1) described herein, or a pharmaceutically acceptable salt thereof.

In some embodiments, the disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signaling. In some embodiments the disorder is associated with type I interferon signaling. In some embodiments the disorder is associated with IL-10 signaling. In some embodiments the disorder is associated with IL-12 signaling. In some embodiments the disorder is associated with IL-23 signaling.

Compounds described herein are also useful in the treatment of inflammatory or allergic conditions of the skin, for example psoriasis (e.g., plaque psoriasis), contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, cutaneous lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds described herein may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minimal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods described herein is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods described herein is a Tₕ1 or Tₕ17 mediated disease. In some embodiments the Tₕ17 mediated disease is selected from cutaneous lupus erythematosus, Systemic lupus erythematosus, Multiple sclerosis, and inflammatory bowel disease (including Crohn's disease or ulcerative colitis).

In some embodiments the inflammatory disease which can be treated according to the methods described herein is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, the invention can provide the use of a compound according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

Without wishing to be bound by any particular theory, it is believed that proximity of an inhibitor compound, or pendant moiety of an inhibitor compound, to the water of interest facilitates displacement or disruption of that water by the inhibitor compound, or pendant moiety of an inhibitor compound. In some embodiments, a water molecule displaced or disrupted by an inhibitor compound, or pendant moiety of an inhibitor compound, is an unstable water molecule.

In certain embodiments, the method employs a complex comprising TYK2 and an inhibitor, wherein at least one unstable water of TYK2 is displaced or disrupted by the inhibitor. In some embodiments, at least two unstable waters selected are displaced or disrupted by the inhibitor.

### 5. Pharmacokinetics/Pharmacodynamics

In some embodiments, the present disclosure provides a method of administering a TYK2 inhibitor (e.g., Compound 1) to a patient in need thereof, comprising administering to said patient a therapeutically effective amount of Compound 1, or a pharmaceutically acceptable salt thereof, or composition thereof, wherein certain pharmacokinetic parameters are achieved. In some embodiments, the disclosed methods and uses of administering Compound 1 achieving certain pharmacokinetic parameters of the disclosure has advantages in treating the diseases described herein, such as inflammatory disorders. Such diseases include psoriasis and psoriatic arthritis.

In some embodiments, a Tₘₐₓ of a TYK2 inhibitor (e.g., Compound 1) in plasma is achieved in up to about 10 hours, for example, about 1 hour, about 2 hours, about 3 hours, about 4 hours, about 5 hours, 6 hours, about 7 hours, about 8 hours, about 9 hours, or about 10 hours, or any range of time created by using two of the aforementioned times as endpoints. In some embodiments, a Tₘₐₓ is achieved in about 1 to about 10 hours, about 1 to about 9 hours, about 2 to about 10 hours, about 2 to about 9 hours, about 3 to about 10 hours, about 3 to about 9 hours, about 4 to about 10 hours, about 4 to about 9 hours, about 5 to about 10 hours, about 5 to about 9 hours, about 6 to about 10 hours, about 6 to about 9 hours, about 7 to about 10 hours, about 7 to about 9 hours, about 8 to about 10 hours, about 9 to about 10 hours, or about 8 to about 9 hours. In some embodiments, a Tₘₐₓ of a TYK2 inhibitor (e.g., Compound 1) in plasma is achieved in up to about 3 hours. In some embodiments, a Tₘₐₓ of a TYK2 inhibitor (e.g., Compound 1) in plasma is achieved in up to about 4 hours. In some embodiments, a Tₘₐₓ of Compound 1 in plasma is achieved in up to about 5 hours. In some embodiments, a Tₘₐₓ of a TYK2 inhibitor (e.g., Compound 1) in plasma is achieved in up to about 6 hours.

In some embodiments, a Tₘₐₓ of a TYK2 inhibitor (e.g., Compound 1) in plasma is achieved from about 1 hour to about 4 hours, from about 2 hours to about 5 hours, from about 3 hours to about 6 hours, from about 4 hours to about 7 hours, from about 5 hours to about 8 hours, from about 6 hours to about 9 hours, or from about 7 hours to about 10 hours. In some embodiments, a Tₘₐₓ of a TYK2 inhibitor (e.g., Compound 1) in plasma is achieved from about 3 hours to about 6 hours.

In some embodiments, a t_{1/2} of a TYK2 inhibitor (e.g., Compound 1) in plasma is achieved in up to about 50 hours, for example, about 10 hours, about 11 hours, about 12 hours, about 13 hours, about 14 hours, about 15 hours, 16 hours, about 17 hours, about 18 hours, about 19 hours, about 20 hours, about 21 hours, about 22 hours, about 23 hours, about 24 hours, about 25 hours, 26 hours, about 27 hours, about 28 hours, about 29 hours, about 30 hour, about 31 hour, about 32 hours, about 33 hours, about 34 hours, about 35 hours, 36 hours, about 37 hours, about 38 hours, about 39 hours, about 40 hour, about 41 hour, about 42 hours, about 43 hours, about 44 hours, about 45 hours, 46 hours, about 47 hours, about 48 hours, about 49 hours, or about 50 hours, or any range of time created by using two of the aforementioned times as endpoints. In some embodiments, a t_{1/2} of a TYK2 inhibitor (e.g., Compound 1) in plasma is achieved in up to about 17 hours. In some embodiments, a t_{1/2} of a TYK2 inhibitor (e.g., Compound 1) in plasma is achieved in up to about 18 hours. In some embodiments, a t_{1/2} of Compound 1 in plasma is achieved in up to about 19 hours. In some embodiments, a t_{1/2} of a TYK2 inhibitor (e.g., Compound 1) in plasma is achieved in up to about 20 hours. In some embodiments, a t_{1/2} of a TYK2 inhibitor (e.g., Compound 1) in plasma is achieved in up to about 21 hours. In some embodiments, a t_{1/2} of a TYK2 inhibitor (e.g., Compound 1) in plasma is achieved in up to about 22 hours. In some embodiments, a t_{1/2} of Compound 1 in plasma is achieved in up to about 23 hours. In some embodiments, a t_{1/2} of a TYK2 inhibitor (e.g., Compound 1) in plasma is achieved in up to about 24 hours. In some embodiments, a t_{1/2} of a TYK2 inhibitor (e.g., Compound 1) in plasma is achieved in up to about 25 hours. In some embodiments, a t_{1/2} of a TYK2 inhibitor (e.g., Compound 1) in plasma is achieved in up to about 26 hours. In some embodiments, a t_{1/2} of Compound 1 in plasma is achieved in up to about 27 hours. In some embodiments, a t_{1/2} of a TYK2 inhibitor (e.g., Compound 1) in plasma is achieved in up to about 28 hours. In some embodiments, a t_{1/2} of a TYK2 inhibitor (e.g., Compound 1) in plasma is achieved in up to about 29 hours. In some embodiments, a t_{1/2} of a TYK2 inhibitor (e.g., Compound 1) in plasma is achieved in up to about 30 hours. In some embodiments, a t_{1/2} of Compound 1 in plasma is achieved in up to about 31 hours. In some embodiments, a t_{1/2} of a TYK2 inhibitor (e.g., Compound 1) in plasma is achieved in up to about 32 hours. In some embodiments, a t_{1/2} of a TYK2 inhibitor (e.g., Compound 1) in plasma is achieved in up to about 33 hours. In some embodiments, a t_{1/2} of a TYK2 inhibitor (e.g., Compound 1) in plasma is achieved in up to about 34 hours. In some embodiments, a t_{1/2} of Compound 1 in plasma is achieved in up to about 35 hours. In some embodiments, a t_{1/2} of a TYK2 inhibitor (e.g., Compound 1) in plasma is achieved in up to about 36 hours. In some embodiments, a t_{1/2} of a TYK2 inhibitor (e.g., Compound 1) in plasma is achieved in up to about 37 hours.

In some embodiments, a t_{1/2} of a TYK2 inhibitor (e.g., Compound 1) in plasma is achieved from about 10 hours to 30 hours, from about 12 hours to 32 hours, from about 14 hours to 34 hours, from about 16 hours to 36 hours, from about 18 hours to 38 hours, from about 20 hours to 40 hours, or from about 22 hours to 42 hours. In some embodiments, a t_{1/2} of a TYK2 inhibitor (e.g., Compound 1) in plasma is achieved from about 17 hours to 37 hours.

In some embodiments, the present disclosure provides a method of administering a TYK2 inhibitor (e.g., Compound 1) to a patient in need thereof, comprising administering to said patient a therapeutically effective amount of a TYK2 inhibitor (e.g., Compound 1), or a pharmaceutically acceptable salt thereof, or pharmaceutical composition thereof, wherein certain pharmacodynamic results are achieved.

In some embodiments of the methods and uses disclosed herein, the administration of a TYK2 inhibitor (e.g., Compound 1) leads to rapid inhibition of interferon gamma (IFNγ) production and increasing exposure correlated with the increased inhibition of IFNγ. In some embodiments, a method of inhibiting of IFNγ production in a patient, can include administering to the patient a TYK2 inhibitor (e.g., Compound 1) described herein, or a pharmaceutically acceptably salt thereof or pharmaceutical composition thereof.

In some embodiments, the disclosed methods and uses of administering Compound 1 include a mean decrease in psoriasis area severity index (PASI) of up to about 100% or up to about 90%, for example, up to about 20%, about 25%, about 30%, about 35%, about 40%, about 45%, about 50%, about 55%, about 60%, about 65%, about 70%, about 75%, about 80%, about 85%, or about 90%, or any range of % created by using two of the aforementioned % as endpoints. A PASI score is a tool used to measure the severity and extent of psoriasis and is well known to those having ordinary skill in the art. In some embodiments, a mean decrease in psoriasis area severity index (PASI) of up to about 50% is achieved. In some embodiments, a mean decrease in psoriasis area severity index (PASI) of up to about 75% is achieved. In some embodiments, a mean decrease in psoriasis area severity index (PASI) of up to about 90% is achieved. In some embodiments, a mean decrease in psoriasis area severity index (PASI) of up to about 100% is achieved. In some embodiment, the mean decrease in psoriasis area severity index (PASI) of about 30%, about 47%, or about 48%, is achieved.

In some embodiments, the disclosed methods and uses provide a mean decrease in PASI of at least 25% is achieved. In some embodiments, the disclosed methods and uses provide a mean decrease in PASI of at least 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, or greater is achieved. In some embodiments, the disclosed methods and uses provide a mean decrease in PASI of about 26-65%, 26-60%, 26-55%, 26-50%, 26-45%, 26-40%, 26-35%, 26-30%, 27-65%, 27-65%, 27-60%, 27-55%, 27-50%, 27-45%, 27-40%, 27-35%, 27-30%, 28-65%, 28-65%, 28-60%, 28-55%, 28-50%, 28-45%, 28-40%, 28-35%, 28-30%, 29-65%, 29-65%, 29-60%, 29-55%, 29-50%, 29-45%, 29-40%, 29-35%, 30-65%, 30-60%, 30-55%, 30-50%, 30-45%, 30-40%, 30-35%, 35-65%, 35-60%, 35-55%, 35-50%, 35-45%, 35-40%, 40-65%, 40-60%, 40-55%, 40-50%, 40-45%, 45-65%, 45-60%, 45-55%, 45-50%, 50-65%, 50-65%, 50-60%, 50-55%, 55-65%, or 55-60% is achieved.

In some embodiments, a mean decrease in psoriasis area severity index (PASI) of about 50% to 75% is achieved. In some embodiments, a mean decrease in psoriasis area severity index (PASI) of about 75% to 100% is achieved.

In some embodiments, the disclosed methods and uses provide a statistically significant (p<0.05) improvement in PASI across a group of patients having an inflammatory disease such as psoriasis or psoriatic arthritis.

In some embodiments, the patient has a PASI of at least about 12 prior to the administration. In some embodiments, the patient has a PASI of about 10 to about 20 prior to the administration. In some embodiments, the patient has a PASI of about 20 to about 30 prior to the administration. In some embodiments, the patient has a PASI of about 30 to about 40 prior to the administration. In some embodiments, the patient has a PASI of about 40 to about 50 prior to the administration. In some embodiments, the patient has a PASI of about 50 to about 60 prior to the administration. In some embodiments, the patient has a PASI of about 60 to about 72 prior to the administration.

In some embodiments, a Physician Global Assessment (PGA) of 0, 1, or 2 is achieved. In some embodiments, a Physician Global Assessment (PGA) of 0 or 1 is achieved. In some embodiments, a Physician Global Assessment (PGA) of 0 is achieved.

In some embodiments, the patient has a PGA of 1 prior to the administration. In some embodiments, the patient has a PGA of 2 prior to the administration. In some embodiments, the patient has a PGA of 3 prior to the administration. In some embodiments, the patient has a PGA of 4 prior to the administration. In some embodiments, the patient has a PGA of 5 prior to the administration.

In some embodiments, a Dermatology Life Quality Index (DLQI) score is reduced by an amount between about 1 to about 3, about 3 to about 6, about 6 to about 9, about 9 to about 12, about 12 to about 15, about 15 to about 18, about 18 to about 21, about 21 to about 24, about 24 to about 27, or about 27 to about 30. In some embodiments, a Dermatology Life Quality Index (DLQI) score is reduced by an amount between about 1 to about 3. In some embodiments, a Dermatology Life Quality Index (DLQI) score is reduced by an amount between about 3 to about 6. In some embodiments, a Dermatology Life Quality Index (DLQI) score is reduced by an amount between about 6 to about 9. In some embodiments, a Dermatology Life Quality Index (DLQI) score is reduced by an amount between about 9 to about 12. In some embodiments, a Dermatology life Quality Index (DLQI) score is reduced by an amount between about 12 to about 15. In some embodiments, a Dermatology life Quality Index (DLQI) score is reduced by an amount between about 15 to about 18. In some embodiments, a Dermatology Life Quality Index (DLQI) score is reduced by an amount between about 18 to about 21. In some embodiments, a Dermatology Life Quality Index (DLQI) score is reduced by an amount between about 21 to about 24. In some embodiments, a Dermatology life Quality Index (DLQI) score is reduced by an amount between about 24 to about 27. In some embodiments, a Dermatology life Quality Index (DLQI) score is reduced by an amount between about 27 to about 30.

In some embodiments, the patient's Body Surface Area (BSA) affected by psoriasis is reduced by a number of percentage points between about 1 to about 10, about 10 to about 20, about 20 to about 30, about 30 to about 40, about 40 to about 50, about 50 to about 60, about 60 to about 70, about 70 to about 80, about 80 to about 90, or about 90 to about 100. In some embodiments, the patient's Body Surface Area (BSA) affected by psoriasis is reduced by a number of percentage points between about 1 to about 10. In some embodiments, the patient's Body Surface Area (BSA) is reduced by a number of percentage points between about 10 to about 20. In some embodiments, the patient's Body Surface Area (BSA) is reduced by a number of percentage points between about 20 to about 30. In some embodiments, the patient's Body Surface Area (BSA) is reduced by a number of percentage points between about 30 to about 40. In some embodiments, the patient's Body Surface Area (BSA) is reduced by a number of percentage points between about 40 to about 50. In some embodiments, the patient's Body Surface Area (BSA) is reduced by a number of percentage points between about 50 to about 60. In some embodiments, the patient's Body Surface Area (BSA) is reduced by a number of percentage points between about 60 to about 70. In some embodiments, the patient's Body Surface Area (BSA) is reduced by a number of percentage points between about 70 to about 80. In some embodiments, the patient's Body Surface Area (BSA) is reduced by a number of percentage points between about 80 to about 90. In some embodiments, the patient's Body Surface Area (BSA) is reduced by a number of percentage points between about 90 to about 100.

In some embodiments, the patient has a BSA affected by psoriasis of at least about 10%, for example, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, prior to the administration. In some embodiments, the patient has an affected BSA of about 10% to about 20% prior to the administration. In some embodiments, the patient has a BSA of about 20% to about 30% prior to the administration. In some embodiments, the patient has a BSA of about 30% to about 40% prior to the administration. In some embodiments, the patient has a BSA of about 40% to about 50% prior to the administration. In some embodiments, the patient has a BSA of about 50% to about 60% prior to the administration. In some embodiments, the patient has a BSA of about 60% to about 70% prior to the administration. In some embodiments, the patient has a BSA of about 70% to about 80% prior to the administration. In some embodiments, the patient has a BSA of about 80% to about 90% prior to the administration. In some embodiments, the patient has a BSA of about 90% to about 100% prior to the administration.

In some embodiments, a reduction in pain numeric rating scale (NRS) of about 1, 2, 3, 4, 5, 6, 7, 8, 9, or 10 is achieved. In some embodiments, a reduction in pain numeric rating scale (NRS) of about 1 is achieved. In some embodiments, a reduction in pain numeric rating scale (NRS) of about 2 is achieved. In some embodiments, a reduction in pain numeric rating scale (NRS) of about 3 is achieved. In some embodiments, a reduction in pain numeric rating scale (NRS) of about 4 is achieved. In some embodiments, a reduction in pain numeric rating scale (NRS) of about 5 is achieved. In some embodiments, a reduction in pain numeric rating scale (NRS) of about 6 is achieved. In some embodiments, a reduction in pain numeric rating scale (NRS) of about 7 is achieved. In some embodiments, a reduction in pain numeric rating scale (NRS) of about 8 is achieved. In some embodiments, a reduction in pain numeric rating scale (NRS) of about 9 is achieved. In some embodiments, a reduction in pain numeric rating scale (NRS) of about 10 is achieved.

In some embodiments, a reduction in pruritus numeric rating scale (NRS) of about 1, 2, 3, 4, 5, 6, 7, 8, 9, or 10 is achieved. In some embodiments, a reduction in pruritus numeric rating scale (NRS) of about 1 is achieved. In some embodiments, a reduction in pruritus numeric rating scale (NRS) of about 2 is achieved. In some embodiments, a reduction in pruritus numeric rating scale (NRS) of about 3 is achieved. In some embodiments, a reduction in pruritus numeric rating scale (NRS) of about 4 is achieved. In some embodiments, a reduction in pruritus numeric rating scale (NRS) of about 5 is achieved. In some embodiments, a reduction in pruritus numeric rating scale (NRS) of about 6 is achieved. In some embodiments, a reduction in pruritus numeric rating scale (NRS) of about 7 is achieved. In some embodiments, a reduction in pruritus numeric rating scale (NRS) of about 8 is achieved. In some embodiments, a reduction in pruritus numeric rating scale (NRS) of about 9 is achieved. In some embodiments, a reduction in pruritus numeric rating scale (NRS) of about 10 is achieved.

In some embodiments, an inhibition of IL-12/18-induced IFNγ production between about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, is achieved. In some embodiments, an inhibition of IL-12/18-induced IFNγ production between about 1% to about 10% is achieved. In some embodiments, an inhibition of IL-12/18-induced IFNγ production between about 10% to about 20% is achieved. In some embodiments, an inhibition of IL-12/18-induced IFNγ production between about 20% to about 30% is achieved. In some embodiments, an inhibition of IL-12/18-induced IFNγ production of about 30% to about 40% is achieved. In some embodiments, an inhibition of IL-12/18-induced IFNγ production between about 40% to about 50% is achieved. In some embodiments, an inhibition of IL-12/18-induced IFNγ production between about 50% to about 60% is achieved. In some embodiments, an inhibition of IL-12/18-induced IFNγ production between about 60% to about 70% is achieved. In some embodiments, an inhibition of IL-12/18-induced IFNγ production between about 70% to about 80% is achieved. In some embodiments, an inhibition of IL-12/18-induced IFNγ production between about 80% to about 90% is achieved. In some embodiments, an inhibition of IL-12/18-induced IFNγ production between about 90% to about 100% achieved.

In some embodiments, the psoriasis does not comprise erythrodermic, pustular, predominantly guttate psoriasis, or drug-induced psoriasis.

In some embodiments, a serum Cₘₐₓ between about 25 ng/ml to about 50 ng/ml, about 50 ng/ml to about 100 ng/ml, about 100 ng/ml to about 125 ng/ml, about 125 ng/ml to about 150 ng/ml, about 150 ng/ml to about 175 ng/ml, about 175 ng/ml to about 200 ng/ml, about 200 ng/ml to about 225 ng/ml, about 225 ng/ml to about 250 ng/ml, about 250 ng/ml to about 275 ng/ml, or about 275 ng/ml to about 300 ng/ml, is achieved. In some embodiments, a serum Cₘₐₓ between about 25 ng/ml to about 50 ng/ml is achieved. In some embodiments, a serum Cₘₐₓ between about 50 ng/ml to about 75 ng/ml is achieved. In some embodiments, a serum Cₘₐₓ between about 75 ng/ml to about 100 ng/ml is achieved. In some embodiments, a serum Cₘₐₓ between about 100 ng/ml to about 125 ng/ml is achieved. In some embodiments, a serum Cₘₐₓ between about 125 ng/ml to about 175 ng/ml is achieved. In some embodiments, a scrum Cₘₐₓ between about 175 ng/ml to about 200 ng/ml is achieved. In some embodiments, a serum Cₘₐₓ between about 200 ng/ml to about 225 ng/ml is achieved. In some embodiments, a serum Cₘₐₓ between about 225 ng/ml to about 250 ng/ml is achieved. In some embodiments, a serum Cₘₐₓ between about 250 ng/ml to about 300 ng/ml is achieved.

### 6. Dosing and Schedules

In some embodiments, the methods and uses described herein, such as the method or use in treating inflammatory disorders (e.g., psoriasis and psoriatic arthritis) in a patient in need thereof, is achieved by administering (e.g., orally) a therapeutically effective amount of a TYK2 inhibitor (e.g., Compound 1), such as up to 200 mg in a single or multiple doses. In some embodiments, the TYK2 inhibitor is administered one, two, three, four, five, six, seven, or eight times daily. In some embodiments, the method comprises administering (e.g., orally), in a single or multiple doses ranging from about 2 to about 200 mg/dosage, such as about 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 145 mg, 150 mg, 160 mg, 170 mg, 185 mg, 190 mg, 195 mg, or about 200 mg, or any range of amounts created by using two of the aforementioned amounts as endpoints. For example, an oral dosage can include 2 mg, 5 mg, 10 mg, 15 mg, 20 mg, 25 mg, 30 mg, 35 mg, 40 mg, 45 mg, or 50 mg/dosage form of a TYK2 inhibitor (e.g., Compound 1) or a pharmaceutically acceptable salt thereof.

In some embodiments, the method comprises administering (e.g., orally), in a single or multiple doses ranging from about 2 to about 200 mg/dosage, such as about 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, 145 mg, 150 mg, 160 mg, 170 mg, 185 mg, 190 mg, 195 mg, or about 200 mg daily.

In some embodiments, the method comprises administering (e.g., orally), in a single or multiple doses ranging from about 2 to about 200 mg, about 2 mg to about 10 mg, about 15 mg to about 20 mg, about 25 mg to about 30 mg, about 35 mg to about 40 mg, about 45 mg to about 50 mg, about 60 mg to about 70 mg, about 80 mg to about 90 mg, about 100 mg to about 110 mg, about 120 mg to about 130 mg, about 140 mg to about 150 mg, about 150 mg to about 160 mg, about 170 mg to about 185 mg, about 190 mg to about 200 mg, about 5 mg to about 30 mg, about 10 mg to about 30 mg, about 10 mg to about 50 mg, about 10 mg to about 100 mg, about 25 mg to about 50 mg, about 25 mg to about 100 mg, about 25 mg to about 200 mg, about 30 mg to about 50 mg, about 30 mg to about 100 mg, about 40 mg to about 50 mg, about 40 mg to about 100 mg, about 50 mg to about 100 mg, about 75 mg to about 100 mg, about 75 mg to about 200 mg, about 100 mg to about 200 mg, about 125 mg to about 150 mg, or about 150 to about 200 mg daily. Any specific dosage amount in between each of the ranges of this paragraph is contemplated. For example, for the recitation of “about 2 mg to about 10 mg,” this encompasses administration of about 2 mg, about 3 mg, about 4 mg, about 5 mg, about 6 mg, about 7 mg, about 8 mg, about 9 mg, or about 10 mg. Also contemplated are any non-integer dosages between the ranges, for example for the range “about 2 mg to about 10 mg,” values including about 2 mg, about 2.1 mg, about 2.2 mg, about 2.3 mg, about 2.4 mg, about 2.5 mg, about 2.6 mg, etc., are contemplated.

Any dosage of the preceding paragraph may be administered as one, two, three, or four doses during a single day. For example, the 2 mg dose may be administered as a 1 mg dose administration followed by a second 1 mg dose administration after an elapsed period of time, to arrive at the 2 mg dose. For further example, the 2 mg dose can be administered during a single day by administering 0.5 mg, allowing time to elapse, administering a second 0.5 mg, allowing time to elapse, administering a 3^{rd} 0.5 mg, and allowing time to elapse and administering a 4^{th} 0.5 mg to result in 2 mg total administered throughout the day.

Thus, in some embodiments, administration of 2 mg of Compound 1 comprises administration of 1 mg, allowing a defined period of time to elapse and administering a 2^{nd} dose of 1 mg. In some embodiments, administration of 5 mg of Compound 1 comprises administration of 2.5 mg, allowing a defined period of time to elapse and administering a 2^{nd} dose of 2.5 mg. In some embodiments, administration of 10 mg of Compound 1 comprises administration of 5 mg, allowing a defined period of time to elapse and administering a 2^{nd} dose of 5 mg. In some embodiments, administration of 15 mg of Compound 1 comprises administration of 7.5 mg, allowing a defined period of time to elapse and administering a 2^{nd} dose of 7.5 mg. In some embodiments, administration of 30 mg of Compound 1 comprises administration of 15 mg, allowing a defined period of time to elapse and administering a 2^{nd} dose of 15 mg. The defined period of time may be determined by the clinician and subject to individual patient metabolic considerations. In some embodiments, the defined period of time is between about 2.5 hr to about 5 hr, about 5 hr to about 7.5 hr, about 7.5 hr to about 10 hr, about 10 hr to about 12.5 hr, about 12.5 hr to about 15 hr, about 15 hr to about 17.5 hr, about 17.5 hr to about 20 hr, about 20 hr to about 22.5 hr, or about 22.5 hr to about 24 hr.

In some embodiments, a TYK2 inhibitor (e.g., Compound 1) or a pharmaceutically acceptable salt thereof is administered (e.g., orally) at a dose of up to 2 mg to the patient. In some embodiments, a TYK2 inhibitor (e.g., Compound 1) or a pharmaceutically acceptable salt thereof is administered (e.g., orally) at a dose of up to 5 mg to the patient. In some embodiments, a TYK2 inhibitor (e.g., Compound 1) or a pharmaceutically acceptable salt thereof is administered (e.g., orally) at a dose of up to 10 mg to the patient. In some embodiments, a TYK2 inhibitor (e.g., Compound 1) or a pharmaceutically acceptable salt thereof is administered (e.g., orally) at a dose of up to 15 mg to the patient. In some embodiments, a TYK2 inhibitor (e.g., Compound 1) or a pharmaceutically acceptable salt thereof is administered (e.g., orally) at a dose of up to 20 mg to the patient. In some embodiments, a TYK2 inhibitor (e.g., Compound 1) or a pharmaceutically acceptable salt thereof is administered (e.g., orally) at a dose of up to 25 mg to the patient. In some embodiments, a TYK2 inhibitor (e.g., Compound 1) or a pharmaceutically acceptable salt thereof is administered (e.g., orally) at a dose of up to 30 mg to the patient. In some embodiments, a TYK2 inhibitor (e.g., Compound 1) or a pharmaceutically acceptable salt thereof is administered (e.g., orally) at a dose of up to 35 mg to the patient. In some embodiments, a TYK2 inhibitor (e.g., Compound 1) or a pharmaceutically acceptable salt thereof is administered (e.g., orally) at a dose of up to 40 mg to the patient. In some embodiments, a TYK2 inhibitor (e.g., Compound 1) or a pharmaceutically acceptable salt thereof is administered (e.g., orally) at a dose of up to 45 mg to the patient. In some embodiments, a TYK2 inhibitor (e.g., Compound 1) or a pharmaceutically acceptable salt thereof is administered (e.g., orally) at a dose of up to 50 mg to the patient. In some embodiments, a TYK2 inhibitor (e.g., Compound 1) or a pharmaceutically acceptable salt thereof is administered (e.g., orally) at a dose of from about 5 mg to about 30 mg to the patient, such as about 5 mg, about 10 mg, or about 30 mg. In some embodiments, a TYK2 inhibitor (e.g., Compound 1) or a pharmaceutically acceptable salt thereof is administered (e.g., orally) at a dose of from about 10 mg to about 40 mg to the patient. In some embodiments, a TYK2 inhibitor (e.g., Compound 1) or a pharmaceutically acceptable salt thereof is administered (e.g., orally) at a dose of from about 25 mg to about 50 mg to the patient.

As provided in view of preclinical data described herein, a TYK2 inhibitor (e.g., Compound 1) or a pharmaceutically acceptable salt thereof, or a pharmaceutical composition thereof, is administered to a patient at a dosing schedule appropriate to give the desired disease regression with minimum side effects. In some embodiments, a TYK2 inhibitor (e.g., Compound 1) or pharmaceutical composition thereof is administered to a patient daily (QD) for a period between about 1 day to about 7 days, about 1 week to about 3 weeks, about 3 weeks to about 6 weeks, about 6 weeks to about 9 weeks, about 9 weeks to 12 weeks, about 12 weeks to about 15 weeks, or about 15 weeks to about 18 weeks. In some embodiments, a TYK2 inhibitor (e.g., Compound 1) or pharmaceutical composition thereof is administered to a patient daily (QD) for 2 weeks, 4 weeks, 6 weeks, 9 weeks, 12 weeks, 15 weeks, or 18 weeks.

In some embodiments, a TYK2 inhibitor (e.g., Compound 1) or a pharmaceutically acceptable salt thereof or pharmaceutical composition thereof is administered to a patient at a single daily dose of 2-200 mg for 2 weeks. In some embodiments, a TYK2 inhibitor (e.g., Compound 1) or pharmaceutical composition thereof is administered to a patient at multiple daily dose of 20-200 mg for 2 weeks. In some embodiments, a TYK2 inhibitor (e.g., Compound 1) or pharmaceutical composition thereof is administered to a patient at a daily dose of 2, 5, 10, 15, or 30 mg for 2 weeks, 4 weeks, 6 weeks, 9 weeks, 12 weeks, 15 weeks, or 18 weeks.

### 7. Formulation and Combinations

### Pharmaceutically Acceptable Compositions

The compounds and compositions, according to the method described herein, may be administered using any amount and any route of administration effective for treating or lessening the severity of any disorder disclosed herein. The exact amount required will vary from subject to subject, depending on the species, age, and general condition of the subject, the severity of the infection, the particular agent, its mode of administration, and the like. Compounds described herein are preferably formulated in dosage unit form for ease of administration and uniformity of dosage. The expression “unit dosage form,” as used herein, refers to a physically discrete unit of agent appropriate for the patient to be treated. It will be understood, however, that the total daily usage of the compounds and compositions described herein will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

According to another embodiment, a composition can include a compound described herein or a pharmaceutically acceptable derivative thereof and a pharmaceutically acceptable carrier, adjuvant, or vehicle. The amount of compound in compositions described herein is such that is effective to measurably inhibit a TYK2 protein kinase, or a mutant thereof, in a biological sample or in a patient. In certain embodiments, the amount of compound in compositions described herein is such that is effective to measurably inhibit a TYK2 protein kinase, or a mutant thereof, in a biological sample or in a patient. In certain embodiments, a composition described herein is formulated for administration to a patient in need of such composition. In some embodiments, a composition described herein is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound described herein that, upon administration to a recipient, is capable of providing, either directly or indirectly, a compound described herein or an inhibitory active metabolite or residue thereof.

As used herein, the term “inhibitory active metabolite or residue thereof” means that a metabolite or residue thereof is also an inhibitor of a TYK2 protein kinase, or a mutant thereof.

Compositions described herein may be administered orally, parenterally, by inhalation spray, topically, rectally, intracisternally, intraperitoneally, nasally, buccally, vaginally or via an implanted reservoir. In certain embodiments, the compounds described herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, perfuming, flavoring or coloring agents may also be added.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Other topical dosage forms include ointments, pastes, creams, lotions, gels, powders, solutions, sprays, inhalants or patches. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. The active component is admixed under sterile conditions with a pharmaceutically acceptable carrier and any needed preservatives or buffers as may be required. Absorption enhancers can also be used to increase the flux of the compound across the skin. The rate can be controlled by either providing a rate controlling membrane or by dispersing the compound in a polymer matrix or gel (e.g. in a transdermal patch applied topically).

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agcnts.

Most preferably, pharmaceutically acceptable compositions described herein are formulated for oral administration. Such formulations may be administered with or without food. In some embodiments, pharmaceutically acceptable compositions described herein are administered without food. In other embodiments, pharmaceutically acceptable compositions described herein are administered with food.

The amount of compounds described herein that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

Liquid dosage forms for oral administration may be used and include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

Depending upon the particular condition, or disease, to be treated, additional therapeutic agents, which are normally administered to treat that condition, may be administered in combination with compounds and compositions described herein. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations described herein may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies described herein, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination described herein may be administered with another therapeutic agent simultaneously or sequentially in separate unit dosage forms or together in a single unit dosage form.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, a composition can include a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, a method of treating a psoriasis and/or psoriatic arthritis can include administering to a patient in need thereof a compound of formula I (e.g., Compound 1) and one or more additional therapeutic agents. Such additional therapeutic agents may be small molecules or recombinant biologic agents and include, for example, clobetasol, methotrexate, Humira®, Stelara®, triamcinolone, ustekinumab, adalimumab, Cosentyx®, Remicade®, Taltz®, Skyrizi®, Tremfya®, etanercept, Ilumya®, Avsola®, guselkumab, Inflectra®, ixekizumab, Renflexis®, risankizumab, secukinumab, tildrakizumab, fluocinonide, triamcinolone, Elocon®, calcipotriene, mometasone, Clobex®, Dovonex®, prednisone, desonide, hydrocortisone, Soriatanc®, Taclonex®, Tazorac®, Acitrctin®, Cyclosporinc®, bctamcthasonc, betamethasone/calcipotriene, halobetasol, Temovate®, Kenalog®, Kenalog-40®, Neoral®, Desoximetasone®, Fluocinonide-E®, Otrexup®, Trexall®, coal tar, Enstilar®, fluocinolone, tazarotene, Topicort®, calcitriol, Cortizone-10®, dexamethasone, Kenalog-10®, Locoid®, methylprednisolone, prednisolone, Rasuvo®, RediTrex®, Taclonex Scalp®, tacrolimus, Triderm®, Vanos®, Vectical®, Acthar®, Ala-Cort®, Ala-Scalp®, Ala-Scalp HP®, alclometasone, ammonium lactate/halobetasol, Aquanil HC®, Aristocort A®, Aristocort R®, Aristospan®, Beta HC®, Bioelements Immediate Comfort®, Caldecort®, Cinolar®, Clinacort®, Cloderm®, Cordran®, Cordran Tape®, Cortizone-5®, Dermarest Plus Anti-Itch®, Dermovate®, Dermtex HC®, diflorasone, flurandrenolide, Gengraf®, Halog®, infliximab, Itch-X Lotion®, Locoid Lipocream®, NuCort®, Olux®, Olux-E®, Oxsoralen-Ultra®, Pandel®, Psoriasin®, Sarnol-HC®, Sernivo®, Synalar®, Texacort®, Trianex®, Tritocin®, U-Cort®, Abrilada®, amcinonide, Amjevita®, ammonium lactate/urea, Analpram-HC®, Analpram E®, Anthraforte®, anthralin, Anthrascalp®, Apexicon®, ApexiCon E®, Balnetar®, Betatar Gel®, brodalumab, Bryhali®, Calcitrene®, Capex®, Carb-O-Lac5®, Carb-O-Lac HP®, Clobevate®, clocortolone, Clodan®, coal tar/salicylic acid/sulfur, coal tar/salicylic acid, Cordran SP®, Cormax®, Cormax Scalp®, corticotropin, Cutar®, Cyclocort®, Cyltezo®, Derma-Smoothe/FS®, Derma-Smoothe®/FS (Body Oil), Derma-Smoothe®/FS (Scalp), Dermatop®, Desonate®, DesOwen®, DHS Tar Shampoo®, Doak Tar®, Dritho-Scalp®, Drithocreme®, Duobrii®, Elta Tar®, Embeline®, Embeline E®, Epifoam®, Estar®, Fototar®, H.P. Acthar Gel®, Hadlima®, halcinonide, halobetasol/tazarotene, Halonate®, HC Pram®, Hulio®, hydrocortisone/pramoxine, hydroxyurea, Hyrimoz®, Impoyz®, Ionil T®, Ixifi®, Kalosar®, Kerasal Ultra 20®, LoKara®, Medotar®, methoxsalen, MG217 Medicated Tar®, Neutrogena T®/Derm, Neutrogena T®/Gel, Nolix®, Novacort®, Oxipor VHC®, Pramosone®, prednicarbate, Proctofoam HC®. RA Acne®, Resinol®, resorcinol, Scytera®, Siliq®, Sorilux®, Synalar Ointment®, Tarsum®, Theraplex T®, Tovet®, Ultralytic®, Ultralytic 2®, Ultravate®, Vendeso®, Wynzora®, Yusimry®, Zithranol®, and Zithranol-RR®.

In another embodiment, a method of treating an inflammatory disease, disorder or condition can include administering to a patient in need thereof a compound of formula I and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), cmtricitabinc (Emtriva®), lamivudinc (Epivir®), lamivudinc/zidovudinc (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, a method of treating rheumatoid arthritis can include administering to a patient in need thereof a compound of formula I and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, a method of treating osteoarthritis can include administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodics such as tanczumab.

In some embodiments, a method of treating cutaneous lupus erythematosus or systemic lupus erythematosus can include administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, a method of treating Crohn's disease, ulcerative colitis, or inflammatory bowel disease can include administering to a patient in need thereof a compound of formula I and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, a method of treating asthma can include administering to a patient in need thereof a compound of formula I and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, a method of treating COPD can include administering to a patient in need thereof a compound of formula I and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salneterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, a method of treating a hematological malignancy can include administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, a method of treating a solid tumor can include administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, a method of treating a hematological malignancy can include administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, a method of treating diffuse large B-cell lymphoma (DLBCL) can include administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, a method of treating multiple myeloma can include administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, a method of treating or lessening the severity of a disease can include administering to a patient in need thereof a compound of formula I and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholccystitis, chronic graft rcjcction, colitis, conjunctivitis, Crohn's discasc, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, a method of treating or lessening the severity of a disease can include administering to a patient in need thereof a compound of formula I and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, a method of treating or lessening the severity of a disease can include administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vemal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine ophthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments a method of treating or lessening the severity of a disease can include administering to a patient in need thereof a compound of formula I and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, a method of treating or lessening the severity of a disease, can include administering to a patient in need thereof a TYK2 pseudokinase (JH2) domain binding compound and a TYK2 kinase (JH1) domain binding compound. In some embodiments, the disease is an autoimmune disorder, an inflammatory disorder, a proliferative disorder, an endocrine disorder, a neurological disorder, or a disorder associated with transplantation. In some embodiments the JH2 binding compound is a compound of formula I. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

According to one embodiment, a method of inhibiting protein kinase activity in a biological sample can include the step of contacting said biological sample with a compound described herein, or a composition comprising said compound.

According to another embodiment, a method of inhibiting TYK2, or a mutant thereof, activity in a biological sample can include the step of contacting said biological sample with a compound described herein, or a composition comprising said compound. In certain embodiments, a method of irreversibly inhibiting TYK2, or a mutant thereof, activity in a biological sample can include the step of contacting said biological sample with a compound described herein, or a composition comprising said compound.

In another embodiment, a method of selectively inhibiting TYK2 over one or more of JAK1, JAK2, and JAK3 is described. In some embodiments, a compound described herein is more than 2-fold selective over JAK1/2/3. In some embodiments, a compound described herein is more than 5-fold selective over JAK1/2/3. In some embodiments, a compound described herein is more than 10-fold selective over JAK1/2/3. In some embodiments, a compound described herein is more than 50-fold selective over JAK1/2/3. In some embodiments, a compound described herein is more than 100-fold selective over JAK1/2/3.

The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of TYK2 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment a method of inhibiting protein kinase activity in a patient can include the step of administering to said patient a compound described herein, or a composition comprising said compound.

According to another embodiment, a method of inhibiting activity of TYK2, or a mutant thereof, in a patient can include the step of administering to said patient a compound described herein, or a composition comprising said compound. According to certain embodiments, a method of reversibly or irreversibly inhibiting one or more of TYK2, or a mutant thereof, activity in a patient can include the step of administering to said patient a compound described herein, or a composition comprising said compound. In other embodiments, a method for treating a disorder mediated by TYK2, or a mutant thereof, in a patient in need thereof, can include the step of administering to said patient a compound described herein or pharmaceutically acceptable composition thereof. Such disorders are described in detail herein.

Depending upon the particular condition, or disease, to be treated, additional therapeutic agents that are normally administered to treat that condition, may also be present in the compositions described herein. As used herein, additional therapeutic agents that are normally administered to treat a particular disease, or condition, are known as “appropriate for the disease, or condition, being treated.”

A compound described herein may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal*); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination described herein can include a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination described herein can include a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed, under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; isis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GleevecT) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-([(2,5-dihydroxyphenyl)methyl]amino)-benzoic acid adamantyl ester; NSC 680410, adaphostin); 1) compounds targeting, decreasing or inhibiting the activity of the epidermal growth factor family of receptor tyrosine kinases (EGFRi ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PT3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in the methods described herein include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds described herein can be found in WO2008039218 and WO2011090760, the entirety of which are incorporated herein by reference.

Further examples of SYK inhibitory compounds, and conditions treatable by such compounds in combination with compounds described herein can be found in WO2003063794, WO2005007623, and WO2006078846, the entirety of which are incorporated herein by reference.

Further examples of PI3K inhibitory compounds, and conditions treatable by such compounds in combination with compounds described herein can be found in WO2004019973, WO2004089925, WO2007016176, U.S. Pat. No. 8,138,347, WO2002088112, WO2007084786, WO2007129161, WO2006122806, WO2005113554, and WO2007044729 the cntircty of which are incorporated herein by reference.

Further examples of JAK inhibitory compounds, and conditions treatable by such compounds in combination with compounds described herein can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds described herein include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamunc®), cycrolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VelcadeM) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds described herein can be used in combination with standard leukemia therapies, especially in combination with therapies used for the treatment of AML. In particular, compounds described herein can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, a method of treating AML associated with an ITD and/or D835Y mutation, can include administering a compound described herein together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 igGi antibody, Angiozyme (RPI 4610) and Bcvacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds described herein are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound described herein may be mixed with the other drug substance in a fixed pharmaceutical composition or it may be administered separately, before, simultaneously with or after the other drug substance. Accordingly a combination of a compound described herein as hereinbefore described with an anti-inflammatory, bronchodilatory, antihistamine or anti-tussive drug substance, said compound described herein and said drug substance can be in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Baycr), SCH-351591 (Schering- Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds described herein with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents Intemational (e.g. IMS World Publications).

A compound described herein may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound described herein can be administered alone or in combination with one or more other therapeutic compounds, possible combination therapy taking the form of fixed combinations or the administration of a compound described herein and one or more other therapeutic compounds being staggered or given independently of one another, or the combined administration of fixed combinations and one or more other therapeutic compounds. A compound described herein can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents. For example, a compound described herein may be administered with another therapeutic agent simultaneously or sequentially in separate unit dosage forms or together in a single unit dosage form. Accordingly, a single unit dosage form can include a compound described herein, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions described herein should be formulated so that a dosage of between 0.01-100 mg/kg body weight/day of an inventive compound can be administered.

In those compositions which comprise an additional therapeutic agent, that additional therapeutic agent and the compound described herein may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds described herein, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound described herein are another embodiment.

### EXEMPLIFICATION

As depicted in the Examples below, in certain exemplary embodiments, compounds are prepared according to the following general procedures. It will be appreciated that, although the general methods depict the synthesis of certain compounds described herein, the following general methods, and other methods known to one of ordinary skill in the art, can be applied to all compounds and subclasses and species of each of these compounds, as described herein. Additional compounds described herein may be prepared by methods substantially similar to those described herein in the Examples and methods known to one skilled in the art. The TYK2 inhibitors (e.g., Compound 1) described herein can be prepared by methods known to one of ordinary skill in the art, for example, as described in U.S. Pat. No. 11,046,698, the contents of which are incorporated herein by reference in their entireties.

### Example 1: Safety and Tolerability in Healthy Subjects (Study 101)

Study 101 was a randomized, single-center, double-blind, placebo-controlled, single, and multiple ascending dose study in healthy subjects 18 to 55 years of age. This study investigated single ascending doses (SAD) of 5 mg, 20 mg, 75 mg, 100 mg, and 200 mg, and multiple ascending doses (MAD) of 20 mg and 35 mg given QD for 14 days. An additional open-label cohort was also included to evaluate the comparative bioavailability of two drug product formulations, the original d-α-tocopherol polyethylene glycol 1000 succinate (TPGS) formulation and a new spray dried dispersion (SDD) formulation, to assess the PK of single oral doses of the SDD formulation of Compound 1 in healthy subjects under fed and fasted conditions. The SDD formulation is being used in the clinical studies of Compound 1.

In this study, 47 participants (healthy volunteers) were enrolled in single-dose cohorts (35 treated with Compound 1 ranging from 5 mg to 200 mg, and 12 treated with placebo). Treatment with Compound 1 was generally safe and well tolerated. A total of 14 of 35 (40%) participants treated with Compound 1 experienced at least one treatment-emergent adverse event (TEAE), compared with 3 of 12 (25%) treated with placebo. The most common AEs associated with Compound 1 treatment were acneiform dermatitis/papular rash and aphthous stomatitis; these events were mild and did not lead to treatment discontinuation. There were no deaths, serious or severe AEs, or AEs leading to discontinuation from study or study drug in either active- or placebo-treated participants. No adverse trends were noted in safety laboratory tests, ECGs, vital signs, or physical exam findings.

In this study, 16 participants (healthy volunteers) were enrolled in multiple-dose cohorts (12 treated with either 20 mg or 35 mg of Compound 1 daily for two weeks and 4 treated with placebo daily for two weeks). Treatment with Compound 1 was generally safe and well tolerated. A total of 12 of 12 (100%) participants treated with Compound 1 had at least one TEAE during the study period, compared with 2 of 4 (50%) who received placebo. The most common (≥2 subjects) TEAEs observed by preferred term were acneiform dermatitis, reported in 8 subjects treated with Compound 1 (all 6 who received 20 mg and in 2 who received 35 mg daily for 2 weeks). Papular rash was reported in 3 subjects treated with 35 mg Compound 1. All events of acneiform dermatitis or papular rash were deemed to be drug related. They were all mild in intensity and resolved within one to two weeks of onset without requiring treatment discontinuation. Aphthous ulcer occurred in 2 subjects in the 20 mg group and 1 in the 35 mg group, all of whom also experienced acnciform dermatitis. All events were considered drug-related. None of these events were observed in the placebo group. There were no deaths, serious or severe AEs, or AEs leading to discontinuation from the study. One participant in the 20 mg group discontinued treatment due to atrial fibrillation associated with hyperthyroidism but completed study follow-up. This event was deemed unrelated to study drug and was not serious. No adverse trends were noted in safety laboratory tests, ECGs, vital signs, or physical exam findings.

Of 6 subjects enrolled in the open-label cohort, only 1 TEAE of oral herpes was observed in 1 subject (16.7%) after receiving a single dose of 35 mg Compound 1 (SDD formulation) under fasting conditions. This TEAE was mild in severity and was judged as unrelated to Compound 1 by the investigator. This event was also a TEAE of special interest and was resolved before the end of study. No SAEs were observed and none of the subjects experienced TEAEs leading to dose discontinuation during the study period.

Pharmacokinetic Results: In the study, the absorption of Compound 1 was generally rapid with the mean peak plasma concentrations observed at a median time to maximum observed concentration (Tmax) of 3-5 hours postdose in the SAD Cohorts 1-6 and the MAD Cohorts 7 and 8. For the SAD Cohort 9, Periods 1 and 2, Compound 1 (as a TPGS formulation and an SDD formulation, respectively) was administered to the fasted subjects and Tmax was similar to the other SAD cohorts and was observed at a median time of 4-5 hours postdose. For the SAD Cohort 9, Period 3, Compound 1 (as an SDD formulation) was administered to the fed subjects and Tmax was observed at a median of 8 hours postdose and individual values ranged from 8-24 hours postdose.

For Cohorts 1-3 (doses of 5, 20, and 75 mg Compound 1), the mean maximum observed concentration (Cmax) increased in an approximately dose-proportional manner.

(21.6-237.4 ng/mL, or an 11-fold increase in Cmax over a 15-fold increase in dose) and inter-subject variability was moderate for Cmax (coefficient of variation [CV] of 20-28%). For doses of 100, 150, and 200 mg (Cohorts 5, 4, and 6 respectively), the mean Cₘₐₓ increased in a less than dose proportional manner (232.5-250 mg/mL) and inter-subject variability was somewhat greater (CV 35-57%).

Similarly, for Cohorts 1-3 (doses of 5, 20, and 75 mg Compound 1), the mean area under the plasma concentration versus time curve from time 0 to infinity (AUC0-inf) increased in an approximately dose-proportional manner (589.4-8369.8 hr*ng/mL, or a 14-fold increase in AUC0-inf over a 15-fold increase in dose). For doses of 100 and 150 mg (Cohorts 5 and 4), the mean AUC0-inf increased in a less than dose proportional manner (10744.9 and 12190.8 hr*ng/mL, respectively). The exposure in Cohort 6 (dose of 200 mg) did not increase further and the mean AUC0-inf was 9015 hr*ng/mL. For the single dose Cohorts 1-6, the percentage of the area under the concentration versus time curve (AUC %) extrapolated was moderate ranging from 9.9-30.9%.

Upon multiple dose administration for 14 days at a 20 or 35 mg daily dose (Cohorts 7 and 8, respectively), the accumulation ratio for Cₘₐₓ was 2.6 and 2.2, respectively and the area under the plasma concentration versus time curve from time 0 to the end of the dosing period (AUC0-tau) was 2.9 and 2.4, respectively. After repeat dosing at 20 or 35 mg, on day 14, the minimum observed concentration (Cₘᵢₙ) at 24 hours prior to the next daily dose was 82.7 and 169.9 ng/mL, respectively.

Across the single dose Cohorts 1-6 and the multiple dose Cohorts 7 and 8, the terminal half-life (t1/2) was consistent across the dose levels tested and ranged from 17.1 to 37.4 hours.

Cohort 9 Periods 1 and 2 allowed the assessment of comparative bioavailability of the TPGS formulation (used to formulate Compound 1 for dosing of Cohorts 1-8) to the SDD formulation at as a single dose of 35 mg to fasting subjects. Overall, exposure was similar after a single dose of Compound 1 as TPGS or SDD formulation as assessed by Cₘₐₓ (112.3 ng/mL and 148 ng/mL, respectively) and AUC_{0-inf} of (3146.3 and 4027.5 hr*ng/mL, with relative oral bioavailability (F %) of the SDD formulation to the TPGS formulation calculated to be 128%). Cohort 9 Periods 2 (fasted, SDD formulation) and 3 (fed, SDD formulation) allowed assessment of the potential for food effect on exposure. Generally, exposure in the two periods was similar with the ratio of fed to fasted pharmacokinetic parameters all close to unity for Cₘₐₓ (0.91), AUC_{0-inf} (1.07), area under the plasma concentration versus time curve from time 0 to the time of last measurable concentration (AUC₀₋ₗₐₛₜ; 1.02), and t1/2 (0.95). Thus, no food effect was observed in this study.

Overall, renal elimination of the parent drug represented a low but measurable percentage of the total administered dose of Compound 1 and the fraction of the dose excreted in urine (FE %) ranged between 0.37-1.89% of dose. Thus, renal excretion may contribute to clearance of the parent drug but is expected to be a relatively minor route of clearance based on this study. Renal clearance (CLr) ranged from 0.06-0.30 L/hr.

Pharmacodynamic Results: The PD effect of treatment within this study was assessed using an ex-vivo immune-assay measuring the amount of TYK2-dependent interferon gamma (IFNγ) produced by whole blood samples that were stimulated with the cytokines IL-12 and IL-18 at baseline (predose) and following treatment. Increasing exposures to Compound 1 led to greater reduction in IFNγ, confirming a robust effect of this compound on biological endpoints relevant to the pathogenesis of several autoimmune diseases.

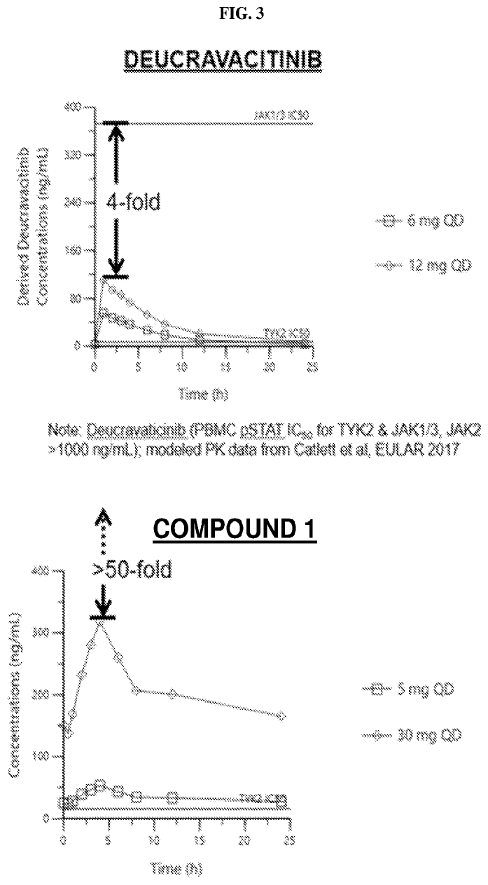

To assess the relative risk of JAK1/3 inhibition for deucravacitinib and Compound 1, we modeled peak concentrations of each compound achieved in human trials against the physiologically relevant IC₅₀ inhibition level. FIG. 3 illustrates the fold selectivity of deucravacitinib and Compound 1 in human cells (peripheral blood monocytes, or PBMCs) for JAK1/3 IC₅₀ inhibition. Based on the published PK data from deucravacitinib's Phase 1a clinical trial, deucravacitinib administered at 12 mg once-daily showed a four-fold margin between the peak concentration achieved (approximately 120 ng/ml) and the IC₅₀ concentration of approximately 370 ng/ml for JAK1/3 pSTAT inhibition. Without wishing to be bound by theory, it is believed that the margin for Compound 1, based on PK data from our Phase 1a clinical trial, is greater than 50-fold based on a peak concentration of approximately 320 ng/ml following administration of 30 mg once-daily. We have not conducted head-to-head comparative preclinical studies or clinical trials of deucravacitinib and Compound 1. As a result, comparative conclusions cannot be drawn between the data presented by BMS on deucravacitinib and the data we have observed for Compound 1.

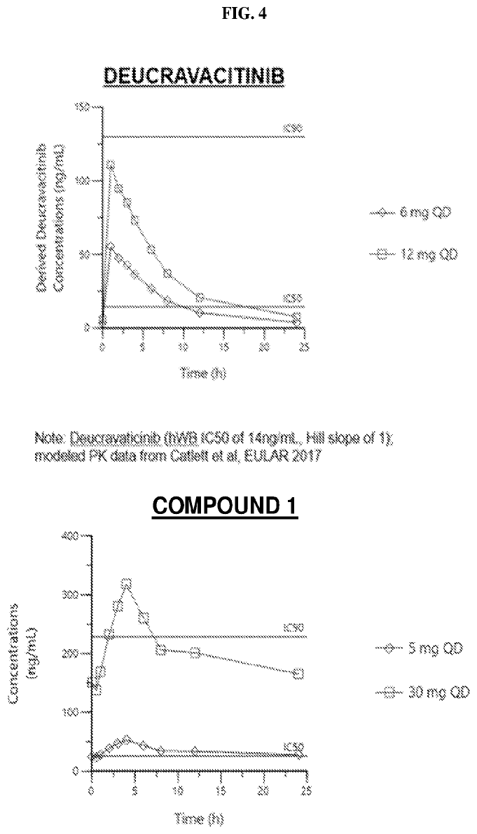

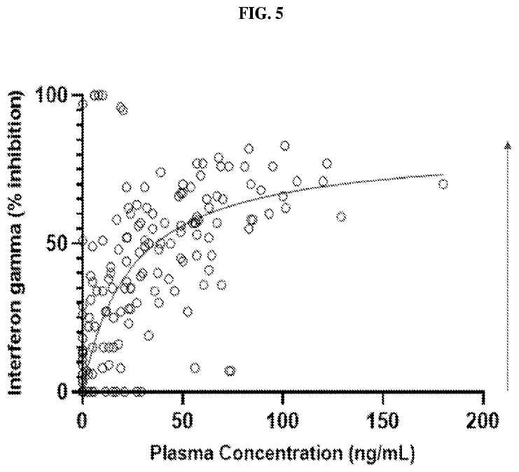

Without wishing to be bound by theory, we believe that increased TYK2 inhibition over 24 hours has the potential to lead to an increased response in patients with inflammatory and autoimmune diseases, in particular those such as ulcerative colitis and Crohn's disease that require a higher level of cytokine modulation. In the Phase 2b trial of deucravacitinib dosed at 6 mg once-daily in moderate-to-severe ulcerative colitis, deucravacitinib did not achieve its primary endpoint of clinical remission at 12 weeks or its secondary endpoints of clinical response using the modified Mayo score, endoscopic response and histological improvement. BMS is currently conducting a second trial of deucravacitinib in ulcerative colitis that includes a higher dose. Based on the PK data observed in our Phase 1a clinical trial shown below, we believe the trialed doses of Compound 1 may achieve greater TYK2 inhibition over a 24 hour-period than the Phase 2b trialed doses of deucravacitinib, which we believe supports further evaluation of Compound 1 in ulcerative colitis. See FIG. 4.

We also modeled IC₅₀ values for TYK2 inhibition in human whole blood cells based on the PK data from the Phase 1a clinical trial for deucravacitinib and similar data from our Phase 1a clinical trial. Based on our modeling as shown in FIG. 4, the 6 mg once-daily dose of deucravacitinib (the dose used in the deucravacitinib Phase 3 psoriasis trials and Phase 2b ulcerative colitis trial) provided TYK2 IC₅₀ coverage in human whole blood for approximately 10 hours. The Compound 1 doses of 5 mg and 30 mg once-daily would be able to maintain TYK2 IC50 coverage for 24 hours based on this model. Furthermore, the 30 mg dose of Compound 1 achieved approximately IC₇₅ coverage for 24 hours and IC₉₀ coverage for several hours per day in this model.

Without wishing to be bound by theory, it is believed that the PK and pharmacodynamic (PD) data from our Phase 1a trial enables upward dosing flexibility for Compound 1 given its wide therapeutic index relative to JAK1/3 inhibition. Based on publicly available information, BMS has not explored doses higher than 12 mg twice-daily in any of its patient studies for any indication. As shown in FIG. 5, our PK and PD data from our Phase 1a trial demonstrated a favorable, linear response between the dose and drug levels in the blood and a robust response, including dose-dependent inhibition of IFNy, a cytokine modulated by TYK2 signaling. The predicted effective dose range, based on the PD data (through concentration greater than IC₅₀ in human white blood cells), was 3 to 30 mg once-daily.

### Example 2: Safety and Tolerability in Psoriatic Subjects (Study 102)

This study was a Phase 1, randomized, multicenter, double-blind, placebo-controlled multiple ascending dose study of Compound 1 in subjects with moderate to severe plaque psoriasis. The study objective was to provide preliminary evidence of safety, tolerability, pharmacokinetics, pharmacodynamics, and early efficacy in a moderate to severe plaque psoriasis population.

The study enrolled a total of 26 subjects, who were randomized to daily treatment with either placebo (N=5) or 1 of 3 doses of Compound 1 (N=21) for a total duration of 28 days. Dose levels were 5 mg (N=8), 10 mg (N=7), or 30 mg (N=6). The TPGS formulation was used for the 5 mg dose level, and the SDD formulation was used for the 10 mg and 30 mg dose levels. Food intake was not restricted except when performing study procedures during clinic visits.

Efficacy Results: Exploratory efficacy data in psoriasis (PASI, static PGA [sPGA]) were obtained in this study. This study had a small sample size (25 subjects contributed to efficacy data) and treatment duration was limited to 28 days. A total of 25 subjects (N=8 for 5 mg, N=7 for 10 mg, N=5 for 30 mg, and N=5 for placebo) were included in the efficacy analysis set, which included subjects who had PASI and/or sPGA data at Day 1 and Day 28. Efficacy data were complete for these 25 subjects, and no imputation was needed for missing data. PGA assessment was performed according to Table 1.

TABLE 1
Assessment of Physician Global Assessment (PGA)

| Score | Grade | Description |
|---|---|---|
| 0 | Clear | No signs of psoriasis; post-inflammatory hyperpigmentation may be present |
| 1 | Almost clear | No thickening; normal to pink coloration; no to minimal focal scaling |
| 2 | Mild | Just detectable to mild thickening; pink to light red |
| | | coloration; predominantly fine scaling |
| 3 | Moderate | Clearly distinguishable to moderate thickening; dull to bright red; |
| | | clearly distinguishable to moderate erythema; moderate scaling |
| 4 | Severe | Severe thickening with hard edges; bright to deep dark red |
| | | coloration; severe/coarse scaling covering almost all or all lesions |

Treatment with Compound 1 showed a dose-dependent trend in reduction of disease severity, with mean percent reduction from PASI score at Day 28 compared to Day 1 of 30% (mean PASI reduced from 15.4 on Day 1 to 10.9 on Day 28), 47% (mean PASI reduced from 18.2 on Day 1 to 9.6 on Day 28), and 48% (mean PASI reduced from 20.0 on Day 1 to 10.4 on Day 28), in the 5, 10, and 30 mg groups, respectively, compared with a 26% reduction (mean PAST reduced from 13.5 on Day 1 to 10.0 on Day 28) in the placebo group.
PASI-50 was achieved in 13% (1/8), 57% (4/7), and 40% (2/5) in the 5, 10, and 30 mg groups, respectively, compared to 0% (0/5) in the placebo group.
PASI-75 was achieved in 1 subject (1/5; 20%) in the 30 mg group but not achieved in the other groups. The same subject also achieved PAST 90.
Treatment with Compound 1 also improved the sPGA score compared to placebo, with one subject in the 30-mg cohort achieving an sPGA of 1 (minimal disease) at Day 28.

Safety results: Treatment with Compound 1 was generally safe and well tolerated. There were no deaths or serious AEs. TEAEs occurred in 38% (3/8), 57% (4/7), and 67% (4/6) of subjects receiving 5, 10, or 30 mg Compound 1, compared with 20% (1/5) of subjects in the placebo group. All TEAEs were mild (Grade 1) or moderate (Grade 2) in intensity with the exception of one severe (Grade 3) adverse event of neutropenia in a subject treated with 30 mg of Compound 1 that led to discontinuation of treatment. This event was observed on Day 8 of treatment resulting in discontinuation of study drug on Day 12. The subject's neutrophil count was normal on Day 15,

3 days after treatment was stopped. The Grade 3 neutropenia was the only adverse event constituting Grade 2 or higher hematologic toxicity observed in this study. It was deemed related to study drug but not serious. There were no events of acneiform dermatitis, papular rash, or aphthous ulcer reported in this study. There were no clinically significant changes in vital signs, physical exam, or ECG. One subject in the 30 mg cohort received only one dose of study drug and discontinued the study after Day 1 due to a positive TB test. This subject was included in the safety analysis set, but not in the efficacy analysis.

Pharmacokinetic results: In this study, absorption of Compound 1 was generally rapid with mean peak plasma concentrations observed at a median of 3-4 hours postdose in Cohorts 1-3 (5 to 30 mg once daily). Between 5 and 30 mg doses, mean Cmax generally increased in a slightly greater than dose-proportional manner under steady-state conditions (i.e., on Day 28 of dosing), with an 8-fold increase in Cmax over a 6-fold increase in dose. Similarly, the observed exposure over the dosing interval, AUC0-tau, increased in a greater than dose-proportional manner with a 11-fold increase in AUC0-tau over a 6-fold increase in dose. Owing to the small sample size, and the large inter-subject variability observed in the study (55-111% CV for Cmax and 47-269% CV for AUC0-tau), this lack of observed dosc-proportionality may not be pharmacokinetically or pharmacologically relevant. The observed accumulation ratio, between Day 1 and Day 28, in this study was consistent with the value obtained in the previous study in healthy volunteers.

Risk: No important identified risks emerged from Study 101 in healthy volunteers or Study 102 in subjects with moderate to severe plaque psoriasis.

In comparison, in clinical trials of Otezla® conducted by a third party, the mean PASI reduction reported in the Phase 3 trial of Otezla® dosed at 30 mg twice-daily in moderate-to-severe psoriasis patients was 37% at four weeks. Mean PASI reductions for deucravacitinib have not been reported and BMS did not conduct a Phase 1b trial with deucravacitinib. In our four-week Phase 1b trial, Compound 1 demonstrated exploratory signals of clinical activity which we believe to be consistent with the four-week results of deucravacitinib and guselkumab (an anti-IL-23 monoclonal antibody) in their Phase 2b trials. In those trials, clinical responses increased substantially from week four to week twelve. In the dose ranges that were used in the Phase 3 trials for deucravacitinib and guselkumab, responses increased from three- to four-fold at 12 weeks compared to four weeks. Accordingly, we believe we may see an increase in clinical response rates for Compound 1 at twelve weeks, as measured by PASI scores, in our ongoing Phase 2b trial in moderate-to-severe psoriasis.

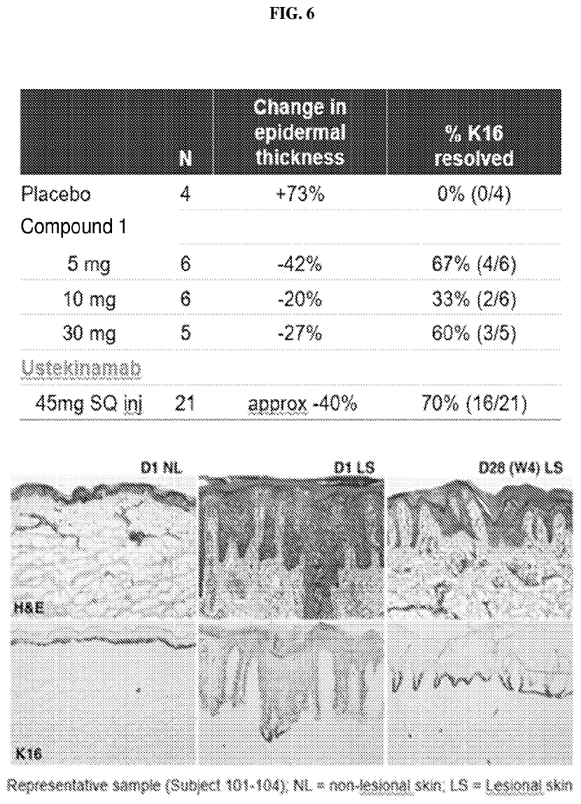

PD Marker Results: We believe PD markers from skin punch biopsies support the exploratory signals of clinical activity for Compound 1 consistent with that expected of an allosteric TYK2 inhibitor. Skin PD markers are evaluated based on the change in epidermal thickness (via hematoxylin and eosin, or H&E staining) and the presence of the type I keratin K16 hyperproliferation marker. K16 expression is upregulated in psoriasis and downregulated when psoriasis improves. As seen in FIG. 6, Compound 1 reduced epidermal thickness and resolved expression of the K16 biomarker in patients across the three dosing cohorts at four weeks (17 patients evaluated).

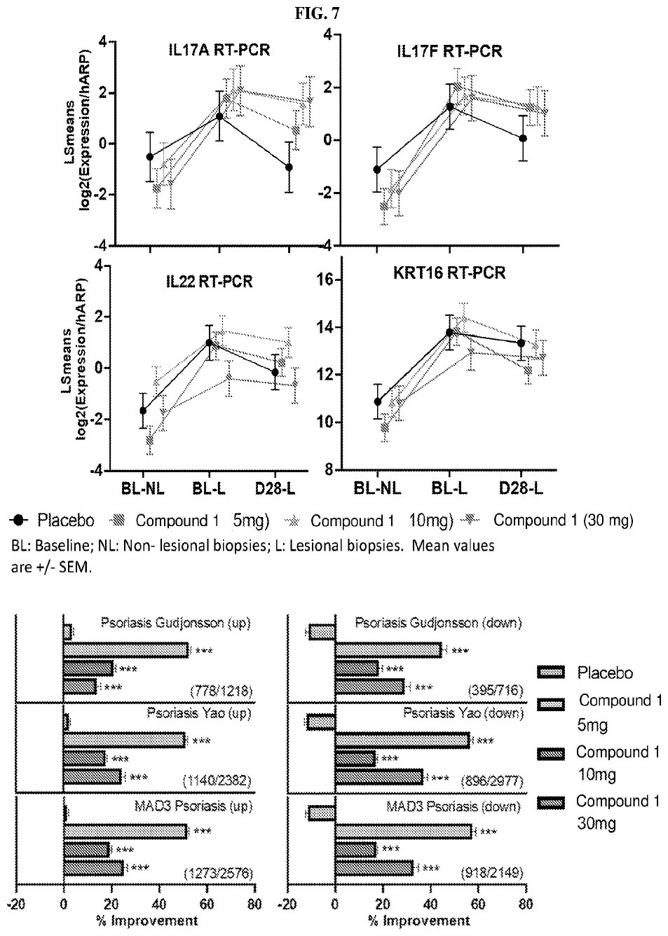

As shown in FIG. 7, treatment for 28 days reduced the RNA expression levels of key cytokines and psoriasis gene signatures in lesional skin. Methods: mRNA from skin biopsies was isolated and assessed for targeted (RT-PCR) or global (microarray) gene expression. For microarray analysis, differential gene expression comparing baseline lesional vs. non-lesional samples (psoriasis transcriptome, PSTR) and comparing baseline lesional vs. Day 28 lesional samples (Compound 1 response) was assessed. The set of genes that were significantly differentially-expressed in lesional vs. non-lesional baseline skin biopsies (PSTR) (FC ≥2, pval <0.05) shows high overlap with several published datasets of differentially-expressed genes in psoriasis lesional vs. non-lesional skin. Results: Quantification of mRNA from skin biopsies showed suggestive decreases in the expression of several psoriasis-related genes by RT-PCR (such as KRT16, IL17A, IL17F, and IL22) and up to 50% improvement in the expression of skin transcriptomes previously associated with psoriasis in lesional skin through microarray analysis (p<0.001 compared to placebo treatment). These results support further development of Compound 1 in psoriasis. Genes that show up or down-regulation in published psoriasis skin datasets show significant improvement following 28-days of treatment with Compound 1 at all doses. Numbers in parentheses are (probeset overlap of Nimbus PSTR and indicated dataset/Total # of probesets in dataset). Percent improvement is assessed as the mean % change (+/−SEM) in expression of the overlapping probesets toward baseline non-lesional expression (*** p<0.001 comparing treatment vs. placebo).

### Example 3: A Phase 1, Randomized, Double-Blind, Placebo-Controlled, Multiple Dose Study of Compound 1 in Healthy Volunteers (Study 104)

### Synopsis

Rationale: In a previous study (Study 101), 2 cohorts of healthy participants received Compound 1 at the dose of 20 mg or 35 mg daily for 2 weeks. Treatment was generally safe and well tolerated, without serious or severe adverse events reported. The present study was designed to continue dose escalation in healthy participants in order to define a relatively broad dose range to help select doses for future studies in patients with psoriasis and other autoimmune diseases. The nonclinical pharmacology, toxicology, and pharmacokinetic (PK) studies and earlier clinical data support the proposed multiple dose study of Compound 1 in healthy participants.

### Objectives and Endpoints

| Objectives | Endpoints |
|---|---|
| Primary | |
| Assess the safety and tolerability of multiple | Frequency and severity of adverse events |
| oral doses of Compound 1 in healthy | |
| participants | |
| Secondary | |
| Assess the PK of multiple oral doses of | AUC₀₋ₜₐᵤ, Cₘₐₓ, tₘₐₓ, and AUC₀₋ₗₐₛₜ. |
| Compound 1 in healthy participants | Minimal observed concentration within |
| | the dosing interval. |
| | Area under the concentration-time curve from |
| | time zero to the end of the dosing interval |
| | AUC₀₋ₜₐᵤ (tau = 24 hours). |
| | % AUCₑₓₜᵣₐₚₜ, t½z, CL/F at steady state, |
| | and Vz/F at steady state. |
| | Accumulation index. |
| Characterize the pharmacodynamics (PD) of | Production of interferon-gamma (IFNγ) from |
| multiple oral doses of Compound 1 in healthy | whole blood treated ex-vivo with IL-12 |
| participants. | following compound administration |

### Overall Design

This was a phase 1, randomized, double-blind, placebo-controlled, multiple dose study of Compound 1 in healthy participants.

The study population was comprised of healthy male participants and/or female participants of non-childbearing potential 18 to 65 years of age. There were two multiple dose cohorts (Cohorts 1 and 2) of 8 healthy participants who received daily doses of Compound 1 (N=6) or placebo (N=2) for 2 weeks. Study participants were admitted to the CRU on Day −1. Participants remained confined in the CRU through completion of all scheduled procedures until Day 22, and then discharged if medically appropriate. Safety labs, PK blood draws, PD blood draws, and adverse event evaluations took place as per the Schedule of Activities (SoA).

Cohort 2 was not enrolled until adequate safety and tolerability from Cohort 1 was demonstrated as determined by the PI and Sponsor.

Patients were questioned about potential COVID-19 exposure/symptoms and tested for COVID-19 via PCR test at the screening visit and at Day −1, and samples were sent to a central laboratory. Patients with a positive PCR test at screening or Day −1 were considered Screen Failures. Participants who develop symptoms consistent with COVID-19 infection after dosing were tested via PCR test and were withdrawn from the study if they had a positive result. This was recorded as an Adverse Event.
Disclosure Statement: This is a sequential group interventional study with 2 cohorts that are Sponsor, participant, and investigator blinded.

### Number of Participants:

There were approximately 16 participants randomly assigned to study intervention and 16 evaluable participants (8 per cohort).

### Intervention Groups and Duration:

16 participants are expected to receive either active study drug or placebo at the doses specified below.

TABLE 1
Cohorts and Dose Levels

| Cohort | Dose Level | Dose |
|---|---|---|
| Cohort 1 | DL1 | 50 mg or matching placebo |
| Cohort 2 | DL2 | 100 mg or matching placebo |

The screening period was approximately 28 days. Participants were confined at the CRU from Day −1 to Day 22. Treatment was given daily for 14 days, and a SFU visit was scheduled to take place on Day 22 prior to discharge from the CRU. The total duration of study participation was approximately 50 days.

### Dose Escalation Stopping Rules

A decision to proceed to the next higher dose from Cohort 1 to Cohort 2 was made jointly by the Sponsor and the P1 following the review of all pertinent blinded safety/tolerability (e.g., AEs, clinical laboratory tests, physical examinations, vital signs, and safety ECGs) and PK data through Day 15 for at least 6 participants in Cohort 1. Together the Sponsor and PI determined whether to continue with the study as planned, continue the study with additional safety evaluations, continue the study with a dose different from that planned for Cohort 2, or discontinue dose escalation.

Dose escalation was terminated if at least 2 participants in a cohort meet any of the following criteria attributable to study drug:
Have a drug-related SAE.
Experience a drug-related grade 3 or higher toxicity.

Dose escalation was terminated if even 1 participant at a given dose level meets the following criterion attributable to study drug:

### 1. Has Evidence of Drug-Induced Liver Injury (DILI).

If treatment-related SAEs, grade ≥3 AEs, and/or evidence to suggest DILI are observed, a PK sample was collected, and the Sponsor notified. At the discretion of the PI, the corresponding participant's treatment assignment may be unblinded to determine whether the events are related to Compound 1 and whether the stopping criteria have been met. Any unblinding of participant treatment was documented.

When applicable, a written statement fully documenting the reasons for study termination was provided to the IRB. The study was monitored by the study team.

### Justification for Dose

Compound 1 has previously been investigated in a first in human study (Study 101) of healthy volunteers in a Single and at two Multiple Dose (SAD and MD) levels. Single doses between 5 mg and 200 mg and multiple doses of 20 mg or 35 mg daily for 2 weeks were generally safe and well tolerated. There were no serious or severe AEs, nor AEs that led to discontinuation of treatment. The most common adverse effect was a form of skin rash, acneiform dermatitis, seen in 7 of 17 (41%) of subjects receiving single doses of 100 mg or higher and 8 of 12 (67%) of subjects receiving multiple doses. While common, these events were all mild in intensity and, even in the multiple dose groups, resolved within 1 to 2 weeks with limited or no intervention and without requiring discontinuation of treatment.

In the single dose cohorts, which used the TPGS formulation, overall exposure to Compound 1 increased in an approximately dose proportional manner between 5 and 75 mg, and in a less than dose proportional manner between 100 and 200 mg. Overall, exposure was similar after a single dose of Compound 1 as TPGS or SDD formulation. Similar oral exposure was observed when Compound 1 with SDD formulation was administered in a fed or fasted state.

In the multiple dose cohorts, which used the TPGS formulation, moderate accumulation of Compound 1 was observed at the 20 and 35 mg daily dose levels, with accumulation ratios of <3 across both Cₘₐₓ and AUC₀₋ₜₐᵤ for both dose regimens. At steady state, treatment with 20 mg achieved a Cₘₐₓ of approximately 207 ng/mL and an AUCₗₐₛₜ of approximately 3160 hr*ng/mL. The corresponding Cₘₐₓ and AUCₗₐₛₜ values observed at steady state with the 35 mg dose given daily for 2 weeks were approximately 325 ng/mL and 5839 hr*ng/mL. The AUC exposure levels achieved with the higher dose tested, 35 mg, were approximately 8- to 9-fold lower than the NOAELs determined in 28-day repeat-dose toxicity studies in monkeys and rats, respectively.

The present study investigated 2 additional multiple doses, 50 mg in Cohort 1 and up to 100 mg in Cohort 2, in order to expand the range of doses studied. The SDD formulation was used in this study. The projected exposure levels following administration of 50 mg and 100 mg doses given daily for 2 weeks are projected to be approximately 7- and 8-fold lower (50 mg) and 3- and 4-fold lower (100 mg) than the NOAELs determined in 28-day repeat-dose toxicity studies in monkeys and rats, respectively.

The nonclinical pharmacology, toxicology, and pharmacokinetic (PK) studies, early clinical data, as well as the modeling data for the 50 mg and 100 mg dose, support the proposed multiple dose study of Compound 1 in healthy participants, with 50 mg as the first dose being tested.

### Check-In Procedures (Day −1)

On the morning of Day −1, all participants returned to the CRU. Inclusion and exclusion criteria were reviewed to ensure participants continue to meet all entry criteria. A second PCR COVID-19 test were also performed, and samples sent to a central laboratory. After the PCR test has been performed, participants remained in an isolated area until the test results are received later that day. Participants who do not meet all the inclusion criteria or meet any of the exclusion criteria on Day −1 (including a positive COVID-19 test) were considered Screen Failures.

Alternates who meet entry criteria at screening also returned to the CRU to complete Day −1 activities. If the alternates were not randomized because the target number of participants per cohort had been met, they were still eligible for the following cohort. Day −1 labs did not need to be repeated if the alternates are enrolled in the study the day following their initial Day −1 assessment.

### Treatment and Monitoring Period

On the morning of Day 1, pre-dose evaluations were obtained. Upon completing review of all criteria, the participant was randomized.

Participants received a single oral dose of Compound 1 or placebo in a blinded manner on the morning of Day 1 and then daily for a total of 14 days. Safety and tolerability were assessed during the treatment period through monitoring, including of vital signs, clinical laboratory tests, 12-lead ECGs, and AEs. Blood samples for PK and PD assessments and urine samples for metabolite profiling were collected at the timepoints as listed. Cardiodynamic monitoring was also conducted.

### SFU Visit/Early Termination

The SFU visit was performed on Day 22 prior to discharge.

Safety and tolerability were assessed through monitoring, including of vital signs, clinical laboratory tests, 12-lead ECGs, and AEs.

In the event of early termination, the procedures listed in the SoA for the SFU visit should be performed. However, a blood sample for PK assessments were also collected. Following the early termination visit, the participant should return to the site for a SFU visit no more than 7 days after the last study drug administration.

### Meal Schedule

Meals and/or snacks were provided as appropriate on Day −1.

Participants were required to fast for a minimum of 8 hours overnight prior to study drug administration and continue to fast for at least 4 hours thereafter on Day 1 only.

Water (except water provided with dosing) was restricted from 1 hour prior to until 1 hour after dosing but was allowed ad libitum at all other times. Other fluids were permitted as part of the standard meals and/or snacks but were restricted at all other times throughout the confinement period.

### Duration of Confinement

Participants were confined to the CRU for 22 days.

### Inclusion Criteria

Participants were eligible to be included in the study only if all the following criteria apply:

### Age

1. Participant must be 18 to 65 years of age inclusive, at the time of signing the informed consent.

### Type of Participant and Disease Characteristics

2. Participants who are medically healthy volunteers with no clinically significant medical history, physical examination, laboratory profiles, vital signs, or ECGs at screening and Day −1 as deemed by the PI. All laboratories should generally be within the normal range specified by the CRU laboratory.
3. Non-smoker (defined as an individual who has not used nicotine-containing products, including cigarettes and e-cigarettes, for at least 3 months prior to dosing).

### Weight

4. Body mass index (BMI) within the range 18-35 kg/m² (inclusive) at screening and clinic admission.

### Sex

5. Males, or females not of childbearing potential. Male participants and their female partners must use two methods of contraception, one considered highly effective (failure rate of less than 1% per year) as defined by CTFG guidelines (CTFG 2014) and the other a barrier method when engaging in sexual intercourse during the study. After final dose administration, male participants and their partners should continue to use contraception and refrain from donating sperm for 90 days. Female participants must have been either surgically sterilized at least 6 months prior to screening or be postmenopausal (FSH test to confirm).

### Informed Consent

6. Capable of giving signed informed consent which includes compliance with the requirements and restrictions listed in the informed consent form (ICF) and in this protocol.

### Exclusion Criteria

Participants were excluded from the study if any of the following criteria apply:

### Medical Conditions

1. Any acute or chronic medical condition, including the presence of laboratory abnormalitics (greater than Grade 1) or electrocardiogram (ECG) examination abnormalitics, or psychiatric illness, that would prevent the participant from signing the Informed Consent form, place the participant at an unacceptable risk if he/she were to participate in the study, or confound the ability to interpret data from the study. Participants with evidence of mild active infection (e.g., upper respiratory, urinary, gastrointestinal) at the time of screening can be brought back for rescreening after symptoms have completely resolved and the appropriate course of treatment has been completed.
2. Female participants of childbearing potential.
3. Positive serology for hepatitis B, hepatitis C, or human immunodeficiency virus (HBsAg, HCV Ab, or HIV Ab positive).
4. Positive PCR test for COVID-19 at the screening or Day −1 visit; or suspected COVID-19 infection within 10 days of the screening visit; or prior contact with another person diagnosed or under investigation for COVID-19 within 10 days of screening. However, subjects who have tested positive for COVID-19 during screening for Cohort 1 may be rescreened for Cohort 2 after they have completed an appropriate quarantine period per CDC guidelines. Subjects with prior COVID-19 infection (based on clinical symptoms or laboratory testing) who have symptomatically recovered, test negative for COVID-19 antigen by PCR and have no sequelae may also be screened for inclusion in the study as deemed appropriate by the PI.
5. Participants with any surgical or medical conditions that may affect the study drug absorption, distribution, metabolism, or excretion.
6. Blood pressure is less than 90/40 mmHg or greater than 140/90 mmHg at screening.
7. Heart rate is lower than 40 bpm or higher than 99 bpm at screening.
8. QTcF (Fridericia correction) interval >450 msec for males or >470 msec for females at screening, or history of prolonged QT syndrome.
9. Blood (including whole blood, platelet, or plasma) donation or significant blood loss within 56 days prior to dosing or planned donation within 30 days of final study medication administration.
10. Inability to tolerate oral medication.
11. History of solid or hematologic malignancy (including pre-malignant states such as myclodysplastic syndrome or lymphoproliferative disorders) within the past 5 years; excludes past history of localized basal cell or squamous cell carcinoma of the skin that has been surgically removed with no evidence of recurrence, ductal carcinoma in situ of the breast successfully treated, and cervical cancer in situ successfully treated.

### Prior/Concomitant Drugs, Supplements, or Procedures

12. Participants who have received any vaccine, including COVID-19, within 28 days of dosing.
13. Participants who have used systemic prescription medications within 30 days (or 5 half-lives of the concomitant therapy, whichever is the longer) of dosing, or who have used over-the-counter, herbal remedies, vitamin supplements, or topical medications within 14 days of dosing. Self-limited use of medication (e.g., Tylenol) can be considered for an exception, if approved by the PI and Sponsor.
14. Participants who plan to have elective medical procedures during the conduct of the study.
15. Participants who engage in recreational drug use or who test positive on drug screen test at screening.
16. Participants who test positive for cotinine.

### Prior/Concurrent Clinical Study Experience

17. Participants who have been administered an investigational drug or an approved drug in an investigational setting within 30 days or 5 half-lives (whichever is longer) or within 6 months of study medication administration if investigational drug is a biologic (e.g., antibody) or who are currently enrolled in an investigational study.

### Diagnostic Assessments

18. Participants who may have any other criteria (such as clinically significant screening blood test result), which in the opinion of the PI, could interfere with the study conduct or outcome.

### Other Exclusions

19. Participants with any known hypersensitivity to any of the excipients contained in the study drug or placebo formulation.
20. Alcohol consumption within 14 days of randomization.

### Lifestyle Considerations

Limitations on diet, substance use, and activity level were required during participation in the study as outlined in the following sections.

### Meals and Dietary Restrictions

Participants are prohibited from consumption of grapefruit or grapefruit juice 14 days before dosing, during all treatment periods, and throughout the period of PK sample collection.

### Caffeine, Alcohol, Tobacco, and Cannabis Restrictions

Consumption of foods and beverages containing the following substances were prohibited as indicated:

Participants may not consume products containing xanthines or caffeine from 48 hours before dosing and throughout the period of PK sample collection.

Participants may not consume alcohol or alcohol containing products within 14 days of dosing or as described for concomitant medication use. Cold medications and other agents containing small amounts of alcohol are allowed if deemed necessary by the PI.

Participants may not use tobacco or nicotine products (including smokeless tobacco, nicotine patches, or nicotine gum) while at the clinical unit and should refrain from use of such products use during the study screening period.

Participants may not use recreational drugs, including Cannabis products (in any form, including oral or sublingual) while at the clinical unit. Participants should also refrain from use of recreational drugs during the study screening period.

### Activity

Participants should limit physical activity to levels sufficient to carry out activities of daily living (e.g., eating, bathing, dressing) from 48 hours prior to dosing through the SFU visit. During this time, no moderate or strenuous activity is allowed (e.g., doubles tennis, 3-5 k runs, marathon training/running, CrossFit training, or weight-lifting).

### Study Intervention(s) Administered

| ARM Name | Cohort 1 | Cohort 2 |
|---|---|---|
| Intervention Name | Compound 1 SDD Drug Product or | Compound 1 SDD Drug Product or |
| | Placebo | Placebo |
| Type | Drug | Drug |
| Dose Formulation | capsule | capsule |
| Unit Dose | 50 mg | 25 mg |
| Strength(s) | | |
| Dosage Level(s) | 50 mg QD for 2 weeks | 100 mg QD for 2 weeks |
| Route of | oral | oral |
| Administration | | |
| Use | Experimental | Experimental |
| IMP and NIMP | IMP | IMP |
| Sourcing | Provided centrally by the Sponsor | Provided centrally by the Sponsor |
| | designee | designee |
| Packaging and | Study Intervention was provided in a | Study Intervention was provided in a |
| Labeling | container. Each container was labeled as | container. Each container was labeled as |
| | required per country requirement. | required per country requirement. |

| | |
|---|---|
| Excipients | Hydroxypropylmethylcellulose-acetate-succinate, Microcrystalline Cellulose, |
| | Mannitol, Croscarmellose Sodium, Silicon Dioxide, Sodium Lauryl Sulfate and |
| | Sodium Stearyl Fumarate |

### Safety Assessments

The primary objective of the study was to assess the safety and tolerability of Compound 1. Safety was determined by evaluating physical examinations, vital signs, ECGs, clinical laboratory parameters, and AEs.

If deemed necessary, additional safety measurements were performed at the discretion of the PI. The Sponsor Medical Monitor was available for consultation for any potential safety issues throughout the study.

### Physical Examinations

Body height (centimeters) and body weight (kilograms) were measured.

The targeted physical examination included, at a minimum, assessment of the cardiovascular, respiratory, gastrointestinal, musculoskeletal, and neurological systems. Height (at screening only) and weight were also measured and recorded. BMI was calculated at screening and clinic admission only. A licensed physician or qualified designee examined each participant. Physical examinations may be performed at various unscheduled timepoints if deemed necessary by the PI.

Any abnormal findings during the screening period were recorded as medical history.

The PI or qualified designee should pay attention to clinical signs related to previous illnesses.

### Vital Signs

Vital signs were measured with the participant in a supine position after 5 minutes rest in a quiet setting without distractions (e.g., television, cell phones). In instances where the safety ECG coincides with defined timepoints for cardiodynamics ECG extractions, vital signs were taken after at least a 10-minute rest. Vitals included measurement of temperature (oral or tympanic), systolic and diastolic blood pressure, heart rate, and respiratory rate.

Blood pressure and heart rate measurements were assessed with an automated device. Manual techniques were used only if an automated device is not available. For blood pressure and heart rate, 3 readings were taken. The eligibility of the participant at screening was determined using the average of the three readings.

Vital signs were measured at Day −1. For blood pressure and heart rate, three readings should be taken and recorded. Eligibility should be confirmed using the average of the three readings.

On Day 1 and Day 2, vital signs are to be obtained within 30 minutes prior to dosing. Post-dose vital signs are to be taken within 30 minutes of the designated timepoints in the Schedule of Activities (i.e., 2, 4, 8, and 24 hours post-dose).

Vitals were also measured on the final day of study drug administration and SFU (see SoA). For blood pressure and heart rate, three readings should be taken and recorded.

Additional measurements should be taken as deemed necessary by the PI.

### Electrocardiograms

For study conduct, ECGs were classified as Safety ECGs or Cardiodynamics ECGs, and were performed.

### Standard 12-Lead ECGs (Safety ECGs)

12-lead ECGs were performed. Timing and recording technique for ECGs were standardized for all participants. All safety ECGs following study drug administration on Day 1 were obtained and recorded in triplicate.

### Holter Monitoring (Cardiodynamics ECGs)

Timing and recording technique for ECGs were standardized for all participants involved with cardiodynamics monitoring. Holter monitoring was performed to collect continuous 12-lead ECG data from 2 hours pre-dose to 24 hours post-dose on Days 1 and 14. During this period, at defined timepoints generally paired with PK blood draws (with the exception of pre-dose, for which cardiodynamics monitoring requires 3 independent pre-dose measurements at 60, 45, and 30 minutes pre-dose), ECG extractions were performed by a third-party vendor using the data from the Holter monitor.

### Clinical Safety Laboratory Assessments

Clinical safety laboratory assessments were made as summarized above.

The PI or qualified must review the laboratory report, document this review, and record any clinically relevant changes occurring during the study.

Laboratory tests with values considered clinically significantly abnormal during participation in the study should be repeated until the values return to the participant's baseline or are no longer considered clinically significant by the PI.

If such values do not return to normal/baseline within a period of time judged reasonable by the PI, the etiology should be investigated, and the Sponsor notified.

Non-protocol specified laboratory assessments requiring a change in participant management or considered clinically significant by the PI (e.g., resulting in an AE) must also be recorded in the CRF.

### Drug Induced Liver Injury (DILI)

Events meeting the definition for Drug-induced Liver Injury (DILI):
Alanine aminotransferase (ALT) or aspartate aminotransfcrase (AST) >3×ULN and total bilirubin (TBL) >2×ULN in the presence of normal alkaline phosphatase (ALP), requires immediate enhanced monitoring (of all liver tests, minimally: ALT, AST, ALP, and TBL and well as prothrombin time (PT)/INR) and an evaluation for cause of liver test abnormalities (FDA 2009).
ALT or AST>3×ULN with the appearance of fatigue, nausea, vomiting, right upper quadrant pain or tenderness, fever, rash, and/or eosinophilia (>5%) (FDA 2009).

The Medical Monitor should be contacted immediately. Additional medical management considerations (for the specific participant and possibly extending to the broader cohort) while the evaluation is ongoing should be discussed.

The following adverse events of special interest require expedited reporting to Sponsor, the PI should inform the Sponsor within 24 hours of learning of the following events.
Grade >2 cytopenia
Platelets less than 75000/mm3
WBC less than 3000/mm3
Neutrophils less than 1500/mm3
Lymphocytes less than 800/mm3
Hemoglobin less than 10 g/dL
Grade >3 elevation in creatine phosphokinase (CPK) [>5×ULN]

### Pharmacokinetic Measurements

Blood samples of approximately 5 mL were collected for measurement of plasma concentrations of Compound 1 and its metabolites.

The actual date and time (24-hour clock time) of each sample was recorded.

Samples were used to evaluate the PK of Compound 1. Each sample was divided into 2 aliquots (1 each for PK and a back-up). Samples collected for analyses of plasma concentration may also be used to evaluate metabolite formation or safety aspects related to concerns arising during or after the study.

Pharmacokinetic blood samples were collected as follows.

### PK Sampling

| Timepoint | Allowable Windows | |
|---|---|---|
| Pre-dosing (Day 1, 8 and 14) | ±30 | mins |
| 0.5 hour post-dose (Day 1 and 14) | ±5 | mins |
| 1 hour post-dose (Day 1 and 14) | ±5 | mins |
| 2 hours post-dose (Day 1 and 14) | ±5 | mins |
| 3 hours post-dose (Day 1 and 14) | ±10 | mins |
| 4 hours post-dose (Day 1 and 14) | ±30 | mins |
| 6 hours post-dose (Day 1 and 14) | ±60 | mins |
| 8 hours post-dose (Day 1 and 14) | ±60 | mins |
| 12 hours post-dose (Day 1 and 14) | ±60 | mins |
| 24 hours (Day 2 and 15) post-dose | ±60 | mins |
| 48 hours post-dose (Day 3 and 16) | ±120 | mins |
| 72 hours post-dose (Day 4 and 17) | ±120 | mins |

| | |
|---|---|
| End of Study (if early termination) | Not applicable |

Urine samples were collected from all participants in Cohort 2 at Day 1 pre-dosing and at Day 14 as follows:

Time Intervals

| Pre-dosing | Record actual time |
|---|---|
| 0-8 hours post-dose | Record total volume voided, start and stop |
| | time of collection |
| 8-16 hours post-dose | Record total volume voided, start and stop |
| | time of collection |
| 16-24 hours post-dose | Record total volume voided, start and stop |
| | time of collection |

### Pharmacodynamics

As a measure of pharmacodynamic response to treatment with Compound 1, whole-blood samples of ˜1 mL were collected from selected timepoints directly into provided TruCulture (Myriad RBM) whole blood assay collection tubes. Collection occurred within a window of ±30 minutes of the specified timepoint. These samples were incubated at the CRU ex-vivo at 37° C. for ˜24 hours and the levels of IFNγ produced in response to cytokine stimulation from the cell supernatant were quantified using appropriate immunoassays.

### Example 4: Phase 2b Study in Subjects with Plaque Psoriasis

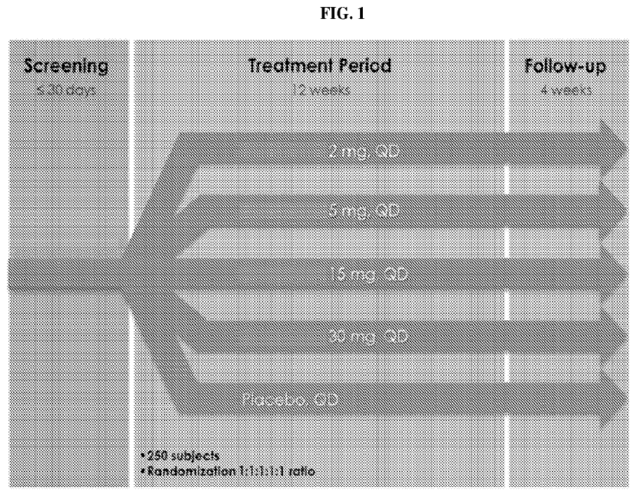

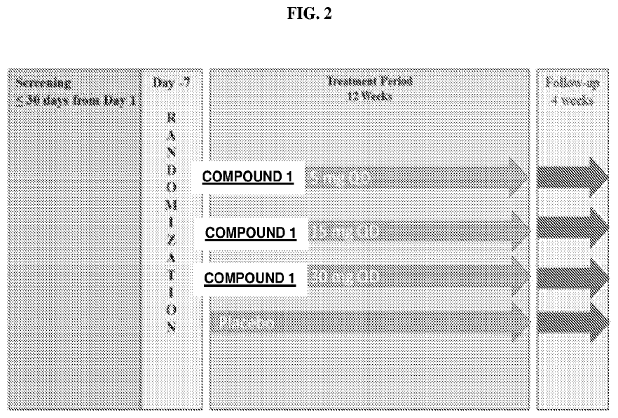

The present study was conducted according to FIG. 1. Approximately 250 subjects will be randomized in this study (approximately 50 subjects/arm). The maximum study duration per subject is approximately 20 weeks, including up to 30 days for the screening period, a 12-week treatment period, and a 4-week safety follow-up period. Compound 1 at doses of 2 mg, 5 mg, 15 mg, or 30 mg, or placebo will be orally administered once daily (QD) for 12 weeks. Compound 1 will be available in 2 mg, 5 mg, and 15 mg strength capsules. Matching placebo will be identical to Compound 1 but will not contain the active ingredient. Subjects will be randomized in a 1:1:1:1:1 ratio.

| Abbreviation or Specialist Term | Explanation |
|---|---|
| AE | adverse event |
| ADL | activities of daily living |
| AESI | adverse event of special interest |
| ALT | alanine aminotransferase |
| anti-HBC | antibody to hepatitis B core antigen |
| aPTT | activated partial thromboplastin time |
| AST | aspartate aminotransferase |
| AUC | area under the concentration versus time curve |
| AUC % | percentage of the area under the concentration versus time curve |
| AUC0-inf | area under the plasma concentration versus time curve from |
| | time 0 to infinity |
| AUC0-last | area under the plasma concentration versus time curve from |
| | time 0 to the time of last measurable concentration |
| AUC0-tau | area under the plasma concentration versus time curve from |
| | time 0 to the end of the dosing period |
| 13-hCG | 13-human chorionic gonadotropin |
| BMI | body mass index |
| BSA | body surface area |
| BUN | blood urea nitrogen |
| Cavg | average concentration over the dosing interval |
| CLr | renal clearance |
| Cmax | maximum observed concentration |
| Cmin | minimum observed concentration |
| CMH | Cochran-Mantel-Haenszel |
| COVID-19 | Coronavirus Disease 2019 |
| CPK | creatine phosphokinase |
| CRO | contract research organization |
| CTCAE | Common Terminology Criteria for Adverse Events |
| Ctrough | trough concentration |
| CV | coefficient of variation |
| CYP3A | cytochrome P450 3A |
| DDI | drug-drug interaction |
| DILI | drug induced liver injury |
| DLQI | Dermatology Life Quality Index |
| ECG | electrocardiogram |
| eCRF | electronic case report form |
| EDC | electronic data capture |
| EOS | end of study |
| EOT | end of treatment |
| ET | early termination |
| F % | oral bioavailability |
| FDA | Food and Drug Administration |
| FE % | fraction of the dose excreted in urine |
| FIH | first in human |
| FSH | follicle-stimulating hormone |
| GCP | Good Clinical Practice |
| GGT | gamma-glutamyl-transferase |
| hADME | human absorption, distribution, metabolism, and excretion |
| HBsAg | hepatitis B surface antigen |
| HBV | hepatitis B virus |
| HCT | hematocrit |
| HCV | hepatitis C virus |
| Hgb | hemoglobin |
| HIV | human immunodeficiency virus |
| IB | Investigator Brochure |
| IC30 | 30% inhibitory concentration |
| IC50 | half maximal inhibitory concentration |
| IC50 | 90% inhibitory concentration |
| ICF | informed consent form |
| ICH | International Council for Harmonisation |
| IL | interleukin |
| IND | Investigational New Drug |
| IFNγ | interferon gamma |
| INR | international normalized ratio |
| IRB | institutional review board |
| ITT | intent-to-treat |
| IV | intravenous |
| IWRS | Interactive Web Response System |
| JAK | Janus kinase |
| LOCF | last observation carried forward |
| LMW | low molecular weight |
| MAD | multiple ascending dose |
| MCH | mean corpuscular hemoglobin |
| MCHC | mean corpuscular hemoglobin concentration |
| MCV | mean corpuscular volume |
| MedDRA | Medical Dictionary for Regulatory Activities |
| MMRM | mixed effect model repeated measures method |
| MPV | mean platelet volume |
| mRNA | messenger ribonucleic acid |
| N/A | not applicable |
| NCI | National Cancer Institute |
| NMSC | nonmelanoma skin cancer |
| NOAEL | no observable adverse effect level |
| NRS | numeric rating scale |
| PASI | Psoriasis Area and Severity Index |
| PASI-100 | 100% improvement from baseline in Psoriasis Area and Severity |
| | Index |
| PASI-50 | 50% improvement from baseline in Psoriasis Area and Severity |
| | Index |
| PASI-75 | 75% improvement from baseline in Psoriasis Area and Severity |
| | Index |
| PASI-90 | 90% improvement from baseline in Psoriasis Area and |
| | Severity Index |
| PBPK | physiologically based pharmacokinetic |
| PCR | polymerase chain reaction |
| PD | pharmacodynamic |
| PDE4 | phosphodiesterase Type 4 |
| PGA | Physician's Global Assessment |
| PK | pharmacokinetic |
| PLT | platelets |
| PP | per-protocol |
| PT | prothrombin time |
| PUVA | psoralen and ultraviolet A |
| QC | quality control |
| QD | once daily |
| RBC | red blood cell (count) |
| REB | research ethics board |
| SAD | single ascending dose |
| SAE | serious adverse event |
| SAP | statistical analysis plan |
| SD | standard deviation |
| SDD | spray dried dispersion |
| SOC | system organ class |
| sPGA | static Physician's Global Assessment |
| STAT | signal transducers and activators of transcription |
| t½ | terminal half-life |
| TB | tuberculosis |
| TEAE | treatment-emergent adverse event |
| Th | T helper |
| Tmax | time to maximum observed concentration |
| TNF | tumor necrosis factor |
| TPGS | d-α-tocopheryl polyethylene glycol 1000 succinate |
| TYK2 | tyrosine kinase 2 |
| UV | ultraviolet |
| WBC | white blood cell (count) |
| WHO-DD | World Health Organization - Drug Dictionary |
| WOCBP | women of childbearing potential |

### The Primary Objective is:

To assess the efficacy of Compound 1 orally administered QD at 2 mg, 5 mg, 15 mg, or 30 mg for 12 weeks in subjects with moderate to severe plaque psoriasis

### The Secondary Objectives are:

To assess the safety and tolerability of Compound 1 orally administered QD at 2 mg, 5 mg, 15 mg, or 30 mg for 12 weeks in subjects with moderate to severe plaque psoriasis
To evaluate the plasma concentration of Compound 1 orally administered QD at 2 mg, 5 mg, 15 mg, or 30 mg in subjects with moderate to severe plaque psoriasis

### The Exploratory Objectives are:

To assess the effects of Compound 1 on joint pain in subjects with moderate to severe plaque psoriasis and concomitant psoriatic arthritis
To evaluate the effects of Compound 1 on cytokines and other inflammatory biomarkers in blood in subjects with moderate to severe plaque psoriasis

### Endpoints:

### Primary Efficacy Endpoint:

Proportion of subjects achieving at least a 75% improvement from baseline in Psoriasis Area and Severity Index (PASI-75) at Week 12

### Secondary Efficacy Endpoints:

Proportion of subjects achieving a Physician Global Assessment (PGA) of clear (0) or almost clear (1) at Week 12
Proportion of subjects achieving at least a 90% improvement from baseline in Psoriasis Area and Severity Index (PASI-90) at Week 12
Proportion of subjects achieving a 100% improvement from baseline in Psoriasis Area and Severity Index (PASI-100) at Week 12
Change from baseline in Dermatology Life Quality Index (DLQI) at

### Week 12 Exploratory Efficacy Endpoints:

Proportion of subjects achieving at least a 50% improvement from baseline in Psoriasis Area and Severity Index (PASI-50) at Weeks 2, 4, 8, and 12
Proportion of subjects achieving PASI-75 at Weeks 2, 4, and 8
Proportion of subjects achieving PASI-90 at Weeks 2, 4, and 8
Proportion of subjects achieving PASI-100 at Weeks 2, 4, and 8
Change from baseline in Psoriasis Area and Severity Index (PASI) at Weeks 2, 4, 8, and 12
Percent change from baseline in PASI at Weeks 2, 4, 8, and 12
Change from baseline in PGA at Weeks 2, 4, 8, and 12
Proportion of subjects achieving a PGA of clear (0) or almost clear (1) at Weeks 2, 4, and 8
Proportion of subjects with at least a 2-grade decrease from baseline in PGA at Weeks 2, 4, 8, and 12
Change from baseline in body surface area (BSA) at Weeks 2, 4, 8, and 12
Change from baseline in pruritus numeric rating scale (NRS) at Weeks 2, 4, 8, and 12
Proportion of subjects with a baseline pruritus NRS of 4 or greater achieving at least a 4-point decrease from baseline in pruritus NRS at Weeks 2, 4, 8, and 12
Change from baseline in Dermatology Life Quality Index (DLQI) at Weeks 4 and 8
Change from baseline in pain NRS at Weeks 2, 4, 8, and 12 for subjects with concomitant psoriatic arthritis

### Secondary Safety Endpoints:

Incidence of adverse events (AEs)
Changes in vital signs, clinical laboratory parameters, and electrocardiograms (ECGs)

### Secondary Pharmacokinetic (PK) Endpoint:

1. Measurement of plasma concentrations of Compound 1 in subjects receiving active treatment Exploratory pharmacodynamic (PD) endpoints:
2. Quantification of skin biomarkers (immune cell infiltration and mRNA expression levels) in lesional and nonlesional skin
3. Quantification of circulating cytokines and other inflammatory biomarkers

Study Design: This is a Phase 2b, randomized, multicenter, double-blind, placebo-controlled, multiple-dose study designed to evaluate the efficacy, safety, and tolerability of Compound 1 in subjects with moderate to severe plaque psoriasis. This study will also evaluate the plasma concentrations of Compound 1 and explore the immune response (in blood and skin) to Compound 1 in subjects with moderate to severe plaque psoriasis. Approximately 250 male and female subjects, aged 18 to 70 years (inclusive), with moderate to severe plaque psoriasis will be randomized in this study. To be eligible for the study, the subjects will need to have a history of plaque psoriasis for at least 6 months prior to the screening visit. In addition, the subjects will need to have the following characteristics at screening and on Day 1: PASI score of at least 12, PGA score of at least 3, and BSA involved with plaque psoriasis of at least 10%.

All subjects will read and sign an informed consent form (ICF) prior to any screening procedures being performed. Subjects who fulfill all of the inclusion criteria and none of the exclusion criteria will be included into the study. During a screening period of no longer than Day −30 to Day −1, subjects will be randomized (on Day −7) to receive either one of the four doses of Compound 1 (2 mg, 5 mg, 15 mg, or 30 mg), or placebo on Day 1. The goal is to have approximately 50 subjects randomized per treatment group (1:1:1:1:1 ratio) on Day 1. During the treatment period, Compound 1 (2 mg, 5 mg, 15 mg, or 30 mg) or placebo will be orally administered QD for 12 weeks. The 12-week treatment period will be followed by a 4-week safety follow-up period. For scheduled study visits, subjects will come to the study site on 8 occasions: screening, Day 1, and Weeks 1, 2, 4, 8, 12 (end of treatment [EOT]), and 16 (end of study [EOS]/early termination visit [ET]). Efficacy will be assessed using PASI, PGA, BSA involved with plaque psoriasis, pruritus NRS, and pain NRS for subjects with concomitant psoriatic arthritis. Quality of life will be evaluated using DLQI. Safety will be assessed by collecting AEs, recording vital signs, performing physical examinations, and evaluating clinical laboratory and ECGs results.

Blood samples will be collected to measure plasma levels of Compound 1 as follows:
On Day 1 prior to dosing and 1 hour (±5 min) postdosing;
At Week 4 prior to dosing, 1 hour (±5 min) postdosing, and 4 hours (±10 min) postdosing;
At Week 8 prior to dosing;
At Week 12 anytime (no study treatment administration at this visit).
At ET visit anytime (if ET visit is planned before Week 12 visit).

Urine samples will be collected on Day 1, and at Weeks 4, 12, and 16 and may be used to evaluate the effect of Compound 1 on exploratory biomarkers. Blood samples will be collected on Day 1, and at Weeks 4 and 12 to evaluate the effect of Compound 1 on circulating inflammatory biomarkers. In a subset of subjects who consent to the procedure, the effect of Compound 1 on skin biomarkers will be evaluated by collecting three or four optional skin biopsies. Two 5-mm punch biopsies (one from lesional skin and one from adjacent nonlesional skin) will be collected on Day 1, and one 5-mm punch biopsy will be collected from the same lesional skin (outside the scar of the previous biopsy, at least 1 cm away from the previous scar, even if the lesion has cleared) at Week 12. In addition, one 5-mm punch biopsy will be collected from the same lesional skin (outside the scar of the previous biopsy, at least 1 cm away from the previous scar) at Week 4 in subjects who consent to four skin biopsies. Photographs of biopsied areas will be performed.

In a subset of subjects who consent to the procedure, the effect of Compound 1 on skin biomarkers will be evaluated by collecting tape strips. Skin tape strips will be collected from lesional skin and from adjacent nonlesional skin on Day 1, and from same lesional skin at Week 12. At certain study sites, in a subset of subjects who consent, optional medical photographs of full body, front and back, will be taken to illustrate the outcome of the study.

Inclusion/Exclusion Criteria: In order to be eligible to participate in this study, a subject must meet all of the following criteria, either at the screening and Day 1 visits or only at one of the specified visits (screening or Day 1) as noted in the criterion:
Male or female subject aged 18 to 70 years, inclusive, at the time of consent.
Subject has a history of plaque psoriasis for at least 6 months prior to the screening visit.
Subject had no significant flare in psoriasis for at least 3 months before screening (information obtained from medical chart or subject's physician, or directly from the subject).
Subject has moderate to severe plaque psoriasis as defined by a PASI score of at least 12 and a PGA score of at least 3 at screening and Day 1.
Subject has plaque psoriasis covering 10% of his or her total BSA at screening and Day 1.
Subject must be a candidate for phototherapy or systemic therapy.
For female subjects of childbearing potential involved in any sexual intercourse that could lead to pregnancy: the subject must agree to use a highly effective contraceptive method from at least 4 weeks prior to Day 1 until at least 4 weeks after the last study product administration. Highly effective contraceptive methods include hormonal contraceptives (e.g., combined oral contraceptive, patch, vaginal ring, injectable, or implant), intrauterine devices or intrauterine systems, vasectomized partner(s) (provided vasectomy was performed at least 4 months prior to screening), bilateral tubal ligation or occlusion, or double barrier methods of contraception (e.g., male condom with cervical cap, male condom with diaphragm, and male condom with contraceptive sponge) in conjunction with spermicide. Note: Subjects must have been on a stable dose of hormonal contraceptives for at least 4 weeks before Day 1. Note: The above list of contraceptive methods does not apply to subjects who are abstinent for at least 4 weeks before Day 1 and will continue to be abstinent from penile-vaginal intercourse throughout the study. The reliability of sexual abstinence needs to be evaluated in relation to the duration of the clinical study, and the preferred and usual lifestyle of the subject. Periodic abstinence (calendar, symptothermal, postovulation methods) is not acceptable. Note: A female subject of nonchildbearing potential is defined as follows:
   Female subject who has had surgical sterilization (hysterectomy, bilateral oophorectomy, or bilateral salpingectomy);
   Female subject who has had a cessation of menses for at least 12 months prior to the screening visit without an alternative medical cause, and a follicle-stimulating hormone (FSH) test confirming nonchildbearing potential (refer to laboratory reference ranges for confirmatory levels).
Female subjects of childbearing potential have had a negative serum pregnancy test at screening and negative urine pregnancy test at Day 1.
For male subjects involved in any sexual intercourse that could lead to pregnancy, subject must agree to use one of the highly effective contraceptive methods listed in Inclusion Criterion 6, from Day 1 until at least 12 weeks after the last study product administration. If the female partner of a male subject uses any of the hormonal contraceptive methods listed above, this contraceptive method should be used by the female partner from at least 4 weeks before Day 1 until at least 12 weeks after the last study product administration.
   Note: Male subjects must refrain from donating sperm from Day 1 until at least 12 weeks after the last study product administration.
   Note: No restrictions are required for a male subject who underwent a vasectomy at least 4 months prior to screening and the procedure is documented. If vasectomy procedure is not documented or was performed less than 4 months prior to screening, male subjects must follow the same contraception and sperm donation requirements as for nonvasectomized subjects.
Subject has a body mass index (BMI) within the range of 18 to 38 kg/m², inclusive (BMI=weight [kg]/[height (m)]²), and total body weight >50 kg (110 lb).
Subject is willing to participate and is capable of giving informed consent. Note: Consent must be obtained prior to any study-related procedures.
Subjects must be willing to comply with all study procedures and must be available for the duration of the study.

Exclusion criteria: A subject who meets any of the following criteria at the screening and/or Day 1 visits, as applicable, will be excluded from participation in this study:
1. Subject is a female who is breastfeeding, pregnant, or who is planning to become pregnant during the study.
2. Subject has evidence of erythrodermic, pustular, predominantly guttate psoriasis, or drug-induced psoriasis.
3. Subject has a history of skin disease or presence of skin condition that, in the opinion of the invcstigator, would intcrfcrc with the study asscssmcnts.
4. Subject has immune-mediated conditions commonly associated with psoriasis, such as psoriatic arthritis, uveitis, inflammatory bowel disease, that require systemic treatment (including corticosteroids, immunosuppressants, or biologics).
   Note: Subjects with immune-mediated conditions that do not require systemic treatment may be included in the study. Certain therapies such as NSAIDs may be permitted but should be discussed with the Medical Monitor prior to determination of subject eligibility.
5. Subject has any clinically significant medical condition, evidence of an unstable clinical condition (e.g., cardiovascular, renal, hepatic, hematologic, gastrointestinal, endocrine, pulmonary, immunologic, or local active infection/infectious illness), psychiatric condition, or vital signs/physical/laboratory/ECG abnormality that would, in the opinion of the investigator, put the subject at undue risk or interfere with interpretation of study results.
6. Subject had a major surgery within 8 weeks prior to Day 1 or has a major surgery planned during the study.
7. Subject has a history of Class III or IV congestive heart failure as defined by New York Heart Association Criteria.
8. Subject has been hospitalized in the past 3 months for asthma, has ever required intubation for treatment of asthma, currently require oral corticosteroids for the treatment of asthma, or has required more than one short-term (≤2 weeks) course of oral corticosteroids for asthma within 6 months prior to Day 1.
9. Subject has a history of cancer or lymphoproliferative disease within 5 years prior to Day 1. Subjects with successfully treated nonmetastatic cutaneous squamous cell or basal cell carcinoma and/or localized carcinoma in situ of the cervix are not to be excluded.
10. Subject has a history of fever, inflammation, or systemic signs of illness suggestive of systemic or invasive infection within 4 weeks prior to Day 1.
11. Subject has an active bacterial, viral, fungal, mycobacterial infection, or other infection (including TB or atypical mycobacterial disease), or any major episode of infection that required hospitalization or treatment with intravenous antibiotics within 12 wecks prior to Day 1, or oral antibiotics within 4 weeks prior to Day 1.
12. Subject has a history of chronic or recurrent infectious disease, including but not limited to chronic renal infection, chronic chest infection, recurrent urinary tract infection, fungal infection (with the exception of superficial fungal infection of the nailbed), or infected skin wounds or ulcers.
13. Subject has a history of an infected joint prosthesis or has received antibiotics for a suspected infection of a joint prosthesis if that prosthesis has not been removed or replaced.
14. Subject has active herpes infection, including herpes simplex 1 and 2 and herpes zoster (demonstrated on physical examination and/or medical history) within 8 weeks prior to Day 1.
15. Subject has a history of known or suspected congenital or acquired immunodeficiency state or condition that would compromise the subject's immune status in the opinion of the investigator (e.g., history of splenectomy, primary immunodeficiency).
16. Subject has positive results for hepatitis B surface antigens (HBsAg), antibodies to hepatitis B core antigens (anti-HBc), hepatitis C virus (HCV), or human immunodeficiency virus (HIV).
17. Subject has clinical or laboratory evidence of active or latent tuberculosis (TB) infection at screening. Note: Subjects with an history of active or latent TB will not be included in the study, unless documentation of prior and complete anti-TB treatment, appropriate in duration and type according to current local country guidelines, can be provided. Note: Subject will be evaluated at screening for latent TB infection with a QuantiFERON-TB Gold (QFT) test. Latent TB is defined as a positive QFT test or two successive indeterminate QTF tests at screening.
18. Subject with any of the following laboratory values at the screening visit:

$$Alanine aminotransferase ( ALT ) or$$

$$aspartate aminotransferase ( AST ) values \geq 3 times the upper limit of normal ( ULN ) ;$$

$$Hemoglobin < 11. g / dL ( < 110. g / L ) ;$$

$$White blood cell count < 3.5 \times {10}^{9} / L ( < 3500 / {mm}^{3} ) ;$$

$$Absolute neutrophil count of < 1.8 \times {10}^{9} / L ( < 1 \; 800 / {mm}^{3} ) ;$$

$$Absolute lymphocyte count < 1. \times {10}^{9} / L ( < 1000 / {mm}^{3} ) ;$$

$$Platelet count < 100 \times {10}^{9} / L ( < 100 , 000 / {mm}^{3} ) ;$$

$$Total bilirubin > 2 times the ULN .$$

19. Subjects who have given >50 ml of blood or plasma within 30 days of screening or >500 mL of blood or plasma within 56 days of screening (during a clinical study or at a blood bank donation).
20. Subject has used any topical medication that could affect psoriasis (including corticosteroids, retinoids, vitamin D analogues [such as calcipotriol], JAK inhibitors, or tar) within 2 weeks prior to Day 1.
21. Subject has used any systemic treatment that could affect psoriasis (including oral, intravenous, intramuscular, or intralesional corticosteroids; oral retinoids; immunosuppressive/immunomodulating medication; methotrexate; cyclosporine; oral JAK inhibitors; or apremilast) within 4 weeks prior to Day 1. Note: Intranasal corticosteroids and inhaled corticosteroids are allowed. Eye and ear drops containing corticosteroids are also allowed.
22. Subject has received any ultraviolet (UV)—B phototherapy (including tanning beds) or excimer laser within 4 weeks prior to Day 1.
23. Subject has had psoralen and ultraviolet A (PUVA) treatment within 4 weeks prior to Day 1.
24. Subject has received any live-attenuated vaccine within 4 weeks prior to Day 1 or plans to receive a live-attenuated vaccine during the study and up to 4 weeks or 5 half-lives of the study product, whichever is longer, after the last study product administration. Note: Nonlive-attenuated vaccines for Coronavirus Disease 2019 (COVID-19) (e.g., RNA-based vaccines, inactivated adenovirus-based vaccines, protein-based vaccines) are allowed during the study. The study site should follow local guidelines related to COVID-19.
25. Subject is currently receiving a nonbiological investigational product or device or has received one within 4 weeks prior to Day 1.
26. Subject has received any marketed or investigational biological agent within 12 weeks or 5 half-lives (whichever is longer) prior to Day 1 (except those listed in Exclusion Criterion 27 and 28 that are to be excluded for 6 months).
27. Subject was previously enrolled in any study with Compound 1.
28. Subject has a history of lack of response to any therapeutic agent targeting interleukin (IL)-12, IL-17, and/or IL-23 (e.g., ustekinumab, secukinumab, ixekizumab, brodalumab, guselkumab, tildrakizumab, risankizumab) at approved doses after at least 12 weeks of therapy, and/or received one of these therapies within 6 months prior to Day 1.
29. Subject has received rituximab or other immune-cell depleting therapy within 6 months.
30. Subject is currently being treated with strong or moderate cytochrome P450 3A (CYP3A4) inhibitors (such as itraconazole) or has received moderate or strong CYP3A4 inhibitors within 4 weeks prior to Day 1.
31. Subject is currently being treated with terbinafine or has received terbinafine within 4 weeks prior to Day 1.
32. Subject has consumed grapefruit within 1 week prior to Day 1.
   Note: Consumption of grapefruit must be avoided during the treatment period and for at least 1 week after last dose administration.
33. Subject has used tanning booths within 4 weeks prior to Day 1, has had excessive sun exposure, or is not willing to minimize natural and artificial sunlight exposure during the study. Note: Use of sunscreen products and protective apparel are recommended when sun exposure cannot be avoided.
34. Subject has a known or suspected allergy to Compound 1 or any component of the investigational product, or any other significant drug allergy (such as anaphylaxis or hepatotoxicity).
35. Subject has a known history of clinically significant drug or alcohol abuse in the last year prior to Day 1.
36. For subjects consenting to biopsy collection only:
   Subject has a history of an allergic reaction or significant sensitivity to lidocaine or other local anesthetics.
   Subject has a history of hypertrophic scarring or keloid formation in scars or suture sites.
   Subject has taken anticoagulant medication, such as heparin, low molecular weight (LMW)-heparin, warfarin, or antiplatelet agents (except low-dose aspirin ≤81 mg which will be allowed), within 2 weeks prior to Day 1, or has a contraindication to skin biopsies. Nonsteroidal anti-inflammatory drugs will not be considered antiplatelet agents and will be allowed.

Categorical variables will be presented in tables as frequencies and percentages. Continuous variables will be summarized in tables and will include the number of subjects, mean, standard deviation (SD), median, minimum, and maximum. Further details regarding the efficacy and safety variable definitions, analyses strategy, statistical justification, and techniques for handling missing values (if applicable) will be detailed in a separate statistical analysis plan (SAP) that will be prepared before the database is locked and any analyses are undertaken. Any deviation(s) from the SAP will be described and justified in the final Clinical Study Report, as appropriate.

All statistical tests will be two-sided and will be performed with a significant level of 0.05, unless otherwise specified in the SAP. No adjustment to alpha will be made to account for multiple testing between treatment groups.

Efficacy Analyses: The primary endpoint can be translated as a responder analysis, where a subject will be classified as responder if he or she achieves PASI-75 at Week 12. The comparison between groups for the primary endpoint will be done using a Cochran-Mantel-Haenszel (CMH), with prior treatment with biologics included as a stratification factor. The primary efficacy analysis will be performed on the intent-to-treat (ITT) analysis set, while the per-protocol (PP) analysis set will be used as a sensitivity analysis.

The secondary endpoints involving proportions of subjects will be analyzed using the same approach (CMH test) as described for the primary efficacy analysis, at each time point and based on the ITT analysis. The continuous secondary endpoints involving absolute change from baseline will be analyzed using a mixed effect model repeated measures method (MMRM) based on the ITT analysis set only. The model will include treatment, visit, treatment-by-visit interaction, and prior treatment with biologics as fixed effects, and baseline score as a covariate.

Safety Analyses: All safety analyses will be conducted using the safety analysis set. No inferential statistics will be performed on safety variables. Adverse events and serious adverse events (SAEs) will be presented and tabulated according to the Medical Dictionary for Regulatory Activities (MedDrA) classification by treatment group. Descriptions of AEs will include the start date, the stop date (if it resolved), the severity and seriousness of the AE, the causality of the AE to study product, action taken with respect to the study product, and the outcome.

Reported AEs will be summarized by the number of subjects reporting the events, as well as by system organ class, preferred term, reported verbatim severity, seriousness, and investigator's assessment of the relationship to study product. For the summary of AEs by severity, each subject will be counted only once within a system organ class or a preferred term by using the AEs with the highest intensity within each category for each analysis. For the summary of AEs by relationship to study product, each subject will be counted only once within a system organ class or a preferred term by using the AEs with the greatest reported relationship within each category. For the summary of AEs by relationship to study product and severity, each subject will be counted only once within a system organ class or a preferred term by using (1) the greatest reported relationship followed by (2) the highest reported intensity.

All information pertaining to AEs noted during the study will be listed by treatment group, subject, detailing verbatim, system organ class, preferred term, start date, stop date, intensity, outcome, action taken with respect to study product, and relationship to study product. The AE onset will also be shown relative (in number of days) to the day of study product administration. Serious adverse events will be tabulated by treatment group, relationship to the test article, and a reference to the occurrence of the SAEs to the relative day of dosing. Similar listings will be provided for the SAEs and AEs leading to the discontinuation from the study.

Results from vital signs, laboratory analyses, and ECGs will be tabulated by treatment group and visit using descriptive statistics. The observed value at each visit, as well as the change from baseline will be presented. Shift tables describing shifts to out-of-normal range will be provided for clinical laboratory results, and normal-abnormal shift tables may be provided for vital signs.

Concomitant medications will be coded with the World Health Organization-Drug Dictionary (WHO-DD) and listed by subject. Summary of medications will also be tabulated.

Pharmacokinetic Analyses: Concentration data will be listed per subject and summarized descriptively per dose.

Pharmacodynamic Analyses: Analyses of urine, blood, and skin biomarker levels will be described in a separate analysis plan.

Other Analyses: Descriptive summaries of subject disposition and baseline characteristics (including demographic data and prior concomitant therapy) will be presented by treatment group. In addition, a list of subjects who discontinued from the study along with discontinuation reason will be provided. Protocol deviations will be summarized by treatment and category.

Sample Size Consideration: The sample size determination is based on testing equality of two independent response rates using a 2-sided test with significance level of alpha=0.05 and power 85%. The formula used for the calculation is the same as used in the nQuery® (ie, normal approximation with Fleiss' formula and continuity correction).

Assuming the placebo response rate at end of Week 12 is 10% (proportion of subjects achieving PASI-75), at least one of the Compound 1 dose treatment groups will have a response rate at least 40%, and after adjustment of 15% dropout rate, a total of 250 subjects (50 per treatment group) will be randomized in the study. Fifty subjects per treatment group, with 1-sided, 2-sample Fisher's exact test at significant level 0.05, will provide at least approximately 90% power to detect at least 30% difference in the response rate in PASI-75 between any two treatment groups.

Results: Baseline Characteristics. Demographics of study subjects and their baseline disease characteristics are presented in the following Table:

| | | | Compound 1 | | Compound 1 | | Compound 1 | | Compound 1 | |
|---|---|---|---|---|---|---|---|---|---|---|
| | Placebo | | 2 mg QD | | 5 mg QD | | 15 mg QD | | 30 mg QD | |
| | (n = 52) | | (n = 50) | | (n = 52) | | (n = 53) | | (n = 52) | |
| Age, mean (SD), | 48.8 | (12.7) | 45.8 | (14.2) | 45.1 | (13.6) | 46.2 | (13.0) | 48.5 | (11.4) |
| years | | | | | | | | | | |
| Male, n (%) | 31 | (59.6) | 38 | (76.0) | 41 | (78.8) | 34 | (64.2) | 33 | (63.5) |
| Race, n (%) | 44 | (84.6) | 43 | (86.0) | 40 | (76.9) | 46 | (86.8) | 42 | (80.8) |
| White | 5 | (9.6) | 3 | (6.0) | 7 | (13.5) | 2 | (3.8) | 3 | (5.8) |
| Asian | 2 | (3.8) | 4 | (8.0) | 4 | (7.7) | 3 | (5.7) | 4 | (7.7) |
| Black/African | 1 | (1.9) | 0 | | 1 | (1.9) | 2 | (3.8) | 3 | (5.8) |
| American | | | | | | | | | | |
| Other | | | | | | | | | | |
| Weight, mean | 88.4 | (15.8) | 93.9 | (16.7) | 90.4 | (18.7) | 92.7 | (16.8) | 90.0 | (18.3 |
| (SD), kg | | | | | | | | | | |
| BMI, mean (SD), | 31.3 | (5.1) | 31.2 | (5.2) | 30.5 | (5.7) | 32.0 | (4.9) | 30.4 | (5.4) |
| kg/m² | | | | | | | | | | |
| Psoriasis duration, | 12.7 | (10.5) | 13.8 | (10.8) | 14.8 | (10.7) | 17.6 | (14.6) | 17.4 | (11.1) |
| mean (SD), years | | | | | | | | | | |
| PASI score, mean | 18.3 | (8.1) | 18.4 | (6.8) | 18.6 | (6.1) | 15.5 | (4.5) | 17.6 | (6.2) |
| (SD) | | | | | | | | | | |
| DLQI score, mean | 12.4 | (7.0) | 10.3 | (6.2) | 12.8 | (7.5) | 11.9 | (7.1) | 12.5 | (6.9) |
| (SD) | | | | | | | | | | |
| BSA, mean (SD) | 21.3 | (13.6) | 24.9 | (15.5) | 22.6 | (12.1) | 18.3 | (10.3) | 22.2 | (14.3) |
| Bioexperienced, n | 8 | (15.4) | 8 | (16.0) | 8 | (15.4) | 9 | (17.0) | 8 | (15.4) |
| (%) | | | | | | | | | | |

BMI, body mass index;
BSA, body surface area;
DLQI, Dermatology Life Quality Index;
PASI, Psoriasis Area and Severity Index;
QD, once daily;
SD, standard deviation

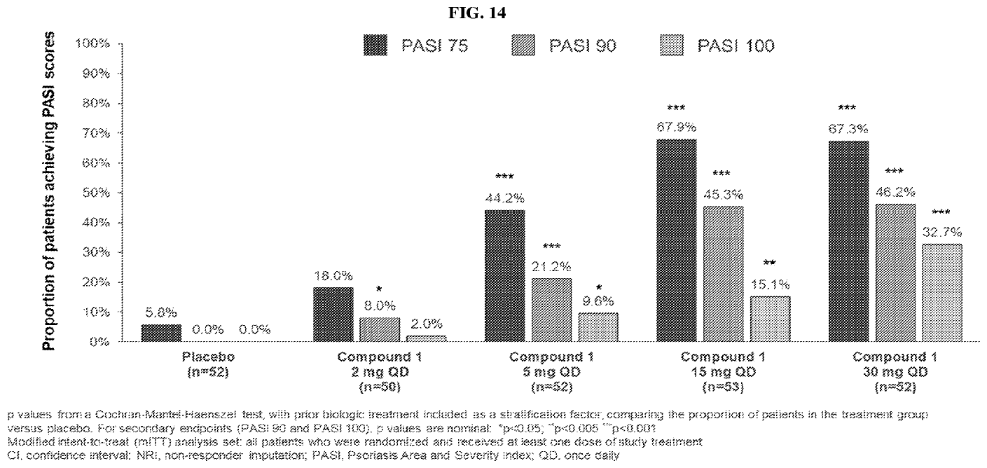

Results: The primary endpoint (PASI 75 response at week 12), was achieved with Compound 1 at doses ≥5 mg, 68% of patients on Compound 115 mg QD and 67% of patients on 30 mg QD achieved PAST 75. Secondary endpoints were also achieved with Compound 1 at doses ≥5 mg: a greater proportion of patients achieved PAST 100 or PGA 0 at the highest dose of Compound 1, and at 30 mg QD dosing, 33% of patients achieved clear skin. More detailed results for the primary endpoint (PAST 75 at week 12), for the modified intent-to-treat (mITT) analysis set, are shown in the following Table. Results are presented graphically in FIG. 13. A graph illustrating the fraction of patients achieving a PAST score of 75, 90, or 100 is shown in FIG. 14.

| | | Compound 1 | Compound 1 | Compound 1 | Compound 1 |
|---|---|---|---|---|---|
| | Placebo | 2 mg QD | 5 mg QD | 15 mg QD | 30 mg QD |
| | (n = 52) | (n = 50) | (n = 52) | (n = 53) | (n = 52) |
| Achieving PASI | 3 | 9 | 23 | 36 | 35 |
| 75 at Week 12* | (5.8) | (18.0) | (44.2) | (67.9) | (67.3) |
| Proportion | — | 12.3 | 38.5 | 61.9 | 61.5 |
| difference (95% CI) | | (0.04, 24.60) | (23.52, 53.40) | (47.56, 76.20) | (47.46, 75.61) |
| Odds ratio (95% | — | 3.73 | 12.73 | 29.01 | 40.11 |
| CI)^{†} | | (0.93, 14.89) | (3.53, 45.99) | (8.53, 98.74) | (10.28, 156.55) |
| p value^{‡} | — | 0.052 | <0.001 | <0.001 | <0.001 |

*Number of patients (percent)
^{†}Adjusted odds ratio using Cochran-Mantel-Haenszel statistics
^{‡}p value from a Cochran-Mantel-Haenszel test, with prior biologic treatment included as a stratification factor, comparing the proportion of patients in the treatment group versus placebo.
Modified intent-to-treat (mITT) analysis set: all subjects who were randomized and received at least one dose of study treatment
CI, confidence interval;
QD, once daily

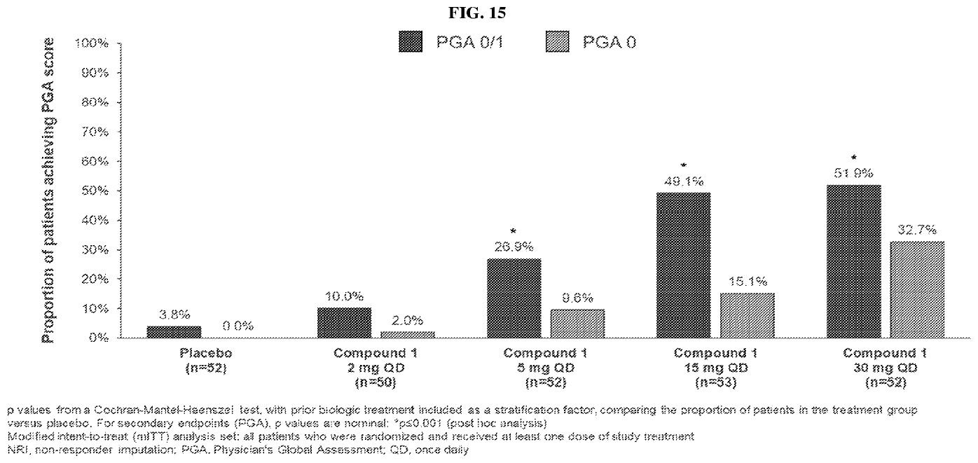

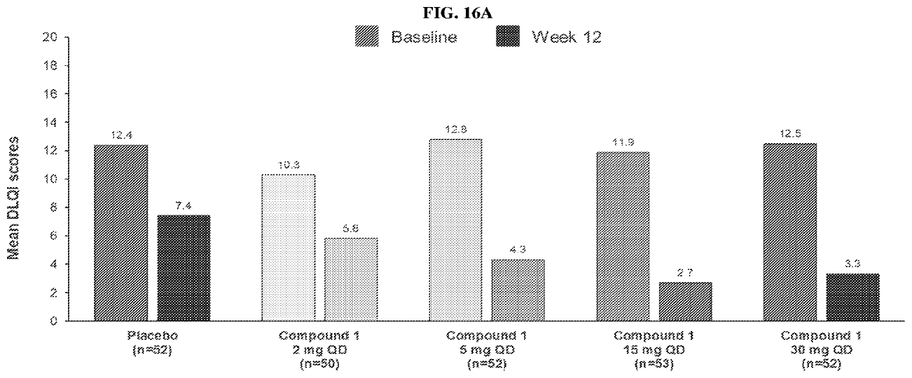

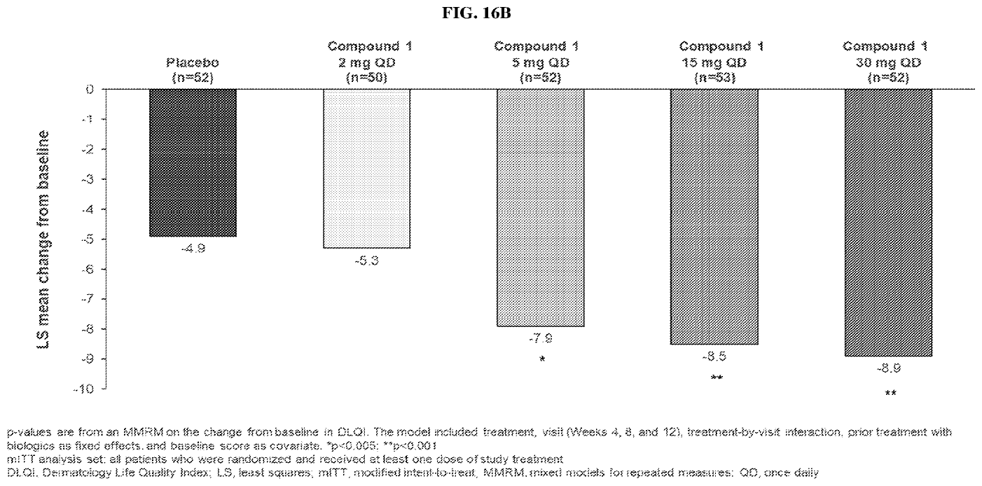

In FIG. 15, the fraction of patients achieving PGA 0/1 at Week 12 is shown. The fractions for PGA 1 and for PGA 0 both increased with increasing dose of Compound 1. The mean Dermatology Life Quality Index (DLQI) score at baseline and 12 weeks, for placebo and the different dose levels of Compound 1 is shown in FIG. 16A; and FIG. 16B shows the change from baseline in DLQI at Week 12.

Safety results are summarized in the following Tables.

### Safety Summary

| | | Compound 1 | Compound 1 | Compound 1 | Compound 1 |
|---|---|---|---|---|---|
| | Placebo | 2 mg QD | 5 mg QD | 15 mg QD | 30 mg QD |
| | (n = 52) | (n = 50) | (n = 52) | (n = 53) | (n = 52) |
| Deaths | 0 | 0 | 0 | 0 | 0 |
| Serious AEs | 0 | 0 | 0 | 1 (2) | 0 |
| AEs | 23 (44) | 31 (62) | 28 (54) | 28 (53) | 31 (60) |
| AEs leading to | 1 (2) | 1 (2) | 1 (2) | 1 (2) | 2 (4) |
| discontinuation* | | | | | |
| Most frequent AEs^{†} | | | | | |
| COVID-19 | 1 (2) | 6 (12) | 4 (8) | 6 (11) | 7 (14) |
| Acne | 0 | 0 | 1 (2) | 3 (6) | 2 (4) |
| Acneiform dermatitis | 0 | 0 | 1 (2) | 1 (2) | 3 (6) |
| Diarrhea | 1 (2) | 3 (6) | 1 (2) | 1 (2) | 0 |

*Adverse events leading to drug discontinuation and early termination in 5 subjects included:
CPK increased (30 mg)
Pericardial effusion and pleural effusion (15 mg)
Tachycardia and syncope (5 mg)
Lymphocyte count decreased (2 mg)
Atrial fibrillation (placebo)
One additional subject (30 mg) permanently discontinued study drug due to an AE of hypertensive urgency, but remained in study.
Number of patients (percent), data rounded up to the nearest integer
*No patients discontinued due to COVID-19.
^{†}AEs reported by ≥3 patients in any treatment group (events elicited by laboratory testing are not included).

Common terminology criteria for adverse events Grade ≥3

| Treatment-emergent | | Compound 1 | Compound 1 | Compound 1 | Compound 1 |
|---|---|---|---|---|---|
| laboratory shifts | Placebo | 2 mg QD | 5 mg QD | 15 mg QD | 30 mg QD |
| CTCAE Grade ≥3^{a, b} | (n = 52) | (n = 50) | (n = 52) | (n = 53) | (n = 52) |
| Neutropenia | 1 (2) | 1 (2) | 0 | 0 | 1 (2) |
| Lymphopenia | 1 (2) | 1 (2) | 0 | 0 | 0 |
| Anemia | 0 | 0 | 0 | 0 | 0 |
| Thrombocytopenia | 0 | 0 | 0 | 0 | 0 |
| CPK elevation | 1 (2) | 0 | 0 | 1 (2) | 1 (2) |
| ALT elevation | 0 | 0 | 0 | 0 | 0 |
| AST elevation | 0 | 0 | 0 | 0 | 0 |
| Creatinine elevation | 0 | 0 | 0 | 0 | 0 |
| Cholesterol elevation, Wk | 0 | 0 | 0 | 0 | 0 |
| 12 | | | | | |
| Triglyceride elevation, | 1 (2) | 1 (2) | 0 | 1 (2) | 1 (2) |
| Wk 12 | | | | | |
| Worsening of proteinuria | 0 | 0 | 0 | 0 | 0 |

^{a}Post-hoc analysis, percent rounded up to nearest integer
^{b}Treatment-emergent and ≥1 grade increase from baseline
ALT, alanine aminotransferase;
AST, aspartate aminotransferase;
CPK, creatine kinase;
CTCAE, common terminology criteria for adverse events;
QD, once daily;
Wk, week

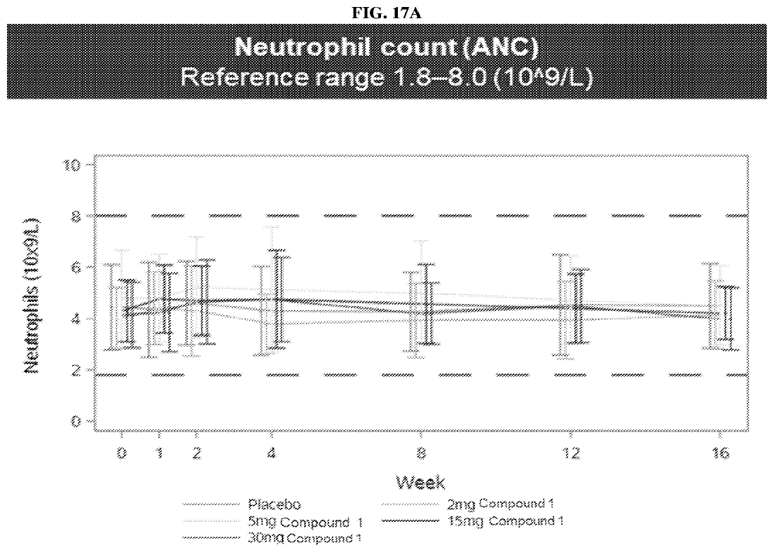

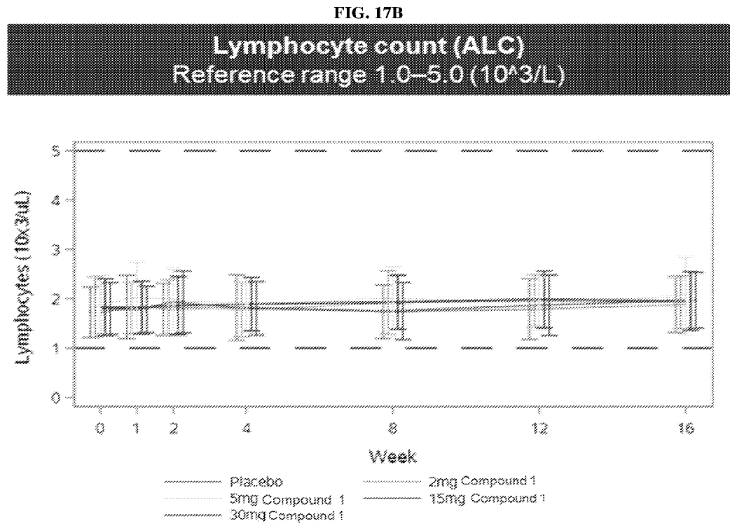

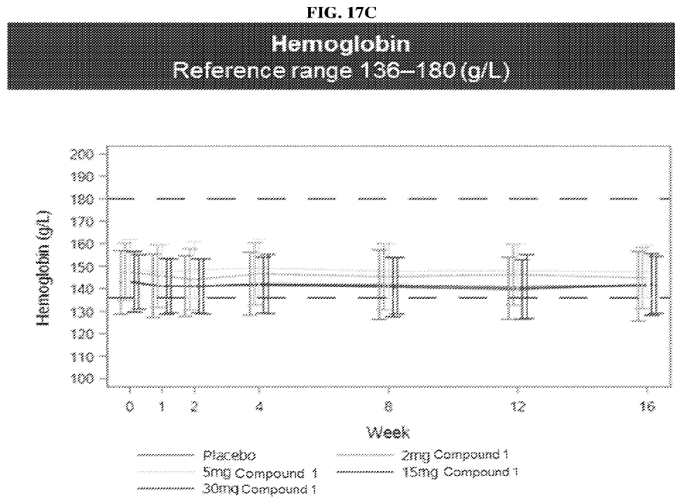

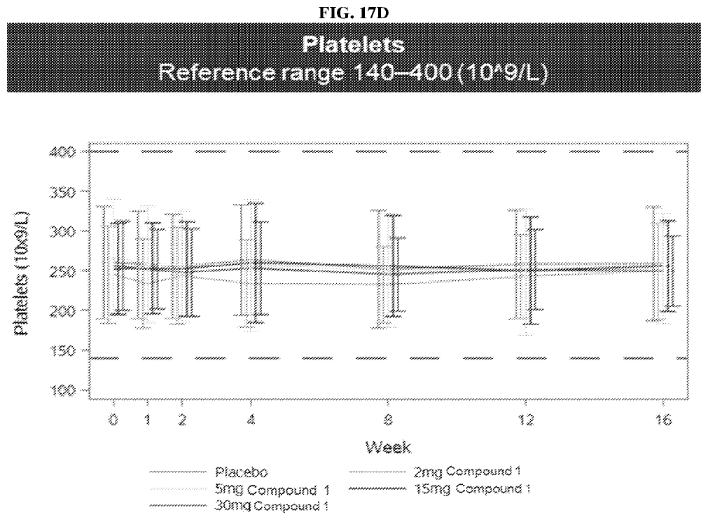

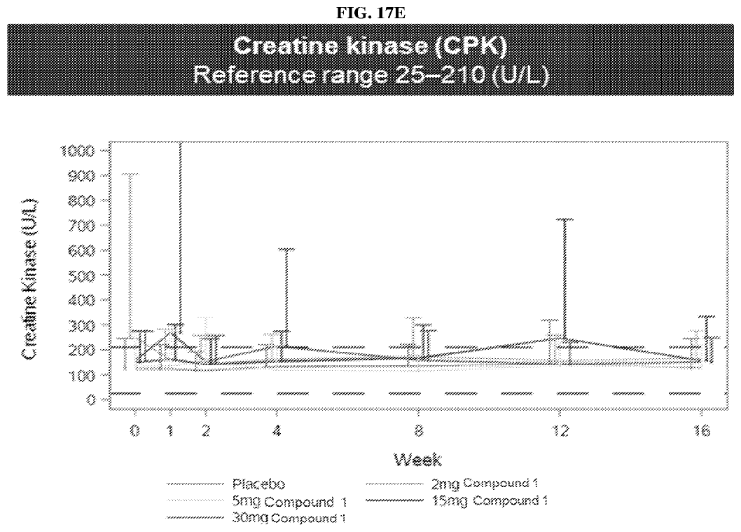

FIGS. 17A-17E show hematological parameters and CPK data collected during the study. Data are mean±standard deviation. Mean lab values and changes from baseline do not reveal adverse trends in cell counts. CPK shows some variability at 15 mg and 30 mg with large error bars.

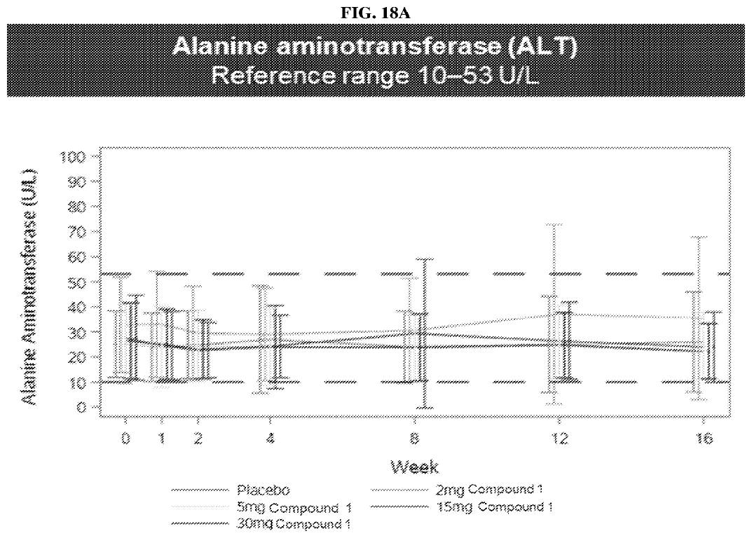

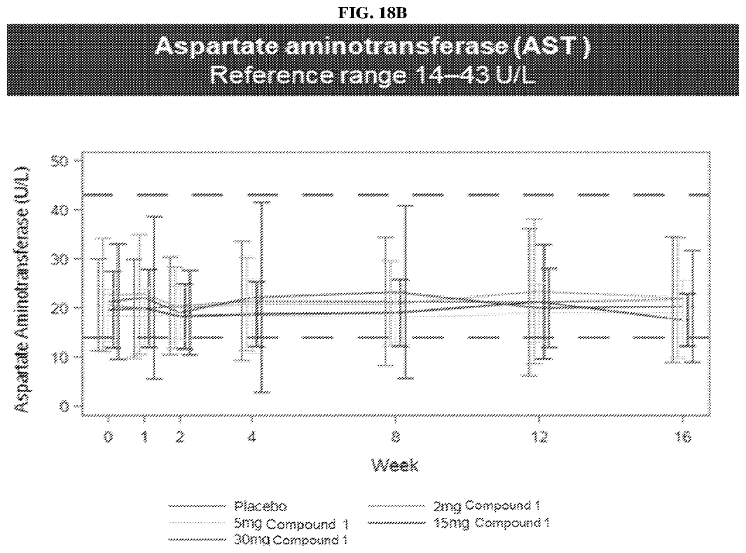

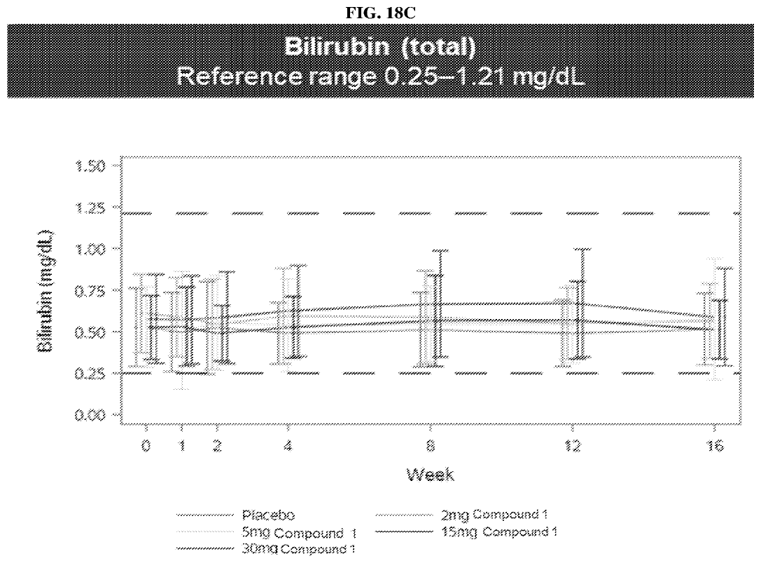

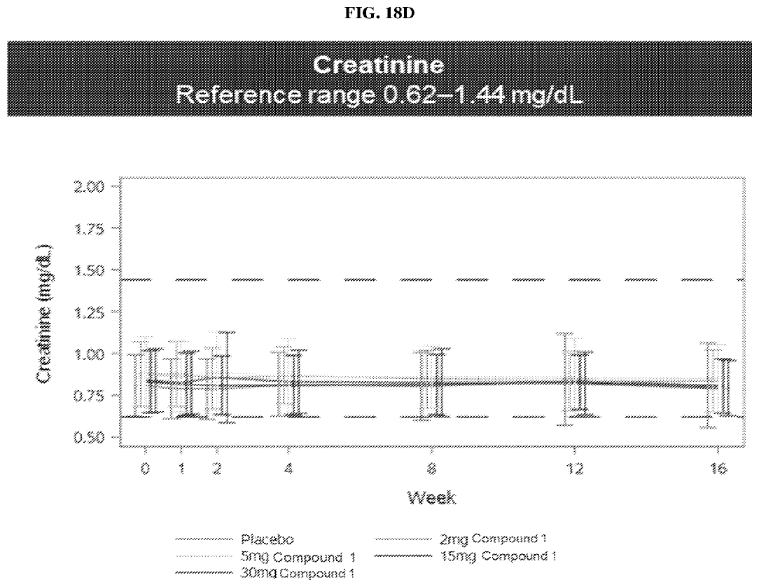

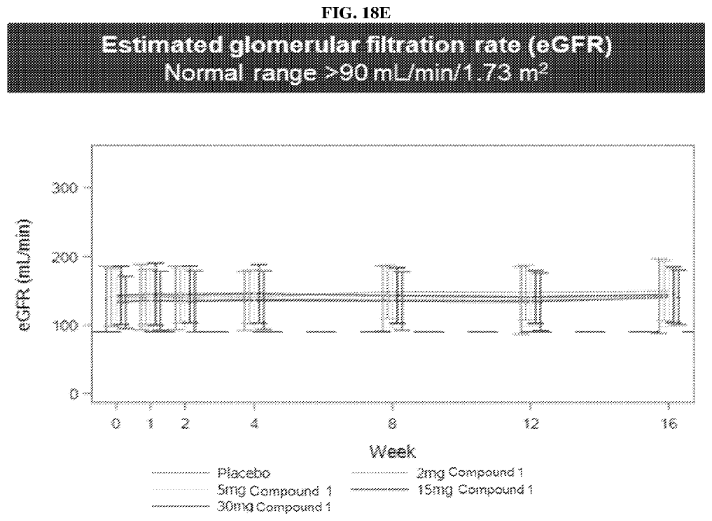

FIGS. 18A-18E show hepatic and renal parameters collected during the study. Data are mean±standard deviation. Mean lab values and changes from baseline do not reveal adverse trends for liver enzymes, creatinine or eGFR.

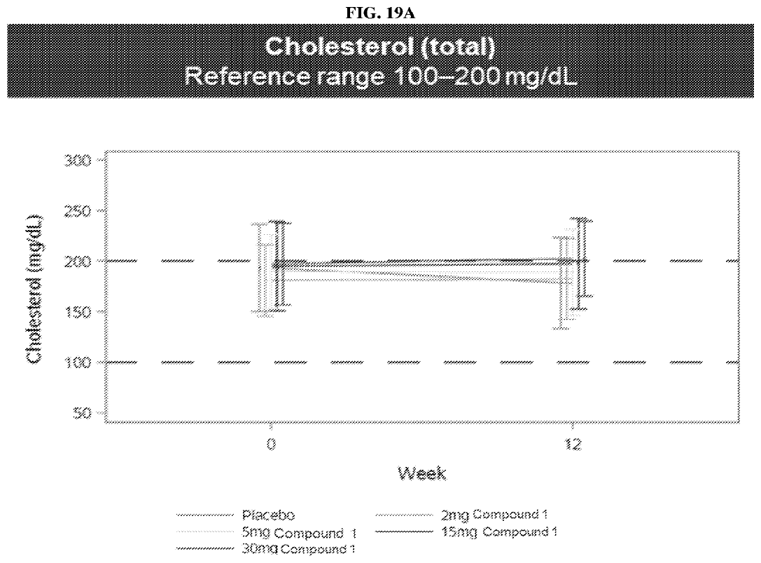

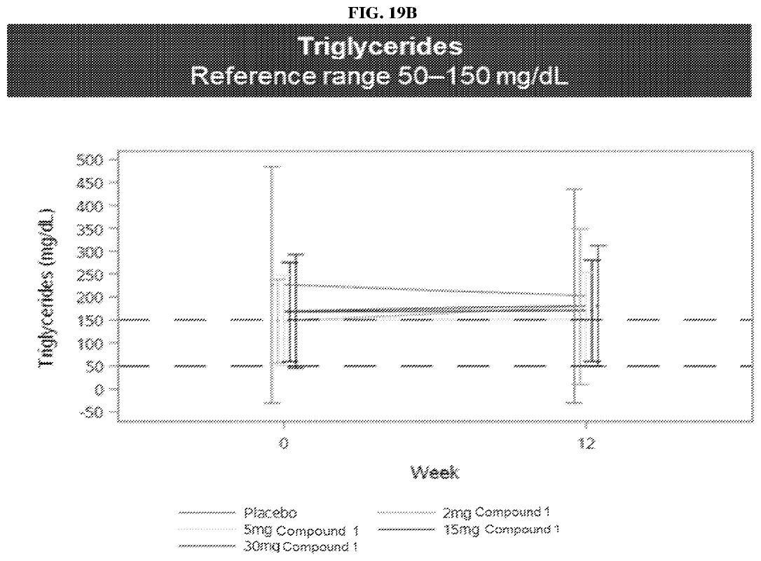

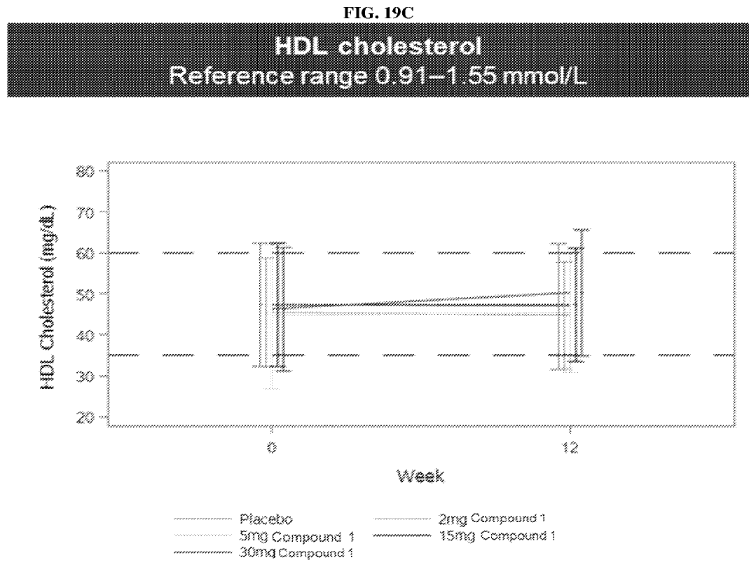

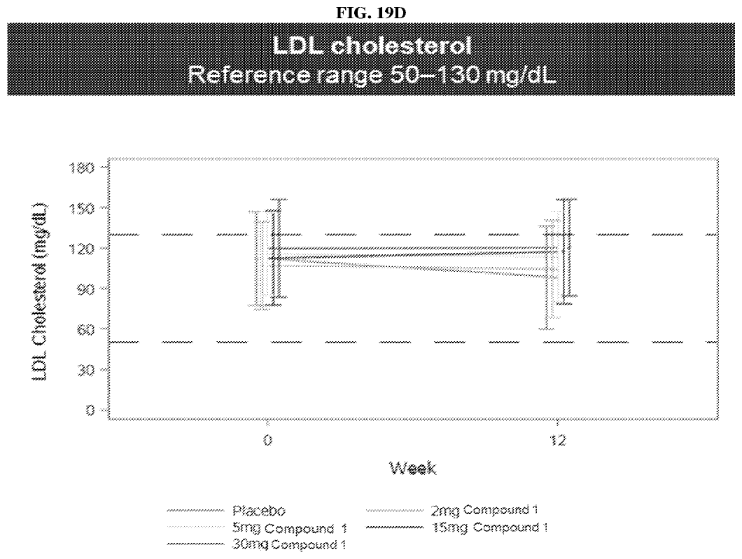

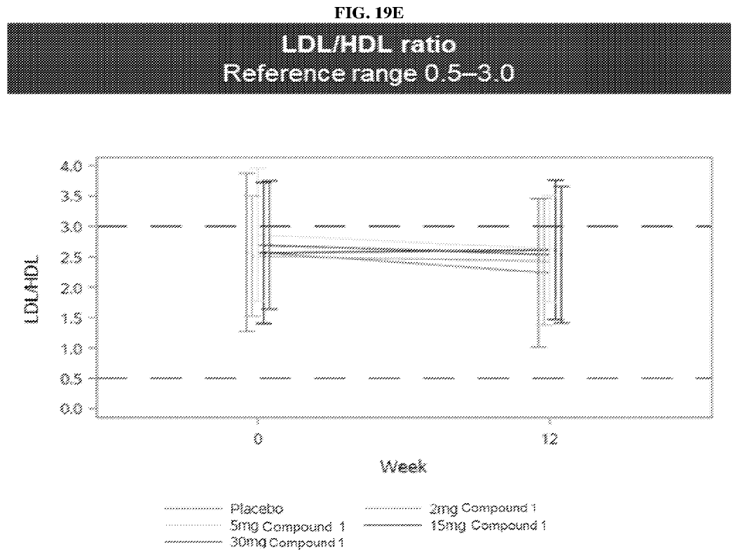

FIGS. 19A-19E show lipid parameters collected during the study. Data are mean±standard deviation. Mean lab values and changes from baseline do not reveal adverse trends for cholesterol, HDL, or LDL. Triglyceride elevation is minimal.

### Example 5: A Phase 2b, Randomized, Multi-Center, Double-Blind, Placebo-Controlled, Multiple-Dose Study to Evaluate the Efficacy, Safety, and Tolerability of Compound 1 in Subjects with Active Psoriatic Arthritis

### Study Sites:

Approximately 80 global study sites in North America and Europe will participate in this study.

### Number of Subjects (Planned):

Approximately 260 subjects will be randomized in this study (approximately 65 subjects/arm).

### Duration of Study:

The maximum study duration per subject is approximately 20 weeks, including up to 30 days for the screening period, a 12-week treatment period, and a 4-week safety follow-up period.

### Investigational Products, Dosage, and Mode of Administration:

Compound 1 at doses of 5 mg, 15 mg, 30 mg, or placebo will be orally administered once daily (QD) for 12 weeks. Compound 1 will be available in 5 mg and 15 mg strength capsules. Matching placebo will be identical to Compound 1 but will not contain the active ingredient.

Subjects will be randomized in a 1:1:1:1 ratio.

### Objectives:

The primary objective is:
To assess the efficacy of Compound 1 orally administered QD at 5 mg, 15 mg, or 30 mg for 12 weeks on the rheumatological signs, symptoms and function in subjects with active psoriatic arthritis (PsA).

The secondary objectives are:
To assess additional evaluations of efficacy of Compound 1 orally administered QD at 5 mg, 15 mg, or 30 mg for 12 weeks in subjects with active PsA
To assess the safety and tolerability of Compound 1 orally administered QD at 5 mg, 15 mg, or 30 mg for 12 weeks in subjects with active PsA
To evaluate the plasma concentration of Compound 1 orally administered QD at 5 mg, 15 mg, or 30 mg in subjects with active PsA

The exploratory objective is:
To evaluate the effects of Compound 1 on cytokines and other inflammatory biomarkers in blood in subjects with active PsA.

### Endpoints:

### Primary Efficacy Endpoint:

Proportion of subjects achieving at least an American College of Rheumatology (ACR) 20 response at Week 12

### Secondary Endpoints—Efficacy:

Proportion of subjects achieving at least an ACR 50 or ACR 70 response at Week 12
Change from baseline (Day 1) in tender joint count at Week 12
Change from baseline (Day 1) in swollen joint count at Week 12
Change from baseline (Day 1) in Patient Global Assessment of Psoriatic Arthritis at Week 12
Change from baseline (Day 1) in Patient Global Assessment of Psoriatic Arthritis Pain at Week 12
Change from baseline (Day 1) in Physician Global Assessment of Psoriatic Arthritis at Week 12
Change from baseline (Day 1) in Health Assessment Questionnaire-Disability Index (HAQ-DI) score at Week 12
Change from baseline (Day 1) in dactylitis count at Week 12, among subjects who have dactylitis at Day 1
Change from baseline (Day 1) in Leed's Enthesitis Index at Week 12, among subjects who have enthesitis at Day 1
Proportion of subjects with Minimal Disease Activity at Week 12
Change from baseline (Day 1) in Disease Activity Index for Psoriatic Arthritis at Week 12
Proportion of subjects achieving Psoriasis Area Severity Index (PASI)-75 at Week 12 among subjects with ≥3% body surface area (BSA) psoriatic involvement at Day 1
Proportion of subjects achieving a static Physician Global Assessment of 0 or 1 and at least a 2-point improvement from baseline at Week 12

### Secondary Endpoints—Safety:

Incidence of adverse events (AEs), treatment-emergent AEs (TEAEs), treatment-emergent serious adverse events (TESAEs) and TESAEs leading to discontinuation of study drug
Assessment of clinically relevant changes in vital signs, clinical laboratory parameters, proportion of subjects with clinically significant abnormal electrocardiograms (ECGs), and physical examinations
   Secondary endpoint—Pharmacokinetics:
Measurement of plasma concentrations of Compound 1 in subjects receiving 5 mg, 15 mg, or 30 mg of Compound 1

### Exploratory Endpoints:

Quantification of circulating cytokines and other inflammatory biomarkers
Change from baseline (Day 1) in 36-Item Short Form Health Survey Week 12
Change from baseline (Day 1) in Functional Assessment of Chronic Illness—Fatigue at Week 12
Change from baseline (Day 1) in Disease Activity Score 28 with high-sensitivity C-Reactive Protein (hsCRP) at Week 12
Change from baseline (Day 1) in Psoriatic Disease Activity Score at Week 12
Change from baseline (Day 1) in Psoriatic Arthritis Response Criteria at Week 12
Percent change from baseline (Day 1) in PASI at Week 12 among subjects with ≥3% BSA psoriatic involvement at Day 1
Change from baseline (Day 1) in BSA with psoriatic involvement at Week 12 among subjects with ≥3% BSA psoriatic involvement at Day 1
Change from baseline (Day 1) in Physician Global Assessment of Psoriasis at Week 12

### Study Design

This is a Phase 2b, randomized, multi-center, double-blind, placebo-controlled, multiple-dose study designed to evaluate the efficacy, safety, and tolerability of Compound 1 in subjects with active PsA. The evaluation of plasma concentrations of Compound 1 and exploration of pharmacodynamic changes in the levels of inflammatory cytokines in blood in subjects with active PsA treated with Compound 1 will also be performed.

Approximately 260 male and female subjects, aged 18 to 70 years, inclusive, with active PsA will be randomized in this study. To be eligible for the study, the subjects must have a history of PsA diagnosis with symptoms for ≥6 months prior to the screening visit and must meet all of the inclusion criteria, including the Classification Criteria for Psoriatic Arthritis (CASPAR) criteria, with ≥3 tender and ≥3 swollen joints at screening and baseline (Day 1) visits, and with active PsA despite previous therapy with non-steroidal anti-inflammatory drugs (NSAIDs), traditional disease-modifying anti-rheumatic drugs (DMARDs), or one tumor necrosis factor inhibitor (TNFi).

All subjects will read and sign a written informed consent form (ICF) prior to performing any screening procedures. Subjects who fulfill all of the inclusion criteria and none of the exclusion criteria will be included in the study. During a screening period of no longer than 30 days, subjects will be randomized (on Day −7) to receive either one of three doses of Compound 1 (5 mg, 15 mg, or 30 mg), or placebo on Day 1. The goal is to have approximately 65 subjects randomized per treatment group (1:1:1:1 ratio). During the treatment period, Compound 1 (5 mg, 15 mg, or 30 mg) or placebo will be orally administered QD for 12 weeks. The 12-week treatment period will be followed by a 4-week safety follow-up period.

For scheduled study visits, subjects will come to the study site on 8 occasions: screening, Day 1, and Weeks 1, 2, 4, 8, 12 (end of treatment/early termination visit), and 16 (end of study [EOS]).

Efficacy will be assessed using the ACR20 composite measure (including tender and swollen joint count, subject assessment of PsA pain visual analog scale [VAS], subject global PsA assessment VAS, physician global assessment PsA VAS, HAQ-DI, and hsCRP) as well as the individual components. Efficacy for psoriasis among subjects who have ≥3% BSA involvement on Day 1 will be measured using PASI, BSA, and Physician Global Assessment.

Safety will be assessed by collecting AEs, recording vital signs, performing physical examinations, and evaluating clinical laboratory and ECGs results.

Blood samples will be collected to measure plasma concentrations of Compound 1.

Blood samples will also be collected to evaluate the effect of Compound 1 on circulating inflammatory biomarkers.

On Day 1 (Visit 2) prior to dosing and 1 hour (±5 min) postdosing

Week 4 (Visit 5) prior to dosing, 1 hour (±5 min) postdosing, and 4 hours (t 10 min) postdosing

Week 8 (Visit 6) prior to dosing

Week 12 (Visit 7) anytime as study-drug dosing is completed, or at ET, whenever possible

Blood samples will also be collected on Day 1, and at Weeks 4 and 12, or at ET whenever possible, to evaluate the effect of Compound 1 on circulating inflammatory biomarkers.

No interim analysis is planned in this study.

### Inclusion Criteria:

In order to be eligible to participate in this study, a subject must meet all of the following criteria, either at the screening and Day 1 visits or only at one of the specified visits (screening or Day 1) as noted in the criteria:
1. Subject is male or female, aged 18 to 70 years, inclusive, at the time of consent.
2. Subject has PsA on the basis of the CASPAR with peripheral symptoms at the screening visit, as assessed by the investigator.
3. Subject has PsA symptoms for ≥6 months prior to screening, as assessed by the investigator.
4. Subject has ≥3 tender joints and ≥3 swollen joints at screening and Day 1 visits, as assessed by the investigator.
5. Subject has at least one lesion of plaque psoriasis ≥2 cm in diameter, nail changes characteristic of psoriasis, or a documented history of plaque psoriasis.
6. Subject has active PsA despite previous standard doses of NSAIDs administered for ≥4 weeks, or traditional DMARDs (including methotrexate and sulfasalazine) administered for ≥3 months, or TNFi agents administered for ≥3 months, or subjects are intolerant to NSAIDs or DMARDs or TNFi agents, as assessed by the investigator.
7. If subject is on concurrent PsA treatments, they must be on stable doses as described below and for the duration of the study:
   a. Methotrexate (MTX): subject must have received treatment for ≥3 months, with stable dose and stable route of administration (not to exceed 25 mg MTX per week) for ≥4 weeks prior to Day 1 until Week 16 (EOS); subjects on MTX should be taking folic acid supplementation according to local standard of care to minimize the likelihood of MTX associated toxicity.
   b. Sulfasalazine: Maximum dose of 3 gm/day. Minimum duration of therapy 2 months and dose stable for 4 weeks prior to Day 1.
   c. Other traditional DMARDs not listed may be considered on a case-by-case basis after discussion with the medical monitor.
   d. Oral corticosteroids: the subject must be on a stable dose, not to exceed the equivalent of 10 mg of prednisone per day, for ≥2 weeks prior to Day 1. If subject is not currently using oral corticosteroids, must not have received for at least 2 weeks prior to Day 1.
   e. NSAIDs or paracetamol/acetaminophen as needed: the subject must be on a stable dose for ≥2 weeks prior to Day 1. If not currently using NSAIDs, must not have received for at least 2 weeks prior to Day 1.
8. For female subjects of childbearing potential involved in any sexual intercourse that could lead to pregnancy: the subject must agree to use a highly effective contraceptive method from screening until at least 4 weeks after the last study drug administration. Highly effective contraceptive methods include hormonal contraceptives (e.g., combined oral contraceptive, patch, vaginal ring, injectable, or implant), intrauterine devices or intrauterine systems, vasectomized partner(s) (provided vasectomy was performed ≥4 months prior to screening), tubal ligation, or double barrier methods of contraception (e.g., male condom with cervical cap, male condom with diaphragm, and male condom with contraceptive sponge) in conjunction with spermicide. Note: Subjects must have been on a stable dose of hormonal contraceptives for at least 4 weeks before Day 1. Note: The above list of contraceptive methods does not apply to subjects who are abstinent for at least 4 weeks before Day 1 and will continue to be abstinent from penile-vaginal intercourse throughout the study. The reliability of sexual abstinence needs to be evaluated in relation to the duration of the clinical study, and the preferred and usual lifestyle of the subject. Periodic abstinence (calendar, symptothermal, postovulation methods) is not acceptable. Note: A female subject of nonchildbearing potential is defined as follows:
   a. Female subject who has had surgical sterilization (hysterectomy, bilateral oophorectomy, or bilateral salpingectomy)
   b. Female subject who has had a cessation of menses for at least 12 months prior to the screening visit without an alternative medical cause, and a follicle-stimulating hormone test confirming nonchildbearing potential (refer to laboratory reference ranges for confirmatory levels).
9. Female subjects of childbearing potential must have had a negative serum pregnancy test at screening and negative urine pregnancy test at Day 1.
10. For male subjects involved in any sexual intercourse that could lead to pregnancy, subject must agree to use one of the highly effective contraceptive methods listed in Inclusion Criterion 9, from Day 1 until at least 12 weeks after the last study drug administration. If the female partner of a male subject uses any of the hormonal contraceptive methods listed above, this contraceptive method should be used by the female partner from at least 4 weeks before Day 1 until at least 12 weeks after the last study drug administration. Note: Male subjects must refrain from donating sperm from Day 1 until at least 12 weeks after the last study drug administration. Note: No restrictions are required for a male subject who underwent a vasectomy at least 4 months prior to screening and the procedure is documented. If vasectomy procedure is not documented or was performed less than 4 months prior to screening, male subjects must follow the same contraception and sperm donation requirements as for nonvasectomized subjects.
11. Subject has a body mass index (BMI) of >18 kg/m2, inclusive, (BMI=weight (kg)/[height (m)]2), and total body weight >50 kg (110 lb).
12. Subject is willing to participate and is capable of giving informed consent. Note: Signed, dated, written informed consent must be obtained prior to any study-related procedures.
13. Subjects must be willing to comply with all study procedures and must be available for the duration of the study.

### Exclusion Criteria:

A subject who meets any of the following criteria at the screening and/or Day 1 visits, as applicable, will be excluded from participation in this study:
1. Subject has other disease(s) that might confound the evaluations of benefit of Compound 1 therapy, including but not limited to rheumatoid arthritis, axial spondyloarthritis (this does not include a primary diagnosis of PsA with spondylitis), systemic lupus erythematosus, Lyme disease or fibromyalgia.
2. Subject has a history of lack of response to any therapeutic agent targeting interleukin (IL)-12, IL-17, and/or IL-23 (e.g., ustekinumab, secukinumab, ixekizumab, brodalumab, guselkumab, tildrakizumab, risankizumab) at approved doses after at least 12 weeks of therapy, and/or received one of these therapies within 6 months prior to baseline (Day 1).
3. Subject has a history of lack of response to >1 therapeutic agent targeting tumor necrosis factor.
4. Subject has received infliximab, golimumab, adalimumab, or certolizumab pegol, or any biosimilar of these agents, within 8 weeks prior to baseline (Day 1).
5. Subject has received etanercept, or any biosimilar of etanercept, within 4 weeks prior to baseline (Day 1).
6. Subject has received rituximab or any immune-cell-depleting therapy within 6 months prior to baseline (Day 1).
7. Subject has received any marketed or investigational biological agent, other than those specified in other inclusion/exclusion criteria, within 12 weeks or 5 half-lives (whichever is longer) prior to baseline (Day 1).
8. Subject is currently receiving a non-biological investigational product or device or has received one within 4 weeks prior to baseline (Day 1).
9. Subject has received apremilast or other non-biologic systemic treatment for PsA within 4 weeks prior to baseline (Day 1), other than MTX, sulfasalazine, corticosteroids, NSAIDs, or paracetamol/acetaminophen, which are allowed at stable doses as described in Inclusion Criterion 7. Subject has received leflunomide within 8 weeks of baseline (Day 1) if no elimination procedure was followed or adhere to an elimination procedure (e.g., 11 days with cholestyramine or 30 days washout with activated charcoal as per local label). For subjects not receiving MTX and sulfasalazine at Screening, MTX and sulfasalazine are excluded within 4 weeks prior to baseline (Day 1).
10. Subject has received intraarticular injection (including corticosteroids), intramuscular steroids, intralesional steroids, or intravenous steroids within 4 weeks prior to baseline (Day 1). For subjects not receiving MTX and sulfasalazine at screening, MTX and sulfasalazine are excluded within 4 weeks prior to baseline (Day 1).
11. Subject has received high potency opioid analgesics (e.g., methadone, hydromorphone, or morphine) within 2 weeks prior to baseline (Day 1).
12. Subject has used any topical medication that could affect PsA or psoriasis (including corticosteroids, retinoids, vitamin D analogues [such as calcipotriol], Janus kinase [JAK] inhibitors, or tar) within 2 weeks prior to baseline (Day 1).
13. Subject has used any systemic treatment that could affect PsA or psoriasis (including oral retinoids, immunosuppressive/immunomodulating medication, cyclosporine, oral JAK inhibitors, or apremilast) within 4 weeks prior to baseline (Day 1), unless otherwise excluded or allowed by protocol. Note: Intranasal corticosteroids and inhaled corticosteroids are allowed. Eye and ear drops containing corticosteroids are also allowed.
14. Subject has received any ultraviolet (UV)—B phototherapy (including tanning beds) or excimer laser within 4 weeks prior to baseline (Day 1).
15. Subject has had psoralen and ultraviolet A (PUVA) treatment within 4 weeks prior to baseline (Day 1).
16. Subject has received Chinese traditional medicine within 4 weeks prior to baseline (Day 1).
17. Subject has received any live-attenuated vaccine, including for Coronavirus Disease-19 (COVID-19), within 4 weeks prior to baseline (Day 1) or plans to receive a live-attenuated vaccine during the study and up to 4 weeks or 5 half-lives of the study drug, whichever is longer, after the last study drug administration. Note: Non-live-attenuated vaccines for COVID-19 (e.g., RNA-based vaccines, inactivated adenovirus-based vaccines, protein-based vaccines) are allowed during the study. The study site should follow local guidelines related to COVID-19.
18. Subject is currently being treated with strong or moderate cytochrome P450 3A (CYP3A4) inhibitors, such as itraconazole or has received moderate or strong CYP3A4 inhibitors within 4 weeks prior to baseline (Day 1).
19. Subject has consumed grapefruit or grapefruit juice within 1 week prior to baseline (Day 1). Note: Consumption of grapefruit must be avoided during the treatment period and for at least 1 week after last study drug administration.
20. Subject has used tanning booths within 4 weeks prior to baseline (Day 1), has had excessive sun exposure, or is not willing to minimize natural and artificial sunlight exposure during the study. Note: Use of sunscreen products and protective apparel are recommended when sun exposure cannot be avoided.
21. Subject is a female who is breastfeeding, pregnant, or who is planning to become pregnant during the study.
22. Subject has evidence of erythrodermic, pustular, predominantly guttate psoriasis, or drug-induced psoriasis.
23. Subject has a history of skin disease or presence of skin condition that, in the opinion of the investigator, would interfere with the study assessments.
24. Subject has any clinically significant medical condition, evidence of an unstable clinical condition (e.g., cardiovascular, renal, hepatic, hematologic, gastrointestinal, endocrine, pulmonary, immunologic, or local active infection/infectious illness), psychiatric condition, or vital signs/physical/laboratory/ECG abnormality that would, in the opinion of the investigator, put the subject at undue risk or interfere with interpretation of study results.
25. Subject had a major surgery within 8 weeks prior to baseline (Day 1 or has a major surgery planned during the study.
26. Subject has a history of Class III or IV congestive heart failure as defined by New York Heart Association Criteria.
27. Subject has an estimated creatinine clearance of <40 mL/min based on the Cockcroft-Gault equation or a history of renal failure as defined by the investigator
   Cockcroft Gault equation:

$$Creatinine Clearance ( estimated ) / Conventional mL / min = ( 140 - Age [ years ] ) \times Weight ( kg ) \times Factor a / ( 72 \times serum creatinine [ mg / dL ] )$$

$$Note : Factor a = 0.85 in females and 1. in males .$$

28. Subject was hospitalized in the 3 months prior to screening for asthma, has ever required intubation for treatment of asthma, currently require oral corticosteroids for the treatment of asthma, or has required more than one short-term (≥2 weeks) course of oral corticosteroids for asthma within 6 months prior to baseline (Day 1).
29. Subject has a history of cancer or lymphoproliferative disease within 5 years prior to baseline (Day 1). Subjects with successfully treated nonmetastatic cutaneous squamous cell or basal cell carcinoma and/or localized carcinoma in situ of the cervix are not to be excluded.
30. Subject has a history of fever, inflammation, or systemic signs of illness suggestive of systemic or invasive infection within 4 weeks prior to baseline (Day 1).
31. Subject has an active bacterial, viral, fungal, mycobacterial infection, or other infection (including tuberculosis [TB] or atypical mycobacterial disease), or any major episode of infection that required hospitalization or treatment with intravenous antibiotics within 12 weeks prior to baseline (Day 1), or oral antibiotics within 4 weeks prior to baseline (Day 1).
32. Subject has a history of chronic or recurrent infectious disease, including but not limited to chronic renal infection, chronic chest infection, recurrent urinary tract infection, fungal infection (with the exception of superficial fungal infection of the nailbed), or infected skin wounds or ulcers.
33. Subject has a history of an infected joint prosthesis or has received antibiotics for a suspected infection of a joint prosthesis, if that prosthesis has not been removed or replaced.
34. Subject has active herpes infection, including herpes simplex 1 and 2 and herpes zoster (demonstrated on physical examination and/or medical history) within 8 weeks prior to Day 1.
35. Subject has a history of known or suspected congenital or acquired immunodeficiency state or condition that would compromise the subject's immune status in the opinion of the investigator (e.g., history of splenectomy, primary immunodeficiency).
36. Subject has positive results for hepatitis B surface antigens, antibodies to hepatitis B core antigens, hepatitis C virus (HCV), or human immunodeficiency virus. Samples testing positive for HCV antibodies will require polymerase chain reaction (PCR) qualitative testing for HCV RNA. Any HCV RNA PCR result that meets or exceeds detection sensitivity is exclusionary.
37. Subject has clinical or laboratory evidence of active or latent TB infection at screening as assessed by QuantiFERON-TB Gold (or a purified protein derivative [PPD] skin test, or equivalent, (or both if required per local guidelines) and chest X-ray. The PPD skin test should be utilized only when a QuantiFERON-TB Gold Test is not possible for any reason (unless local guidelines require both tests). Chest X-ray may be taken at screening or completed within 3 months prior to the screening visit, with documentation showing no evidence of infection or malignancy as read by a qualified physician.
   Note: Subjects with an history of active or latent TB will not be included in the study, unless documentation of prior and complete anti-TB treatment, appropriate in duration and type according to current local country guidelines, can be provided.
38. Subject with any of the following laboratory values at the screening visit:

$$a . Alanine aminotransferase or$$

$$aspartate aminotransferase values \geq 3 times the upper limit of normal ( ULN )$$

$$b . Hemoglobin < 11. g / dL ( < 110. g / L )$$

$$c . White blood cell count < 3.5 \times {10}^{9} / L ( < 3500 / {mm}^{3} )$$

$$d . Absolute neutrophil count of < 1.8 \times {10}^{9} / L ( < 1 \; 800 / {mm}^{3} )$$

$$e . Absolute lymphocyte count < 1. \times {10}^{9} / L ( < 1000 / {mm}^{3} )$$

$$f . Platelet count < 100 \times {10}^{9} / L ( < 100 , 000 / {mm}^{3} )$$

$$g . Total bilirubin > 2 times the ULN$$

39. Subjects who have given >50 ml of blood or plasma within 30 days of screening or >500 mL of blood or plasma within 56 days of screening (during a clinical study or at a blood bank donation).
40. Subject has a known or suspected allergy to Compound 1 or any component of the investigational product, or any other significant drug allergy (such as anaphylaxis or hepatotoxicity).
41. Subject has a known history of clinically significant drug or alcohol abuse in the last year prior to baseline (Day 1).

### Statistical Methods:

Categorical variables will be presented in tables as frequencies and percentages. Continuous variables will be summarized in tables including the number of subjects, mean, standard deviation, median, minimum, and maximum.

Further details regarding the efficacy and safety variable definitions, analyses strategy, statistical justification, and techniques for handling missing values (if applicable) will be detailed in a separate statistical analysis plan (SAP) that will be prepared before the database is locked and any analyses are undertaken. Any deviation(s) from the SAP will be described and justified in the final Clinical Study Report, as appropriate.

All statistical tests will be two-sided and will be performed with a significant level of 0.05, unless otherwise specified in the SAP. No adjustment to alpha will be made to account for multiple testing between treatment groups.

### Study Drug Exposure and Compliance:

Exposure to study drug and study drug compliance will be summarized for each treatment group for the Safety Analysis Set.

### Efficacy Analyses:

The primary endpoint can be translated as a responder analysis, where a subject will be classified as responder if they achieve ACR20 at Week 12. Comparison of the primary endpoint will be made between each dose group and the placebo group using the Cochran-Mantel-Haenszel (CMH) test adjusting for the randomization stratification factor (prior treatment with biologics and non-traditional DMARDs [yes/no] and region). The primary efficacy analysis will be performed on the full analysis set (FAS), while the intent-to-treat and per protocol sets will be used as a supplementary analysis. Subjects who prematurely discontinue study drug or who have missing data for the primary endpoint will be considered as non-responders for the primary analysis. Additional sensitivity analyses will be described in the SAP.

The primary efficacy analysis will also be performed in demographic subgroups including age, gender, race, and BMI to assess the consistency of the treatment effect. Additional subgroup analyses based on baseline disease characteristics and the randomization stratification factor will also be conducted and described in the SAP.

The secondary endpoints involving proportions of subjects will be analyzed using the same approach (CMH test) as described for the primary efficacy analysis and based on the FAS.

The continuous secondary endpoints involving change from baseline will be analyzed using a mixed model for repeated measures based on the FAS. The model will include fixed effects for treatment group, visit, and treatment group-by-visit interaction, with baseline score and the randomization stratification factor as covariates, and the change from baseline as the dependent variable. Additional details on sensitivity analyses and missing data imputation of the secondary endpoints will be provided in the SAP.

### Safety Analyses:

All safety analyses will be performed on the Safety Analysis Set. No inferential statistics are planned for safety variables.

Adverse events will be coded using Medical Dictionary for Regulatory Activities version 24.0 or higher and summarized by system organ class (SOC) and preferred term (PT). Analysis and reporting for AEs will be based on TEAEs. A TEAE is defined as an AE occurring (onset date/time) at the time of or after dosing on Day 1. Adverse events with missing start and/or end dates and/or times (if applicable) will be handled as described in the SAP.

All TEAEs will be listed for each subject. Separate listings will be presented for TEAEs leading to discontinuation of study drug and treatment-emergent serious adverse events (TESAEs). Any AEs occurring between signing of ICF and dosing on Day 1 will be presented in a separate listing. All listings will be done by treatment group and subject, detailing verbatim, SOC, PT, start date, stop date (if resolved), intensity, seriousness, outcome, action taken with respect to study drug, and relationship to study drug. The AE onset will also be shown relative (in number of days) to the day of study drug administration.

An overview of all TEAEs will be presented by treatment group. The overview will include the following:
Number and percentage of subjects with TEAEs
Number and percentage of subjects with drug related TEAEs
Number and percentage of subjects with TEAEs leading to discontinuation of study drug
Number and percentage of subjects with TESAEs
Number and percentage of subjects with TESAEs leading to discontinuation of study drug
Number and percentage of subjects by intensity of TEAEs (Common Terminology Criteria for Adverse Events grades 1, 2, 3, 4 5)
Number and percentage of subjects with TEAEs leading to death
Number and percentage of subjects with adverse events of special interest (AESIs)

Summaries of TEAEs by treatment group, SOC, and PT will be presented for the following:
All TEAEs
TESAEs
TESAEs leading to discontinuation of study drug
Drug-related TEAEs
TEAEs by intensity
AESIs

Results from vital signs, laboratory analyses, ECGs, and physical examination will be listed by subject and timepoint. Descriptive summaries of vital signs, laboratory analyses, ECGs and physical examination will also be provided.

### Pharmacodynamic Analyses:

Analyses of blood biomarker levels will be described in a separate analysis plan.

### Exploratory Analyses:

The continuous exploratory endpoints involving change from baseline or percentage change from baseline will be analyzed using a mixed model for repeated measures based on the FAS. Additional details will be provided in the SAP.

### Other Analyses:

Descriptive summaries of subject disposition and baseline characteristics (including demographic data and medical and surgical history) will be presented by treatment group, and also listed. Protocol deviations will be listed and summarized by treatment and category.

Medications will be coded using the World Health Organization Drug Dictionary and listed by subject. A summary of concomitant medications by treatment group and medication class will also be tabulated.

### Pharmacokinetic Analyses:

Plasma concentration data will be listed per subject and summarized descriptively per dose for each scheduled sampling time point.

### Sample Size Consideration:

A sample size of 65 per treatment group across all sites will have 83% power using a two-sided CMH test for two proportions (assuming an equal distribution among strata and a common odds ratio), assuming a type I error rate of 0.05, a 55% response rate for each Compound 1 dose group as a proportion of ACR20 response, and a placebo ACR20 response rate of 30%. A total number of 260 subjects (65 per treatment group) are planned to be randomized into the study with a 1:1:1:1 allocation.

The sample size was calculated in nQuery 8.7 using a two-sample CMH test for two proportions.

### Introduction—Psoriatic Arthritis

Psoriatic arthritis (PsA) is a chronic, systemic inflammatory disease that manifests as peripheral arthritis, axial disease, dactylitis, enthesitis, and skin and nail lesions. There is an estimated prevalence of 0.3 to 1% of the general population with psoriatic arthritis, with approximately 50% of subjects experiencing bone erosions within 2 years. Psoriatic arthritis is present in up to 30% of psoriasis subjects with disability from irreversible joint damage and deformities in many subjects. In addition, PsA is also associated with increased mortality. In 85% of psoriasis patients, skin disease precedes joint disease. The exact etiology of psoriatic arthritis is unknown, and both environmental factors and genetic predisposition appear to play roles in the onset of the disease.

Psoriatic arthritis substantially impacts function, quality of life, and work productivity. If left untreated, the joint inflammation results in joint destruction and disability. The goals of treatment are to ameliorate the signs and symptoms of PsA, improve function, prevent structural damage, and enhance quality of life and productivity. The significant morbidities associated with PsA are typically treated with non-steroidal anti-inflammatory drugs (NSAIDs), traditional disease-modifying anti-rheumatic drugs (DMARDs), and/or biologics such as tumor necrosis factor inhibitors (TNFi) or interleukin (IL)-12/23 or IL-17 inhibitors. There are limited data supporting the use of traditional DMARDS for PsA and these may not prevent structural damage. The biologics require parenteral delivery which are inconvenient for subjects. In addition, despite these therapies, a low percentage of subjects achieve remission, many have variable efficacy regarding the multiple co-morbid manifestations of PsA or are intolerant to available therapies. Thus, there continues to be a significant unmet need for a safe and efficacious oral PsA therapy.

### Study Rationale

Compound 1 has the potential to add value to the treatment algorithm of PsA, particularly considering the lack of highly efficacious oral agents. A safe, well-tolerated, and highly efficacious oral therapy for PsA would provide an appealing treatment option for both subjects and physicians.

Without wishing to be bound by theory, it is believed that inhibition of TYK2 by Compound 1 is expected to impact PsA pathogenesis primarily through its effects on the IL-23/Th17/Th22 axis. In addition, safety and efficacy data in Phase 2 PsA study presented to date for a TYK2 inhibitor (BMS-986165 or deucravacitinib), which also inhibits TYK2 activity through allosteric binding of the JH2 domain, sets the clinical precedence for selectively targeting TYK2 activity in this disease. Specific inhibition of TYK2 is expected to be more efficacious than any current oral therapies and could be comparable to the efficacy observed for some biologic therapies currently in use.

Treatment for PsA has changed dramatically over the last decade, with traditional synthetic DMARDs, glucocorticoids, biologic therapies (i.e., tumor necrosis factor [TNF] and IL inhibitors), biosimilar and targeted synthetic DMARDs, and other new targeted agents. Given the early efficacy and safety profile of Compound 1 in psoriasis, and the evidence of effects of another TYK2 inhibitor in PsA, the potential for Compound 1 to be effective in PsA is high.

### Study Treatment Administered

This study involves a comparison of Compound 1 at 5 mg, 15 mg, or 30 mg orally administered QD with a placebo. Compound 1 will be available in 5 mg, and 15 mg strength capsules.

All study drugs will be administered orally daily, as assigned, for 12 weeks. On study days, the study drugs will be administered at the study site (if applicable). The date and time of drug administration will be collected by the study site during each visit and daily via a diary provided to the subject. The subject should be instructed to take the study drug at approximately the same time of the day. For Day 1 and Week 12 visits, subjects should have fasted for at least 8 hours before their visit and up to the time of the blood draw for fasting lipid panel.

### Study Drugs

| | Study Drugs | | | |
|---|---|---|---|---|
| Drug name | Compound 1 | Compound 1 | Compound 1 | Placebo |
| Dosage form | Capsule | Capsule | Capsule | Capsule |
| Unit dose | 5 mg | 15 mg | 15 mg | N/A |
| strength(s) | | | | |
| Dose level(s) | 5 mg | 15 mg | 30 mg | N/A |
| Number of | 1 active | 1 active | 2 active | 2 placebo |
| capsules per | 1 placebo | 1 placebo | | |
| dose level | | | | |
| Route of | Oral | Oral | Oral | Oral |
| administration | | | | |
| Dosing | QD with | QD with | QD with | QD with |
| instructions | approximately | approximately | approximately | approximately |
| | 240 mL of water | 240 mL of water | 240 mL of water | 240 mL of water |
| Physical | Opaque white | Opaque white | Opaque white | Placebo is |
| description | capsules | capsules | capsules | identical to the |
| | | | | active capsules in |
| | | | | size, shape, and |
| | | | | color. |

N/A = not applicable; QD = once daily.

### Permitted Therapies

The following therapies are permitted:

Intranasal corticosteroids and inhaled corticosteroids are allowed. Eye and ear drops containing corticosteroids are also allowed; and the use of oral corticosteroids and concomitant PsA treatments (i.e., MTX and sulfasalazine).

Use of sunscreen products and protective apparel are permitted when sun exposure cannot be avoided. However, on the day of scheduled visits, subjects cannot apply sunscreen products before their scheduled visit time.

### Emollients

Subjects can apply an emollient of their choice (except those containing salicylic acid) on their skin, including on psoriasis lesions. However, on the day of scheduled visits, subjects cannot apply emollient before their scheduled visit time.

Every effort should be made to keep the same emollient throughout the study for the same body region. However, the chosen emollient may differ depending on the body region (e.g., body vs face emollient may be different). The commercial name of the selected emollient(s) will be recorded in the source document and the eCRF. No other products may be applied to the lesions during the study.

### Prohibited Therapies or Procedures

Prohibited medications that are not to be used from the defined washout periods before the first administration of study drug at the Day 1 visit through the last study visit are provided in the table below.

Subjects who start prohibited medications (systemic and topical) or therapies that have been demonstrated to be effective for treatment of PsA or plaque psoriasis during the study may be discontinued from study drug. Investigators should contact the medical monitor as soon as possible when a prohibited medication is initiated, to discuss the appropriate steps. Subjects who start prohibited medications or therapies for other reasons during the study may be discontinued from study drug if an impact on efficacy assessment or safety of the subjects is expected.

### Prohibited Therapies or Procedures

| | Washout Period Prior To |
|---|---|
| Prohibited Medications, Products, And Procedures | First Dose (Day 1) |
| DMARD Therapies | |
| Therapeutic agent targeting IL-12, IL-17, and/or IL-23 (e.g., | 6 months |
| ustekinumab, secukinumab, ixekizumab, brodalumab, guselkumab, | |
| tildrakizumab, risankizumab) | |
| Rituximab or other immune-cell depleting agents | 6 months |
| Therapeutic agent targeting TNF-α (e.g., infliximab, golimumab, | 8 weeks |
| adalimumab, or certolizumab pegol) or any biosimilar of these agents | |
| Leflunomide if no elimination procedure was followed. Or if an | 8 weeks |
| elimination procedure is used then 11 days with cholestyramine or | |
| 30 days washout with activated charcoal as per local label. | |
| Apremilast or other non-biologic systemic treatment for PsA, other | 4 weeks |
| than MTX, sulfasalazine, corticosteroids, NSAIDs, or | |
| paracetamol/acetaminophen, which are allowed at stable doses as | |
| described in inclusion criterion | |
| Etanercept or any biosimilar of etanercept | 4 weeks |
| Investigational Drugs or Vaccines | |
| Any marketed or investigational biological agent (except those | 12 weeks or 5 half-lives |
| listed above) | (whichever is longer) |
| Any live-attenuated vaccine, including for COVID-19 or plans or receive | 4 weeks or 5 half-lives |
| a live-attenuated vaccine | (whichever is longer) |
| Note: Non-live-attenuated vaccines for COVID-19 (e.g., RNA-based | |
| vaccines, inactivated adenovirus-based vaccines, protein-based | |
| vaccines) are allowed during the study. The study site should follow | |
| local guidelines related to COVID-19. | |
| Non-biological investigational drug or device | 4 weeks |
| Steroids and Similar Therapies | |
| Systemic treatment that could affect psoriasis or PsA (oral retinoids, | 4 weeks |
| immunosuppressive/immunomodulating medication, cyclosporine, | |
| oral JAK inhibitors, or apremilast) | |
| Note: Intranasal corticosteroids and inhaled corticosteroids are allowed. | |
| Eye and ear drops containing corticosteroids are also allowed. | |
| Intraarticular injection (including corticosteroids), intramuscular steroids, | 4 weeks |
| intralesional steroids, or intravenous steroids | |
| Topical medication that could affect psoriasis (including corticosteroids, | 2 weeks |
| retinoids, vitamin D analogues [such as calcipotriol], JAK inhibitors, or | |
| tar) | |
| Antibiotics/Antifungals/CYP3A4 inhibitors | |

| | Washout Period Prior To | |
|---|---|---|
| Prohibited Medications, Products, And Procedures | First Dose (Day 1) | |
| Intravenous antibiotic treatment | 12 | weeks |
| Strong or moderate CYP3A4 inhibitors (such as itraconazole) | 4 | weeks |
| Oral antibiotic treatment | 4 | weeks |
| Grapefruit or grapefruit juice | 1 | week |
| Note: Consumption of grapefruit must be avoided during the treatment | | |
| period and for at least 1 week after last dose administration. | | |
| All Other Prohibited Medications/Therapy | | |
| PUVA treatment, UV-B phototherapy (including tanning beds), or | 4 | weeks |
| excimer laser, or tanning booths, has had excessive sun exposure, or is | | |
| not willing to minimize natural and artificial sunlight exposure during | | |
| the study | | |
| Note: Use of sunscreen products and protective apparel are recommended | | |
| when sun exposure cannot be avoided. | | |
| Chinese traditional medicine | 4 | weeks |
| High potency opioid analgesics (e.g., methadone, hydromorphone, | 2 | weeks |
| or morphine) | | |

COVID-19 = coronavirus Disease-2019; CYP3A4 = cytochrome P450 3A; IL = interleukin; JAK = Janus kinase; MTX = methotrexate; NSAIDs = nons-steroidal inflammatory drugs; PsA—psoriatic arthritis; PUVA = psoralen-ultraviolet-A; RNA = ribonucleic acid; TNF-α—tumor necrosis factor alpha; UV = ultraviolet.

### Efficacy Assessments

Clinical evaluations of PsA will be performed by an experienced and qualified physician (board certified or equivalent) or other suitably qualified and experienced designee. To assure consistency and reduce variability, the same assessor, who is not involved in the study conduct or direct patient care should perform all clinical efficacy assessments on a given subject whenever possible.

Detailed descriptions of the endpoints will be provided in a separate study manual.

### Primary Efficacy Endpoint—American College of Rheumatology 20

The ACR-20 will be assessed at each visit specified above. The ACR20 is a composite measure defined as both improvcmcnt of 20% in the number of tcndcr (68) and number of swollen (66) joints, and a 20% improvement in three of the following five criteria: patient global assessment of psoriatic arthritis, physician global assessment of psoriatic arthritis, patient pain scale, disability history questionnaire (i.e., HAQ-DI) and an acute phase reactant (i.e., erythrocyte sedimentation rate [ESR] or hsCRP). For this primary endpoint, hsCRP will be used.

### Secondary Efficacy Endpoints

### American College of Rheumatology 50/70

The ACR-50 and ACR-70 response will be assessed at each visit. The ACR-50 and ACR-70 are a composite measure defined as both improvement of 50% or 70%, respectively, in the number of tender (68) and number of swollen (66) joints, and a 50% or 70%, respectively, improvement in three of the following five criteria: patient global assessment of psoriatic arthritis, physician global assessment of psoriatic arthritis, patient pain scale, disability history questionnaire (i.e., HAQ-DI) and an acute phase reactant (i.e., ESR or CRP).

### Tender Joint Count 68 and Swollen Joint Count 66

The TJC 68 and SJC 66 (SJC minus hip joints, which cannot be assessed for swelling) are a total score of points assigned for presence of tenderness or swelling in the following:
Temporomandibular, sternoclavicular, acromioclavicular, shoulder, elbow, wrist, hip (TJC only), knee, ankle, tarsus, typically assigned 2 points each
Metacarpophalangeal, finger proximal interphalangeal, metatarsophalangeal, toe proximal interphalangeal, typically assigned 10 points each
Distal interphalangeal, typically assigned 8 points

### Patient Global Assessment of Psoriatic Arthritis

Subjects will rate their assessment of their PsA using a VAS where 0 is ‘very good, no symptoms’ and 100 is ‘very poor, severe symptoms’.

### Patient Global Assessment of Psoriatic Arthritis Pain

Subjects will rate their assessment of their PsA using a VAS where 0 is ‘no pain’ and 100 is ‘most severe pain’.

### Physician Global Assessment of Psoriatic Arthritis

The investigator or qualified sub-investigator will assess the patients' overall disease status, taking into account signs, symptoms, and function, of all components of joint and skin which is affected at the time of the visit and will rate this overall status using a VAS scale where 0 is ‘very good, asymptomatic, and no limitation of normal activities’ and 100 is ‘very poor, very severe symptoms which are intolerable, and inability to carry out all normal activities’.

### Health Assessment Questionnaire—Disability Index

The HAQ-DI will be assessed at the visits specified above. The HAQ-DI is comprised of eight domains: dressing, arising, eating, walking, hygiene, reach, grip, and activities. There are two or three questions per section. Scoring within each section is from 0 (without any difficulty) to 3 (unable to do). For each section the score given to that section is the worst score within the section (i.e., if one question is scored 1 and another 2, then the score for the section is 2). In addition, if an aide or device is used or if help is required from another individual, then the minimum score for that section is 2. If the section score is already 2 or more then no modification is made. The eight scores of the eight sections are summed and divided by eight. The result is the DI or disability index.

### Dactylitis Count

The dactylitis count consists of totaling the number of single digits in the hands and feet with tenderness.

Disease Activity Score 28 with CRP

The DAS28-CRP will be assessed at the visits specified above. The DAS28-CRP describes the severity of PsA using clinical and laboratory data, specifically hsCRP. The DAS scores indicate how active a subject's psoriatic arthritis is currently and can be trended over time.

### Leed's Enthesitis Index

The LEI will be assessed at the visits specified above. The LEI is comprised of review of six bilateral sites: Achilles tendon insertions, medial femoral condyles, and lateral epicondyles of the humerus. Tenderness at each site is quantified on a dichotomous basis: 0 means nontender and 1 means tender.

### Physician Global Assessment of Psoriasis

The PGA will be assessed at the visits specified above. The PGA is measured using a 0 to 4 scale with a 0 score meaning cleared and a 4 score meaning severe and a ≥2 grade improvement from baseline at a specified timepoint, specifically Week 12 in this study.

### Exploratory Efficacy Endpoints

### Disease Activity Index for Psoriatic Arthritis

The DAPSA will be assessed at the visits as specified above. The DAPSA score is a composite score calculated using scores from TJC of 68 joints and SJC of 66 joints, patient's global and pain scores on a VAS, and the CRP level. A DAPSA score of 5-14 represents a state of low disease activity and a score of <4 represents remission.

### Psoriatic Disease Activity Score

The PASDAS will be assessed at the visits as specified above. The PASDAS is a composite score which evaluates multiple aspects of PsA. It includes patient and physician global scores of skin and joint disease, swollen and tender joint counts, the LEI, tender dactylitis count, the physical component of the SF-36, and CRP level.

### Psoriatic Arthritis Response Criteria

The PsARC will be assessed at the visits as specified above. The PsARC is recommended in the assessment and monitoring of PsA. It consists of four components: assessment of TJC68 and SJC66, patient global assessment, and physician global assessment.

### Psoriasis Area and Severity Index

The PASI will be evaluated at the visits as specified above. This index quantifies the severity of a subject's psoriasis based on both lesion severity and the percentage of BSA affected. The PASI is a composite score ranging from 0 to 72 that that takes into account the degree of erythema, induration/infiltration, and desquamation (each scored from 0 to 4 separately) for each of four body regions, with adjustments for the percentage of BSA involved for each body region and for the proportion of the body region to the whole body.

### Body Surface Area

The overall BSA affected by plaque psoriasis will be evaluated (from 0% to 100%) at the visits specified above. The palmar surface of one hand (using the subject's hand and including the fingers) represents 1% of his or her total BSA. To be eligible, subjects must have a BSA affected by plaque psoriasis of at least 10% at screening and Day 1 visit.

### Minimal Disease Activity

Minimal Disease Activity measured as follows: the patient meets at least 5 of the 7 criteria. The criteria are: 1) tender joint count <1; 2) swollen joint count <1; 3) PASI score <1 or BSA <3%; 4) Patient Global Assessment of PsA Pain, VAS score <15; 5) Patient Global Assessment of PsA, VAS score <20; 6) HAQ-DI score <0.5; 7) tender entheseal points, using the LEI<1.

High sensitivity C-Reactive Protein

Hs—CRP will be measured in the central laboratory to help track inflammation over time. Moderately elevated CRP, in conjunction with other assessments of efficacy will be used together to determine certain efficacy endpoints.

### Erythrocyte Sedimentation Rate

The ESR will be measured at the local site to help indicate the level of inflammation in the body. The ESR, in conjunction with other assessments of efficacy will be used together to determine certain efficacy endpoints.

### Statistical Analyses

### General Considerations

Statistical analyses will include tabulations of summary data, inferential analyses, by-subject listings, and figures.

All statistical tests will be two-sided and will be performed at the 5% level of significance, unless otherwise stated. Frequency counts (n and percentages) will be made for each qualitative variable. Descriptive statistics (n, mean, SD, median, minimum, and maximum) will be calculated for each quantitative variable, unless otherwise stated. All data will be summarized by treatment group.

The baseline value for analysis and reporting will be based on the last (non-missing) measurement before dosing on Day 1. If any safety measurements are repeated after dosing on Day 1, then the first (non-missing) value of any repeated measurements will be used in the descriptive statistics and in the calculation of changes from baseline.

### Efficacy Analyses

The primary endpoint can be translated as a responder analysis, where a subject will be classified as responder if they achieve ACR20 at Week 12. Comparison of the primary endpoint will be made between each dose group and the placebo group using the Cochran-Mantel-Haenszel (CMH) test adjusting for the randomization stratification factor (prior treatment with biologics and non-traditional DMARDs [yes/no], and region). The primary efficacy analysis will be performed on the FAS, while the ITT and PP sets will be used as a supplementary analysis. Subjects who prematurely discontinue study drug or who have missing data for the primary endpoint will be considered as non-responders for the primary analysis. Additional sensitivity analyses will be described in the SAP.

The primary efficacy analysis will also be performed in demographic subgroups including age, gender, race, and body mass index to assess the consistency of the treatment effect. Additional subgroup analyses based on baseline disease characteristics and the randomization stratification factor will also be conducted and described in the SAP.

The secondary endpoints involving proportions of subjects will be analyzed using the same approach (CMH test) as described for the primary efficacy analysis and based on the FAS.

The continuous secondary endpoints involving change from baseline will be analyzed using a mixed model for repeated measures based on the FAS. The model will include fixed effects for treatment group, visit, and treatment group-by-visit interaction, with baseline score and the randomization stratification factor as covariates, and the change from baseline as the dependent variable. Additional details on sensitivity analyses and missing data imputation of the secondary endpoints will be provided in the SAP.

### Example 6: Treatment of IBD with Compound 1

Inflammatory Bowel Disease (IBD) represents one of the key potential differentiation opportunities for our selective allosteric TYK2 inhibitor, Compound 1, where we believe the selectivity and PK and PD profile of Compound 1 may enable higher targeted TYK2 inhibition while maintaining a favorable tolerability profile.

IBD is an inflammation of the gastrointestinal tract caused by an immune response to environmental triggers. According to the Decision Resources Group, the two most common forms of IBD, ulcerative colitis and Crohn's disease, are estimated to affect approximately 1.9 million adults in the U.S. Ulcerative colitis and Crohn's disease are distinguished primarily by the affected portion of the gastrointestinal tract. Ulcerative colitis is characterized by inflammation of the large intestine or colon and the rectum, while Crohn's disease encompasses inflammation of any part of the gastrointestinal tract, but most often impacts the end of the small intestine or the ileum where it joins the colon. We believe that approximately 980,000 adults in the U.S. and an equal number of adults in the EU4 and the UK are diagnosed with ulcerative colitis, and approximately 900,000 adults in each of the U.S. and the EU4 plus the UK are diagnosed with Crohn's disease.

The approach to IBD treatment is determined by multiple factors, including disease severity, location of inflammation, previous response to treatment, side effects and co-morbidities. The most common oral treatments for ulcerative colitis are anti-inflammatories that contain 5-aminosalicylic acid (5-ASA). These drugs decrease inflammation at the intestinal wall and may reduce symptoms and maintain remission in ulcerative colitis but are not as effective in treating Crohn's disease. Corticosteroids and other oral immunomodulators may be used for short-term control of flare-ups and to maintain remission in IBD patients who have not responded to other medications.

Injectable biologics generally are reserved for treatment of moderate-to-severe ulcerative colitis and Crohn's disease. The most commonly prescribed biologics are anti-TNFα biologics (including AbbVie's Humira®, Johnson & Johnson's Remicade® and Simponi®, and UCB Pharma's Cimzia®). Newer biologic treatment options include anti-integrin therapies for ulcerative colitis (Takeda's Entyvio®) and anti-IL-12/IL-23 antibodies (e.g., Johnson & Johnson's Stelara®) for Crohn's disease. Pfizer's oral JAK inhibitor, Xeljanz®, was approved for treatment of ulcerative colitis in 2018; however commercial uptake has been limited due to the aforementioned safety concerns. BMS's Zeposia®, an S1P1R oral modulator, was approved for treatment of moderate-to-severe ulcerative colitis in May 2021.

In 2020, the IBD market for all levels of severity was approximately $14 billion in the U.S. and $25 billion globally. Global reported sales of treatments for ulcerative colitis totaled an estimated $7.9 billion in 2020, while global annual sales of treatments for Crohn's disease totaled an estimated $17.7 billion in 2020. Market research suggests the IBD commercial market has significant growth potential driven by increasing disease incidence and the emergence of novel oral therapeutics. We believe projected gains for oral agents are supported by physician and patient preference for oral administration over injectable biological therapies, high demand for new therapies with competitive clinical profiles, and the potential for potent and well-tolerated oral agents to expand the overall treated moderate-to-severe ulcerative colitis population.

TNF inhibitor sales currently dominate the ulcerative colitis treatment market, closely followed by sales of other injectable biologic therapies (anti-IL-12/23 antibodies and anti-integrin therapies), and oral 5-ASA therapies.

### Example 7: Absorption, Distribution, Metabolism, Excretion (ADME) and Other Properties of Compound 1

The potency and selectivity for Compound 1 were explored by screening Compound 1 at a 1 micromolar concentration for binding against a panel of 631 kinases. Only one kinase was inhibited with >50% inhibition: PIP51K1C (54% inhibition). Various in vitro potency/selectivity results are summarized in the table below.

| In Vitro Potency/Selectivity and Other | | |
|---|---|---|
| Properties | Compound 1 | |
| ALog D | 2.0 | |
| Caco-2 (A-B Pₐₚₚ/Efflux Ratio) | 15/1 | |
| TYK2 JH2 Kd | 0.0038 | nM |
| TYK2 PBMC IL 12 pSTAT4 IC₅₀ | 6.7 | nM |
| hWB IFNα IP-10 IC₅₀ | 51 | nM |
| mWB IFNα IP-10 IC₅₀ | 347 | nM |
| rWB IFNα IP-10 IC₅₀ | 91 | nM |
| TYK2/JAK1-3 JH1 Kds | >30,000 | nM |
| JAK1 JH2 Kd | 5,000 | nM |
| JAK2 JH2 Kd | 23,000 | nM |
| JAK2 PBMC GMCSF pSTAT5 IC₅₀ | >50,000 | nM |
| JAK1/3 PBMC IL 2 pSTAT5 IC₅₀ | >50,000 | nM |
| PDE4D IC₅₀ | >10,000 | nM |
| hERG (Patch Clamp) IC₅₀ | >30,000 | nM |

| | |
|---|---|
| 87 target panel of enzymes, ion | <50% inhibition for 85 |
| channels, receptors @ 10,000 nM | targets; only two enzymes were |
| | inhibited with >50% inhibition: |
| | LCK (54%) and the adenosine |
| | transporter (53%) |

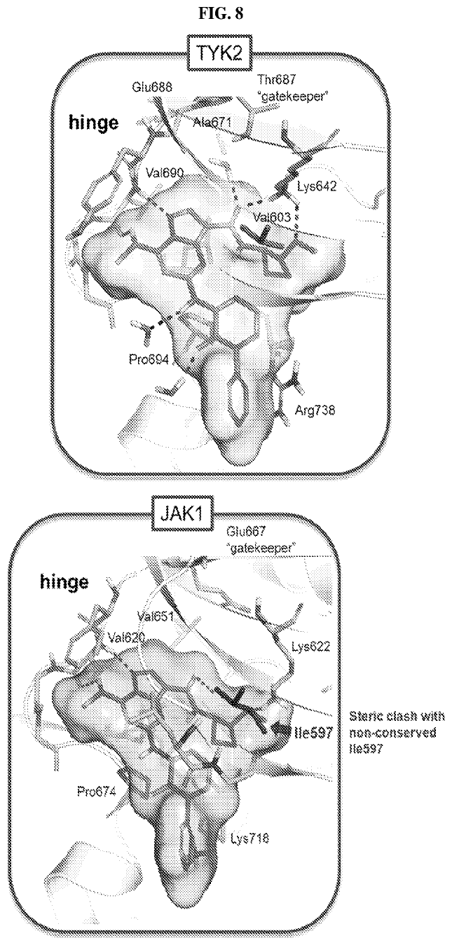

FIG. 8 shows X-ray crystallographic results of Compound 1 bound to TYK2 (top) and molecular docking studies of Compound 1 in JAK1 (bottom). These studies showed a clear structural basis for the excellent selectivity of Compound 1 for TYK2 binding vs. JAK1 binding. Compound 1 TYK2 JH2 domain K_{d} calculated was 0.0038 nm (X-ray resolution 1.83 Å), while the calculated KC_{d} of Compound 1 for the JAK1 J1H2 domain was 5,000 nM (modeled from PDB 4L00; see A. V. Toms, et al. Nat. Struct. Mol. Biol. 2013, DOI: 10.1038/nsmb.2673.). Modeling showed a steric clash between Compound 1 and a non-conserved Ile597 residue of JAK1 JH2; similar clash with Ile559 (from PDB 5UT6; see A. S. Newton, et at ACS Med. Chem. Lett. 2017, DOI: 10.1021/acsmedchemlett.7b00154.).

Drug metabolism and pharmacokinetic (PK) studies were conducted with Compound 1 in rodent (mouse and rat) and non-rodent (dog and monkey) species and systemic exposure to Compound 1 following oral administration was evaluated in the completed nonclinical safety studies.

| | In Vitro | | In Vivo | | | | | | |
|---|---|---|---|---|---|---|---|---|---|
| | Hep Cl_{pred} | PPB Fᵤ | Cl_{p, obs} | Vss | t_{1/2} | PO | | tₘₐₓ | |
| | (mL/min/kg) | (%) | (mL/min/kg) | (L/kg) | (h) | Dose | | (h) | % F |
| Mouse | 54 | 8 | 16 | 1.3 | 2.0 | 30 | mpk | 0.4 | 81 |
| Rat | 30 | 15 | 26 | 1.6 | 1.2 | 10 | mpk | 1.7 | 63 |
| Dog | 15 | 23 | 14 | 4.0 | 5.3 | 10 | mpk | 0.83 | 47 |
| Cyno | 11 | 23 | 7 | 1.9 | 3.8 | 5 | mpk | 3.0 | 47 |

| | | | | |
|---|---|---|---|---|
| Human | 0.69* | 23 | 1.7-7.5** | 1.2-10** |

Preclinical Cl and Vss from 1 mpk IV dose
*Hepatopac low turnover assay
**Predicted from Wajima allometric and PBPK-gut models

Compound 1 showed good absorption across species. The FaSSIF/FeSSIF Solubilities were 13/70 μg/mL, respectively (kinetic solubility 32 μM). Caco-2 Pₐₚₚ (A-B) was 15×10⁻⁶ cm/sec, efflux ratio=1. DDI perpetrator potential was low at clinically relevant exposures. CYP450 IC₅₀s: >30 μM at 1A2, 2C9, 2D6, >8 μM at 2C19 and 3A4 (M), 2 uM 3A4 (T), no time-dependent inhibition; low PXR activation (26% of rifampicin control at 30 μM). We observed low overall metabolism; human in vitro metabolites were observed in hepatocytes in preclinical species (rat, cynomolgus monkeys). Elimination was primarily via oxidative metabolism by multiple CYP450 enzymes followed by conjugation.

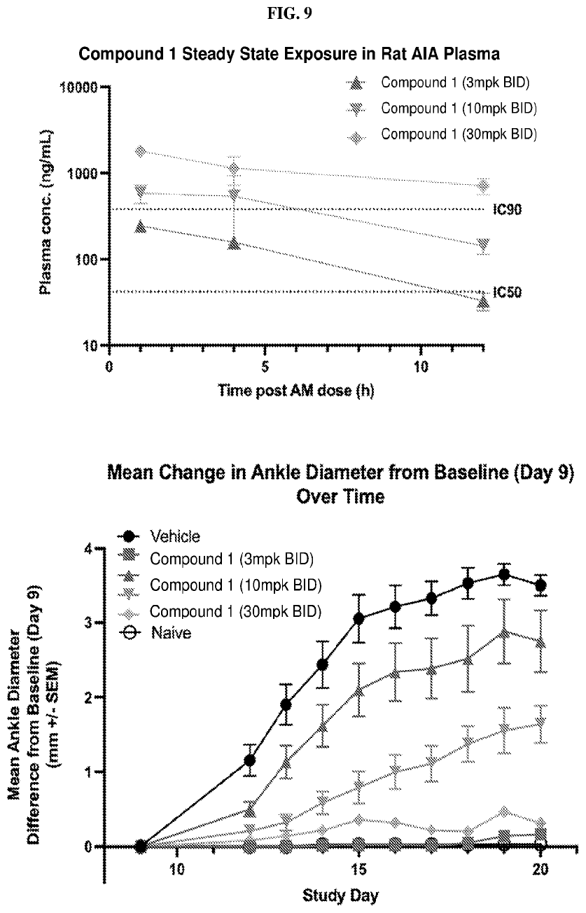

As shown in FIG. 9, Compound 1 suppressed disease activity in a dose-related manner in a rat adjuvant-induced arthritis (AIA) efficacy model. On day 0, the adjuvant was injected. On day 9, baseline ankle diameters were measured. On days 12-20, daily ankle diameter measurements were taken. On day 20, a final necroscopy and paw weight measure were taken. Compound 1 was administered daily on all 20 days. Compound 1 exposure was assessed on Day 19. IC_{50/90} reference lines (top panel) represent inhibitory concentrations in a rat whole-blood TYK2-dependent IFNα-induced IP10 assay. Concentration-dependent suppression of TYK2-dependent IL12-induced INFγ production also observed in rat (not shown).

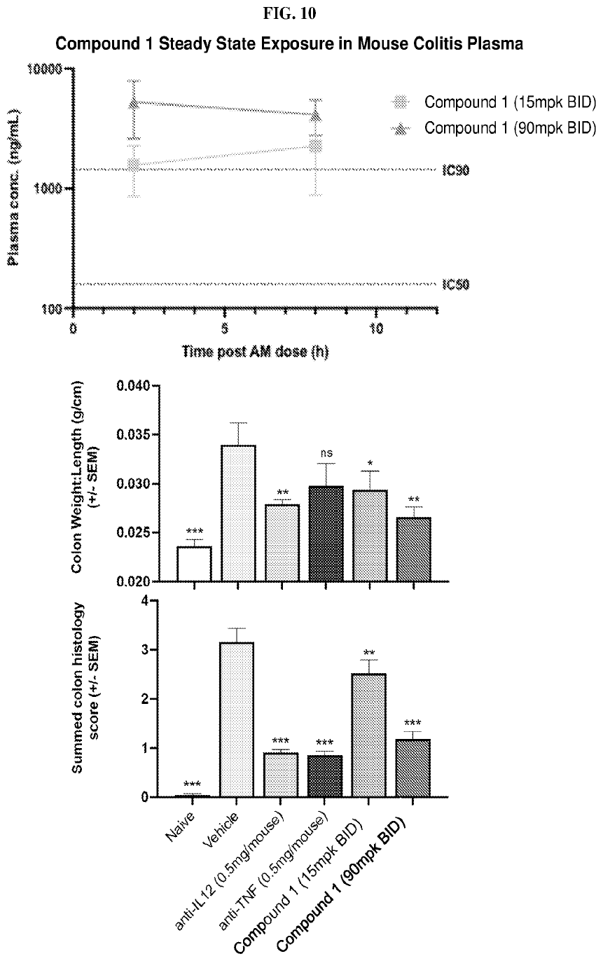

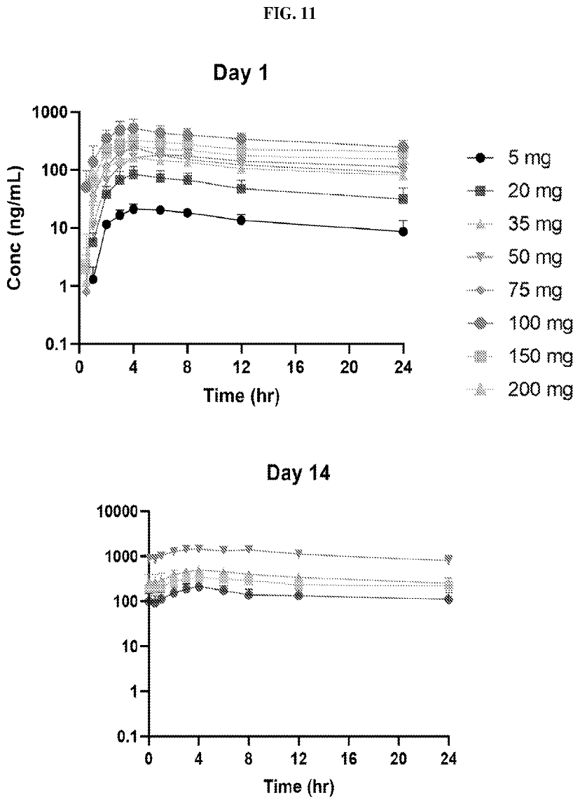

FIG. 10 shows the steady state exposure (plasma concentration) of Compound 1 administered to mice administered anti-CD40 (top panel) and the colon weights and colon histologies of the mice (bottom panel). We examined the ability of Compound 1 to achieve suppression of disease activity in a mouse anti-CD40-induced colitis model. It was found that doses of 15 mg/kg BID of Compound 1 provided ˜24 h of plasma levels in an amount sufficient to cover the TYK2 TC₅₀; doses of 90 mg/kg BID of Compound 1 provided ˜24 h of plasma levels sufficient to cover the TYK2 IC₉₀. Anti-CD40 was injected on day 0. Drug exposure was assessed on day 7 and a final necroscopy, colon assessment, and histology were performed. Summed colon histology included measures of inflammation, gland loss, erosion, and hyperplasia.

FIG. 11 shows pharmacokinetics of Compound 1 measured in healthy volunteers (PO; QD) Study 101 and Study 104. Peak mean plasma concentrations at median Tₘₐₓ were 4-6 hrs on day 1 and day 14. Cₘₐₓ and AUC₀₋₂₄ increased in an approximately dose-proportional manner from 5-75 mg. Oral T_{1/2} was consistent across doses: 16.5-30.7 hrs. The accumulation ratios for Cₘₐₓ and AUC were 2.2-2.9× and 2.5-3.2×, respectively.

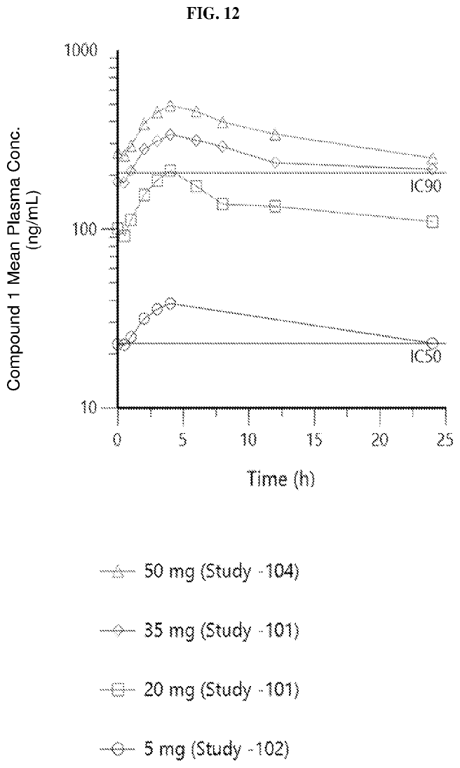

FIG. 12 shows steady-state plasma concentrations of Compound 1 based on steady-state exposures obtained from the 20 mg and 35 mg cohorts of Study 101 (Example 1), 5 mg cohort of Study 102 (Example 2) and the 50 mg cohort of Study 104. IC_{50/90} reference lines represent inhibitory concentrations in a human whole-blood TYK2-dependent IFNα-induced IP10 assay. The 35 and 50 mg doses cover the IC₉₀ for 24 h.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method of treating psoriasis or psoriatic arthritis in a patient in need thereof, comprising administering to a patient a therapeutically effective amount of Compound 1: 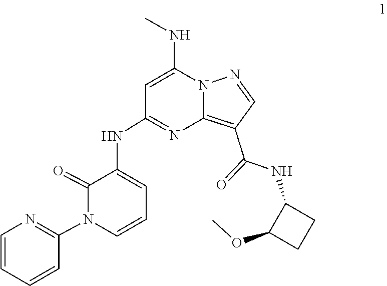 [SMILES: CNc1cc(Nc2cccn(-c3ccccn3)c2=O)nc2c(C(=O)N[C@@H]3CC[C@H]3OC)cnn12]
or a pharmaceutically acceptable salt thereof or a pharmaceutical composition thereof.

2. The method of claim 1, wherein the administration is daily for a period between about 1 day to about 7 days, about 1 week to about 3 weeks, about 3 weeks to about 6 weeks, about 6 weeks to about 9 weeks, about 9 weeks to 12 weeks, about 12 weeks to about 15 weeks, or about 15 weeks to about 18 weeks.

3. The method of claim 1 or 2, wherein a mean decrease in psoriasis area severity index (PASI) of about 50% to 75% is achieved.

4. The method of claim 1 or 2, wherein a mean decrease in psoriasis area severity index (PASI) of about 75% to 100% is achieved.

5. The method of any one of claims 1-4, wherein a Physician Global Assessment (PGA) of 0 or 1 is achieved.

6. The method of any one of claims 1-4, wherein a Physician Global Assessment (PGA) of 0 is achieved.

7. The method of any one of claims 1-6, wherein a Dermatology Life Quality Index (DLQI) score is reduced by an amount between about 1 to about 3, about 3 to about 6, about 6 to about 9, about 9 to about 12, about 12 to about 15, about 15 to about 18, about 18 to about 21, about 21 to about 24, about 24 to about 27, or about 27 to about 30.

8. The method of any one of claims 1-7, wherein a Dermatology Life Quality Index (DLQI) score is reduced by an amount greater than about 5.

9. The method of any one of claims 1-7, wherein a Dermatology Life Quality Index (DLQI) score is reduced by an amount between about 6 to about 9.

10. The method of any one of claims 1-9, wherein the patient's Body Surface Area (BSA) is reduced by a number of percentage points between about 1 to about 10, about 10 to about 20, about 20 to about 30, about 30 to about 40, about 40 to about 50, about 50 to about 60, about 60 to about 70, about 70 to about 80, about 80 to about 90, or about 90 to about 100.

11. The method of any one of claims 1-10, wherein a reduction in pain numeric rating scale (NRS) of about 1, 2, 3, 4, 5, 6, 7, 8, 9, or 10 is achieved.

12. The method of any one of claims 1-11, wherein a reduction in pruritus numeric rating scale (NRS) of about 1, 2, 3, 4, 5, 6, 7, 8, 9, or 10 is achieved.

13. The method of any one of claims 1-12, wherein an inhibition of IL-12/18-induced IFNγ production between about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, is achieved.

14. The method of any one of claims 1-13, wherein the patient has plaque psoriasis.

15. The method of claim 14, wherein the plaque psoriasis is moderate-to-severe-plaque psoriasis.

16. The method of any one of claims 1-15, wherein the patient has both psoriasis and psoriatic arthritis.

17. The method of any one of claims 1-15, wherein the patient has psoriatic arthritis.

18. The method of claim 17, wherein the method achieves at least an American College of Rheumatology (ACR) 20 response.

19. The method of claim 17, wherein the method achieves an improvement in tender joint count, swollen joint count, Patient Global Assessment of Psoriatic Arthritis, Patient Global Assessment of Psoriatic Arthritis Pain, Physician Global Assessment of Psoriatic Arthritis, Health Assessment Questionnaire-Disability Index (HAQ-DI) score, dactylitis count, Leed's Enthesitis Index, Minimal Disease Activity, Disease Activity Index for Psoriatic Arthritis (DAPSA), or a Psoriasis Area Severity Index (PASI)-75 at Week 12 among subjects with >3% body surface area (BSA) psoriatic involvement at Day 1, or a static Physician Global Assessment of 0 or 1 and at least a 2-point improvement from baseline at Week 12.

20. The method of claim 17, wherein the method achieves an improvement in circulating cytokines and/or an inflammatory biomarker, a 36-Item Short Form Health Survey, Functional Assessment of Chronic Illness—Fatigue, Disease Activity Score 28 with high-sensitivity C-Reactive Protein (hsCRP), Psoriatic Disease Activity Score at week 12 of treatment, Psoriatic Arthritis Response Criteria at week 12 of treatment, a mean decrease in psoriasis area severity index (PASI) of 25% or greater, or an improvement in BSA with psoriatic involvement at week 12, or an improvement in Physician Global Assessment of Psoriasis at week 12 of treatment.

21. A method of inhibiting of interferon gamma (IFNγ) production in a patient, comprising administering to a patient a therapeutically effective amount of Compound 1: 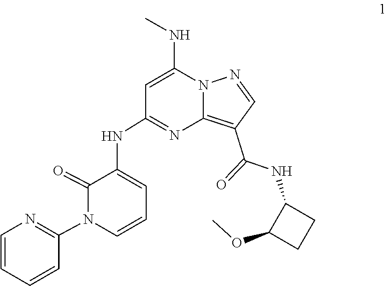 [SMILES: CNc1cc(Nc2cccn(-c3ccccn3)c2=O)nc2c(C(=O)N[C@@H]3CC[C@H]3OC)cnn12]
or a pharmaceutically acceptable salt thereof or a pharmaceutical composition thereof.

22. The method of claim 21, wherein the patient has psoriasis.

23. The method of claim 21, wherein the patient has psoriatic arthritis.

24. The method of claim 22, wherein the psoriasis is moderate to severe.

25. The method of claim 23, wherein the psoriatic arthritis is moderate to severe.

26. The method of any one of claims 1-25, wherein a Tₘₐₓ of Compound 1 in plasma is achieved in about 3 hours to about 6 hours.

27. The method of any one of claims 1-26, wherein a t_{1/2} of Compound 1 in plasma is achieved in about 17 hours to about 37 hours.

28. The method of any one of claims 1-27, wherein Compound 1, or a pharmaceutically acceptable salt thereof, is administered at a dose of up to about 200 mg to the patient.

29. The method of any one of claims 1-27, wherein Compound 1, or a pharmaceutically acceptable salt thereof, is administered at a dose of from about 20 mg to about 100 mg to the patient.

30. The method of any one of claims 1-27, wherein Compound 1, or a pharmaceutically acceptable salt thereof, is administered at a dose of about 2 mg, about 5 mg, about 10 mg, about 15 mg, or about 30 mg to the patient.

31. The method of any one of claims 1-30, wherein Compound 1, or a pharmaceutically acceptable salt thereof, is administered to the patient as a single daily dose.

32. The method of any one of claims 1-30, wherein Compound 1, or a pharmaceutically acceptable salt thereof, is administered to the patient in multiple daily doses.

33. The method of any one of claims 1-32, wherein Compound 1, or a pharmaceutically acceptable salt thereof, is administered to the patient daily for 2 weeks.

34. The method of any one of claims 1-32, wherein Compound 1, or a pharmaceutically acceptable salt thereof, is administered to the patient daily for 4 weeks.

35. The method of any one of claims 1-34, wherein Compound 1 is administered orally to the patient.

36. The method of any one of claims 1-35, wherein the patient is a human.

37. The method of any one of claims 1-36, wherein the patient has a PASI of at least about 12 prior to the administration.

38. The method of any one of claims 1-37, wherein the patient has a PGA of at least about 3 prior to the administration.

39. The method of any one of claims 1-38, wherein the patient has a BSA of at least about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, prior to the administration.

40. The method of any one of claims 1-39, wherein the psoriasis does not comprise erythrodermic, pustular, predominantly guttate psoriasis, or drug-induced psoriasis.

41. The method of any one of claims 1-40, wherein a serum Cₘₐₓ between about 25 ng/ml to about 50 ng/ml, about 50 ng/ml to about 100 ng/ml, about 100 ng/ml to about 125 ng/ml, about 125 ng/ml to about 150 ng/ml, about 150 ng/ml to about 175 ng/ml, about 175 ng/ml to about 200 ng/ml, about 200 ng/ml to about 225 ng/ml, about 225 ng/ml to about 250 ng/ml, about 250 ng/ml to about 275 ng/ml, or about 275 ng/ml to about 300 ng/ml, is achieved.

42. A method of treating moderate-to-severe plaque psoriasis in a patient in need thereof, comprising administering a therapeutically effective amount of Compound 1: 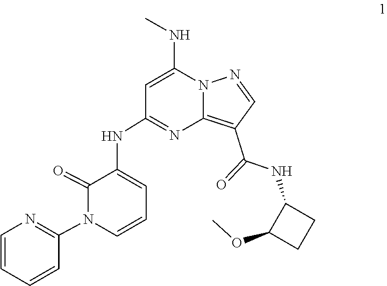 [SMILES: CNc1cc(Nc2cccn(-c3ccccn3)c2=O)nc2c(C(=O)N[C@@H]3CC[C@H]3OC)cnn12]
at a daily dose of from about 5 mg to about 30 mg.

43. The use of Compound 1: 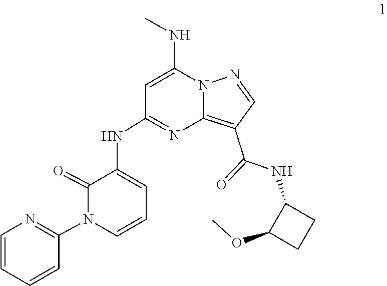 [SMILES: CNc1cc(Nc2cccn(-c3ccccn3)c2=O)nc2c(C(=O)N[C@@H]3CC[C@H]3OC)cnn12]
or a pharmaceutically acceptably salt thereof in the treatment of psoriasis or psoriatic arthritis.
